(19) United States
(12) Patent Application Publication
(10) Pub. No.: US 2005/0043545 A1
(43) Pub. Date: Feb. 24, 2005

(54) 4-(5-Membered)-heteroaryl acyl pyrrolidine derivatives as hcv inhibitors

(76) Inventors: Gianpalol Bravi, Research Triangle Park, NC (US); Helene Susanne Goodland, Stevenage (GB); David Haigh, Stevenage (GB); Charles David Hartley, Stevenage (GB); Victoria Lucy Helen Lovegrove, Stevenage (GB); Pritom Shah, Stevenage (GB); Martin John Slater, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/494,114

(22) PCT Filed: Oct. 30, 2002
(86) PCT No.: PCT/EP02/12172

(30) Foreign Application Priority Data
Nov. 2, 2001 (GB) 0126431.6
Nov. 2, 2001 (GB) 0126441.5
Aug. 19, 2002 (GB) 0219320.9
Aug. 19, 2002 (GB) 0219319.1

(51) Int. Cl. C07D 43/02

(52) U.S. Cl. 548/517; 548/530

(57) ABSTRACT

Novel anti-viral agents of Formula 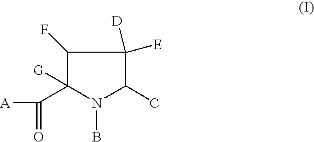 wherein:
A represents OR¹, NR¹R², or R¹ wherein R¹ and R² are hydrogen, C₁₋₆alkyl, aryl, heteroaryl, arylalkyl, or heteroarylalkyl; or R₁ and R² together with the nitrogen atom to which they are attached form a 5 or 6 membered saturated cyclic group;
B represents C(O)R³ wherein R₃ is C₁₋₆alkyl, aryl, heteroaryl, arylalkyl, or heteroarylalkyl;
C represents C₁₋₆alkyl, aryl, heteroaryl or heterocyclyl; D represents a saturated or unsaturated optionally substituted 5-membered heterocyclic ring;
E represents hydrogen or C₁₋₆alkyl;
F represents hydrogen, C₁₋₆alkyl, aryl or heteroaryl; and
G represents hydrogen, C₁₋₆alkyl, heterocyclylalkyl, arylalkyl or heteroarylalkyl; and salts and solvates thereof, processes for their preparation and methods of using them in HCV treatment are provided.

### FIELD OF THE INVENTION

The present invention relates to novel acyl pyrrolidine derivatives useful as anti-viral agents. Specifically, the present invention involves novel HCV inhibitors.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA—A Publication of the RNA Society. 1(5): 526-537, July, 1995 ). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p931-960; Raven Press, N.Y.). Following the termination codon at the end of the long ORF, there is a 3′ NTR which roughly consists of three regions: an ˜40 base region which is poorly conserved among various genotypes, a variable length poly(U)/polypyrimidine tract and a highly conserved 98 base element also called the “3′ X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al., (2000) Journal of Virology, 74(4), p.2046-2051). Thus, inhibition of NS5B RdRp activity (inhibition of RNA replication) is predicted to cure HCV infection.

Based on the foregoing, there exists a significant need to identify synthetic or biological compounds for their ability to inhibit HCV.

### SUMMARY OF THE INVENTION

The present invention involves compounds represented hereinbelow, pharmaceutical compositions comprising such compounds and use of the compounds in treating viral infection, especially HCV infection.

### DETAILED DESCRIPTION OF THE INVENTION

The present invention provides compounds of Formula (I): 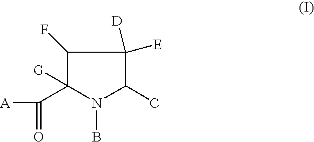
wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or R¹ and R² together with the nitrogen atom to which they are attached form a 5 or 6 membered saturated cyclic group;
B represents C(O)R³ wherein R³ is selected from the group consisting of C₁₋₆alkyl, aryl, heteroaryl, arylalkyl, and heteroarylalkyl;
C represents C₁₋₆alkyl, aryl, heteroaryl or heterocyclyl;
D represents a saturated or unsaturated 5-membered heterocyclic ring comprising one or more carbon atoms, each of which may independently be optionally substituted by R⁴ and R⁵, and one to four heteroatoms independently selected from N, optionally substituted by hydrogen, C₁₋₆alkyl, C(O)R³, SO₂R³, aryl, heteroaryl, arylalkyl, or heteroarylalkyl; O; and S, optionally substituted by one or two oxygen atoms; wherein the 5 membered ring may be attached at any endocyclic carbon atom, and may be optionally fused via two adjacent carbon atoms to a saturated or unsaturated 6 membered carbocyclic or heterocyclic ring which may itself be optionally substituted on a non-fused carbon atom by C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl;
R⁴ and R⁵ are independently selected from hydrogen, C₁₋₆alkyl, halo, OR⁸, C(O)NR⁶R⁷, C(O)R³, CO₂H, CO₂R³, NR⁶R⁷, NHC(O)R³, NRCO₂R³, NHC(O)NR¹R², SO₂NR¹R², SO₂R³, nitro, cyano, oxo, aryl, heteroaryl and heterocyclyl;
R⁶ and R⁷ are independently selected from hydrogen, C₁₋₆alkyl, aryl and heteroaryl; and
R⁸ represents hydrogen, C₁₋₆alkyl, arylalkyl, or heteroarylalkyl;
E represents hydrogen or C₁₋₆alkyl;
F represents hydrogen, C₁₋₆alkyl, aryl or heteroaryl; and
G represents hydrogen, C₁₋₆alkyl, heterocyclylalkyl, arylalkyl or heteroarylalkyl; and salts, solvates and esters thereof, provided that when A is OR¹ then R¹ is other than tert-butyl.

As used herein, “alkyl” refers to an optionally substituted hydrocarbon group. The alkyl hydrocarbon group may be linear, branched or cyclic, saturated or unsaturated. Where the alkyl hydrocarbon group is cyclic, it will be understood that there will be a minimum of 3 carbon atoms in the group. Preferably, the group is saturated. Preferred alkyl moieties are C₁₋₄alkyl. Optional substituents include C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, and heterocyclyl.

As used herein, “aryl” refers to an optionally substituted aromatic group with at least one ring having a conjugated pi-electron system, containing up to two conjugated or fused ring systems. “Aryl” includes carbocyclic aryl and biaryl groups, all of which may be optionally substituted. Preferred “aryl” moieties are unsubstituted, monosubstituted, disubstituted or trisubstituted phenyl. Preferred “aryl” substituents are selected from the group consisting of C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, heterocyclyl, CF₃, pyridine, phenyl, and NO₂.

As used herein, “heteroaryl” refers to an optionally substituted, 5 or 6 membered, aromatic group comprising one to four heteroatoms selected from N, O and S, with at least one ring having a conjugated pi-electron system, containing up to two conjugated or fused ring systems. Preferred “heteroaryl” moieties are unsubstituted, monosubstituted, disubstituted or trisubstituted thienyl, thiazolyl, pyridinyl and benzothiazolyl. Preferred “heteroaryl” substituents are selected from the group consisting of C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, heterocyclyl, CF₃, pyridine, phenyl, and NO₂.

As used herein, “heterocyclic” and “heterocyclyl” refer to an optionally substituted, 5 or 6 membered, saturated cyclic hydrocarbon group containing one to four, preferably one or two, heteroatoms selected from N, optionally substituted by hydrogen, C₁₋₆alkyl, C(O)R³, SO₂R³, aryl or heteroaryl; O; and S, optionally substituted by one or two oxygen atoms.

It will be appreciated that the compounds of the present invention may contain one or more asymmetric carbon atoms and may exist in racemic, diastereoisomeric, and optically active forms. All of these racemic compounds, enantiomers and diastereoisomers are contemplated to be within the scope of the present invention.

Preferably, A is OR¹ where R¹ is hydrogen; or A is NH₂; more preferably, A is OR¹ where R¹ is hydrogen.

Preferably, when B represents C(O)R³, R³ is aryl or heteroaryl; more preferably, R³ is optionally substituted phenyl; especially preferred is R³ represents phenyl substituted in the para-position by tert-butyl; most preferred is R³ represents phenyl substituted in the para-position by tert-butyl and optionally further substituted, preferably meta-substituted, by methyl, ethyl, methoxy, ethoxy, or halo, more preferably methoxy.

Preferably, C is selected from the group consisting of C₁₋₆alkyl, aryl and heteroaryl; more preferably, C is thien-2-yl, 1,3-thiazol-2-yl, 1,3-thiazol-4-yl, benzothiazol-2-yl, pyridin-2-yl or pyridin-3-yl.

Preferably, D is selected from the group (i) consisting of 1H-pyrrol-2-yl, 1H-pyrrol-3-yl, furan-2-yl, furan-3-yl, thien-2-yl, thien-3-yl; 1H-imidazol-2-yl, 1H-pyrazol-3-yl, 1H-pyrazol-5-yl, isoxazol-3-yl, isoxazol-5-yl, oxazol-2-yl, isothiazol-3-yl, isothiazol-5-yl, thiazol-2-yl, 1,3-dioxol-2-yl, 1,3-oxathiazol-2-yl, and 1,3-dithiol-2-yl, and partially or fully saturated derivatives thereof; each of which, where applicable, may be optionally substituted on a carbon atom by R⁴ and R⁵, on a nitrogen atom by hydrogen, C₁₋₆alkyl, C(O)R³, SO₂R³, aryl, heteroaryl, arylalkyl, or heteroarylalkyl, and on a sulphur atom by one or two oxygen atoms; and each of which may be optionally fused via two adjacent ring carbon atoms to a saturated or unsaturated 6 membered carbocyclic or heterocyclic ring which may itself be optionally substituted on a non-fused carbon atom by C₁₋₆alkyl, halo, OR⁸, C(O)NR⁶R₇, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl; more preferably by C₁₋₆alkyl, halo, OR⁸, C(O)NR⁶R⁷, CO₂R³, NR⁶R⁷, NHC(O)R³, NHCO₂R³, NHC(O)NR¹R², SO₂NR¹R², SO₂R³, nitro, oxo, aryl, heteroaryl and heterocyclyl;

or the group (ii) consisting of 1H-imidazol-4-yl, 1H-imidazol-5-yl, 1H-pyrazol-4-yl, isoxazol-4-yl, oxazol-4-yl, oxazol-5-yl, isothiazol-4-yl, thiazol-4-yl, thiazol-5-yl, 1,3-dioxol-4-yl, 1,3-oxathiazol-4-yl, 1,3-oxathiazol-5-yl, 1,3-dithiol-4-yl, 1H-1,2,3-triazol-4-yl, 1H-1,2,3-triazol-5-yl, 2H-1,2,3-triazol-4-yl, 1H-1,2,4-triazol-3-yl, 1H-1,2,4-triazol-5-yl, 4H-1,2,4-triazol-3-yl, 1,2,4-oxadiazol-5-yl, 1,2,4-oxadiazol-3-yl, 1,2,5-oxadiazol-3-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl, 1,3,4-thiadiazol-2-yl, 1,3,2-oxathiazol-4-yl, 1,3,2-oxathiazol-5-yl, 1,3,4-oxathiazol-2-yl, 1,3,4-oxathiazol-5-yl, 1H-tetrazol-5-yl, and 2H-tetrazol-5-yl, and partially or fully saturated derivatives thereof; each of which, where applicable, may be optionally substituted on a carbon atom by R⁴ and/or R⁵, on a nitrogen atom by hydrogen, C₁₋₆alkyl, C(O)R³, SO₂R³, aryl, heteroaryl, arylalkyl, or heteroarylalkyl, and on a sulphur atom by one or two oxygen atoms.

Preferably, when D is selected from group (i) and has a fused ring, the fused ring is selected from benzene, pyridine, pyrimidine, pyridazine and pyrazine.

In another preferred aspect, D is selected from the group consisting of 5-methyl-1,2,4-thiadiazol-3-yl; 1,2,4-thiadiazol-5-yl; 3-bromo-1,2,4-thiadiazol-5-yl; 3-methyl-1,2,4-oxadiazol-5-yl; 5-methyl-1,2,4-oxadiazol-3-yl; 5-methyl-1,3,4-oxadiazol-2-yl; 5-ethyl-1,2,4-oxadiazol-3-yl; 5-cyclopropyl-1,2,4-oxadiazol-3-yl; 3-methyl-isoxazol-5-yl; 1H-1,2,4-triazol-3-yl; 5-methyl-1H-1,2,4-triazol-3-yl; 1,2,4-oxadiazol-5-yl; 3-(4-fluorophenyl)-1,2,4-oxadiazol-5-yl; 1,2,4-oxadiazol-3-yl; 5(4H)-1,2,4-oxadiazolon-3-yl; 1,3,4-oxadiazol-2-yl; 1,3,4-thiadiazol-2-yl; isoxazol-5-yl; 3-methyl-isoxazol-5-yl; 3-methyl-pyrazol-5-yl; thiazol-2-yl; 1-methyl-1H-tetrazol-5-yl; benzothiazol-2-yl; and benzoxazol-2-yl.

In an alternative preferred aspect, 1H-1,2,4-triazol-3-yl; 5-methyl-1H-1,2,4-triazol-3-yl; 1,2,4-oxadiazol-5-yl; 3-methyl-1,2,4-oxadiazol-5-yl; 3-(4-fluorophenyl)-1,2,4-oxadiazol-5-yl; 1,2,4-oxadiazol-3-yl; 5-methyl-1,2,4-oxadiazol-3-yl; 5(4H)-1,2,4-oxadiazolon-3-yl; 1,3,4-oxadiazol-2-yl; 1,3,4-thiadiazol-2-yl; isoxazol-5-yl; 3-methyl-isoxazol-5-yl; 3-methyl-pyrazol-5-yl; thiazol-2-yl; 1-methyl-1H-tetrazol-5-yl; benzothiazol-2-yl; and benzoxazol-2-yl; more preferably D is selected from the group consisting of 1H-1,2,4-triazol-3-yl, 5-methyl-1H-1,2,4-triazol-3-yl, 1,2,4-oxadiazol-5-yl, and 3-methyl-1,2,4-oxadiazol-5-yl.

Preferably, E is hydrogen.

Preferably, F is hydrogen.

Preferably, G is selected from the group consisting of C₁₋₆alkyl, arylalkyl and heteroarylalkyl; more preferably, G represents isobutyl, benzyl or methyl; most preferably G represents isobutyl or benzyl.

It is to be understood that the present invention covers all combinations of suitable, convenient and preferred groups described herein.

In one preferred aspect, the present invention provide compounds of Formula (I) represented by Formula (Ia) 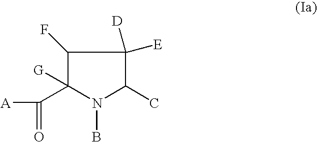
wherein:
A represents OR₁, NR₁R₂, or R₁ wherein R₁ and R₂ are independently selected from the group consisting of hydrogen, C₁₋₆alkyl, aryl, heteroaryl, arylalkyl, and heteroarylalkyl; or R₁ and R₂ together with the nitrogen atom to which they are attached form a 5 or 6 membered saturated cyclic group;
B represents C(O)R₃ wherein R₃ is selected from the group consisting of C₁₋₆alkyl, aryl, heteroaryl, arylalkyl, and heteroarylalkyl;
C represents C₁₋₆alkyl, aryl, heteroaryl or heterocyclyl;
D represents a saturated or unsaturated 5-membered heterocyclic ring comprising one or more carbon atoms, each of which may independently be optionally substituted by R₄ and R₅, and one to four heteroatoms independently selected from N, optionally substituted by hydrogen, C₁₋₆alkyl, C(O)R₃, SO₂R₃, aryl, heteroaryl, arylalkyl, or heteroarylalkyl; O; and S, optionally substituted by one or two oxygen atoms; wherein the 5 membered ring may be attached at any endocyclic carbon atom, and may be optionally fused to a saturated or unsaturated 6 membered carbocyclic or heterocyclic ring which may itself be optionally substituted on a non-fused carbon atom by C₁₋₆alkyl, halo, OR₈, C(O)NR₆R₇, CO₂R₃, NR₆R₇, NHC(O)R₃, NHCO₂R₃, NHC(O)NR₁R₂, SO₂NR₁R₂, SO₂R₃, nitro, oxo, aryl, heteroaryl and heterocyclyl;

R₄ and R₅ are independently selected from hydrogen, C₁₋₆alkyl, halo, OR₈, C(O)NR₆R₇, CO₂R₃, NR₆R₇, NHC(O)R₃, NHCO₂R₃, NHC(O)NR₁R₂, SO₂NR₁R₂, SO₂R₃, nitro, oxo, aryl, heteroaryl and heterocyclyl;
R₆ and R₇ are independently selected from hydrogen, C₁₋₆alkyl, aryl and heteroaryl; and
R₈ represents hydrogen, C₁₋₆alkyl, arylalkyl, or heteroarylalkyl;
E represents hydrogen or C₁₋₆alkyl;
F represents hydrogen, C₁₋₆alkyl, aryl or heteroaryl; and
G represents hydrogen, C₁₋₆alkyl, heterocyclylalkyl, arylalkyl or heteroarylalkyl; and salts and solvates thereof, provided that when A is OR₁ then R₁ is other than tert-butyl.

In a further preferred aspect, the present invention provides compounds of Formula (I) represented by Formula (Ib) 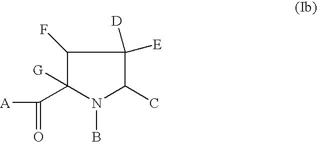
wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or R¹ and R² together with the nitrogen atom to which they are attached form a 5 or 6 membered saturated cyclic group;
B represents C(O)R³ wherein R³ is selected from the group consisting of C₁₋₆alkyl, aryl, heteroaryl, arylalkyl, and heteroarylalkyl;
C represents C₁₋₆alkyl, aryl, heteroaryl or heterocyclyl;
D represents 1H-1,2,4-triazol-3-yl; 1,2,4-oxadiazol-5-yl; 1,2,4-oxadiazol-3-yl; 1,3,4-oxadiazol-2-yl; 1,3,4-thiadiazol-2-yl; isoxazol-5-yl; pyrazol-5-yl; thiazol-2-yl, 1H-tetrazol-5-yl; benzothiazol-2-yl; or benzoxazol-2-yl each of which may independently be optionally substituted on a carbon atom by R⁴, and, where applicable, may independently be optionally substituted on an N atom by hydrogen, C₁₋₆alkyl, C(O)R³, SO₂R³, aryl, heteroaryl, arylalkyl, or heteroarylalkyl; R⁴ is selected from hydrogen, C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, oxo, aryl, heteroaryl and heterocyclyl;
R⁶ and R⁷ are independently selected from hydrogen, C₁₋₆alkyl, aryl and heteroaryl; and
R⁸ represents hydrogen, C₁₋₆alkyl, arylalkyl, or heteroarylalkyl;
E represents hydrogen or C₁₋₆alkyl;
F represents a hydrogen, C₁₋₆alkyl, aryl or heteroaryl; and
G represents hydrogen, C₁₋₆alkyl, heterocyclylalkyl, arylalkyl or heteroarylalkyl; and salts, solvates and esters thereof, provided that when A is OR¹ then R¹ is other than tert-butyl.

In another preferred aspect, the present invention provides compounds of Formula (I) represented by Formula (Ic) 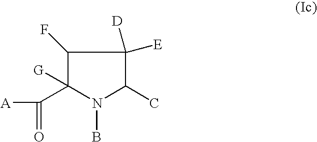
wherein:
A represents OR₁, NR₁R₂, or R₁ wherein R₁ and R₂ are independently selected from the group consisting of hydrogen, C₁₋₆alkyl, aryl, heteroaryl, arylalkyl, and heteroarylalkyl; or R₁ and R₂ together with the nitrogen atom to which they are attached form a 5 or 6 membered saturated cyclic group;
B represents C(O)R₃ wherein R₃ is selected from the group consisting of C₁₋₆alkyl, aryl, heteroaryl, arylalkyl, and heteroarylalkyl;
C represents C₁₋₆alkyl, aryl, heteroaryl or heterocyclyl;
D represents 1H-1,2,4-triazol-3-yl or 1,2,4-oxadiazol-5-yl, each of which may independently be optionally substituted on a carbon atom by R₄, and, where applicable, may independently be optionally substituted on an N atom by hydrogen, C₁₋₆alkyl, C(O)R₃, SO₂R₃, aryl, heteroaryl, arylalkyl, or heteroarylalkyl; R₄ is selected from hydrogen, C₁₋₆alkyl, halo, OR₈, C(O)NR₆R₇, CO₂R₃, NR₆R₇, NHC(O)R₃, NHCO₂R₃, NHC(O)NR₁R₂, SO₂NR₁R₂, SO₂R₃, nitro, oxo, aryl, heteroaryl and heterocyclyl;
R₆ and R₇ are independently selected from hydrogen, C₁₋₆alkyl, aryl and heteroaryl; and
R₈ represents hydrogen, C₁₋₆alkyl, arylalkyl, or heteroarylalkyl;
E represents hydrogen or C₁₋₆alkyl;
F represents a hydrogen, C₁₋₆alkyl, aryl or heteroaryl; and
G represents hydrogen, C₁₋₆alkyl, heterocyclylalkyl, arylalkyl or heteroarylalkyl; and salts and solvates thereof, provided that when A is OR₁ then R₁ is other than tert-butyl.

Preferred compounds useful in the present invention are selected from the group consisting of:
rel-(2S,4R,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid;
rel-(2S,4S,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid;
rel-(2S,4S,5R)-1-(4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methyl-1,2,4-oxadiazol-5-yl)-5-(1,3-thiazol-2-yl)pyrrolydine-2-carboxylic acid;
rel-(2S,4S,5R)-1-(4-tert-butylbenzoyl)-2-isobutyl-5-(1,3-thiazol-2-yl)-4-(1H-1,2,4-triazol-3-yl)pyrrolidine-2-carboxylic acid;
rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-(1,2,4-oxadiazol-5-yl)-5-(1,3-thiazol-2-yl)pyrrolidine-2-carboxylic acid; and
rel-(2S,4R,5R)-1-(4-tert-butylbenzoyl)-2-isobutyl-4-(5-methyl-1H-1,2,4-triazol-3-yl)-5-thien-2-yl-pyrrolidine-2-carboxylic acid;
rel-(2S,4R,5R)-1-(4-tert-butylbenzoyl)-2-isobutyl-4-(3-methyl-1,2,4-oxadiazol-5-yl)-5-thien-2-ylpyrrolidine-2-carboxylic acid;
rel-(2S,4S,5R)-1-(4-tert-butylbenzoyl)-2-isobutyl-4-(5-methyl-1,2,4-oxadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(1,2,4-oxadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxamide;
rel-(2S,4S,5R)-1-(3-bromo-4-tert-butylbenzoyl)-2-isobutyl-4-(1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-[5(4H)-1,2,4-oxadiazolon-3-yl)-5-(1,3-thiazol-2-yl)pyrrolidine-2-carboxylic acid;
rel-(2S,4R,5R)-1-(4-tert-butylbenzoyl)-2-isobutyl-4-(isoxazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methylisoxazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methylpyrazol-5-yl)-5-(1,3-thiazol-2-yl)pyrrolidine-2-carboxylic acid;
rel-(2S,4R,5R)-1-(4-tert-butylbenzoyl)-2-isobutyl-4-(1,3-thiazol-2-yl)-5-(1,3-thiazol-2-yl)pyrrolidine-2-carboxylic acid;
rel-(2S,4S,5R)-1-(4-tert-butylbenzoyl)-2-isobutyl-4-(1,3-thiazol-2-yl)-5-(1,3-thiazol-2-yl)pyrrolidine-2-carboxylic acid;
rel-(2S,4R,5R)-2-isobutyl-1-(4-tert-butylbenzoyl)-4-(1,3,4-oxadiazol-2-yl)-5-(1,3-thiazol-2-yl)-pyrrolidine-2-carboxylic acid;
rel-(2S,4S,5R)-2-isobutyl-1-(4-tert-butylbenzoyl)-4-(1,3,4-oxadiazol-2-yl)-5-(1,3-thiazol-2-yl)-pyrrolidine-2-carboxylic acid;
rel-(2S,4R,5R)-2-isobutyl-1-(4-tert-butylbenzoyl)-4-(1,3,4-thiadiazol-2-yl)-5-(1,3-thiazol-2-yl)-pyrrolidine-2-carboxylic acid;
rel-(2R,4S,5R)-2-benzyl-1-(4-tert-butylbenzoyl)-4-(1,2,4-oxadiazol-5-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(1-methyl-1H-tetrazol-5-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(benzothiazol-2-yl)-5-(1,3-thiazol-2-yl)-pyrrolidine-2-carboxylic acid;
rel-(2S,4R,5R)-2-isobutyl-1-(3-bromo-4-tert-butylbenzoyl)-4-(benzothiazol-2-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(benzoxazol-2-yl)-5-(1,3-thiazol-2-yl)-pyrrolidine-2-carboxylic acid;
rel-(2S,4R,5R)-2-isobutyl-1-(3-bromo-4-tert-butylbenzoyl)-4-(benzoxazol-2-yl)-5-(1,3-thiazol-2-yl)-pyrrolidine-2-carboxylic acid;
rel-(2S,4S,5R)-1-(4-tert-butylbenzoyl)-2-isobutyl-4-(1,2,4-oxadiazol-5-yl)-5-pyridin-2-yl-pyrrolidine-2-carboxylic acid;
rel-(2S,4R,5R)-1-(4-tert-butylbenzoyl)-2-isobutyl-4-(1,2,4-oxadiazol-5-yl)-5-pyridin-2-yl-pyrrolidine-2-carboxylic acid;
rel-(2S,4S,5R)-1-(4-tert-butylbenzoyl)-2-isobutyl-4-(1,2,4-oxadiazol-5-yl)-5-(1,3thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid;
rel-(2S,4S,5R)-1-(3-chloro-4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-1-(3-chloro-4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxamide;
rel-(2S,4R,5R)-2-isobutyl-1-(3-methoxy-4-tert-butylbenzoyl)-4-(5-methyl-1,2,4-oxadiazol-3-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-oxadiazol-3-yl)-5-(1,3-thiazol-2-yl)pyrrolidine-2-carboxylic acid;
rel-(2S,4S,5R)-5-(benzothiazol-2-yl)-2-isobutyl-1-(4-tert-butylbenzoyl)-4-(1,2,4-oxadiazol-5-yl)-pyrrolidine-2-carboxylic acid;
rel-(2S,4S,5R)-5-(benzothiazol-2-yl)-2-isobutyl-1-(3-bromo-4-tert-butyl-benzoyl)-4-(1,2,4-oxadiazol-5-yl)-pyrrolidine-2-carboxylic acid;
rel-(2S,4S,5R)-1-(3-bromo-4-tert-butylbenzoyl)-4-[3-(4-fluorophenyl)-1,2,4-oxadiazol-5yl]-2-isobutyl-5-(1,3-thiazol-2-yl)pyrrolidine-2-carboxylic acid;
rel-(2S,4S,5R)-1-(4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid;
rel-(2S,4R,5R)-1-(4-tert-butylbenzoyl)-4-[3-bromo-1,2,4-thiadiazol-5-yl]-2-isobutyl-5-(1,3-thiazol-2-yl)pyrrolidine-2-carboxylic acid;
rel-(2S,4S,5R)-1-(4-tert-butylbenzoyl)-4-(3-bromo-1,2,4-thiadiazol-5-yl)-2-isobutyl-5-(1,3-thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-thien-2-ylpyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-thien-2-ylpyrrolidine-2-carboxylic acid;
rel-(2S,4R,5R)-2-isobutyl-1-(3-methoxy-4-tert-butylbenzoyl)-4-methyl-4-(5-methyl-1,2,4-oxadiazol-3-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-oxadiazol-3-yl)-5-thien-2-ylpyrrolidine-2-carboxylic acid;
rel-(2S,4R,5R)-2-isobutyl-1-(3-methoxy-4-tert-butylbenzoyl)-4-(5-methyl-1,2,4-oxadiazol-3-yl)-5-thien-2-ylpyrrolidine-2-carboxylic acid;
rel-(2S,4S,5R)-2-isobutyl-1-(4-tert-butylbenzoyl)-4-(3-methyl-1,2,4-oxadiazol-5-yl)-5-pyridin-3-ylpyrrolidine-2-carboxylic acid, trifluoroacetate salt;
rel-(2S,4S,5R)-2-isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(5-methyl-1,2,4-oxadiazol-3-yl)-5-(1,3-thiazol-4-yl)pyrrolidine-2-carboxylic acid;
rel-(2S,4R,5R)-1-(4-tert-butylbenzoyl)-4-(1,2,4-thiadiazol-5-yl)-2-isobutyl-5-(1,3-thiazol-2-yl)pyrrolidine-2-carboxylic acid;
rel-(2S,4R,5R)-2-isobutyl-1-(3-methoxy-4-tert-butylbenzoyl)-4-(5-methyl-1,3,4-oxadiazol-2-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl -1,3,4-oxadiazol-2-yl)-5-(1,3-thiazol-2-yl)pyrrolidine-2-carboxylic acid;
rel-(2S,4S,5R)-2-isobutyl-1-(4-tert-butylbenzoyl)-4-(5-methyl-1,3,4-oxadiazol-2-yl)-5-(1,3-thiazol-2-yl)pyrrolidine-2-carboxylic acid;
rel-(2S,4R,5R)-2-isobutyl-1-(4-tert-butylbenzoyl)-4-(5-methyl-1,3,4-oxadiazol-2-yl)-5-(1,3-thiazol-2-yl)pyrrolidine-2-carboxylic acid;
rel-(2S,4R,5R)-4-(5-ethyl-1,2,4-oxadiazol-3-yl)-2-isobutyl-1-(3-methoxy4-tert-butylbenzoyl)-5-(1,3-thiazol-2-yl)pyrrolidine-2-carboxylic acid;
rel-(2S,4R,5R)-4-(5-cyclopropyl-1,2,4-oxadiazol-3-yl)-2-isobutyl-1-(3-methoxy-4-tert-butylbenzoyl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-thiadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(5-methyl-1,2,4-thiadiazol-3-yl)-5-(1,3-thiazol-2-yl)pyrrolidine-2-carboxylic acid;
rel-(2S,4S,5R)-1-(3-methoxy-4-tert-butylbenzoyl)-2-methyl-4-(3-methyl-1,2,4-oxadiazol-5-yl)-5-(1,3-thiazol-2-yl)pyrrolidine-2-carboxylic acid;
rel-(2S,4R,5R)-1-(1-bromo-4-tert-butylbenzoyl)-4-(3-methyl-1,2,4-oxadiazol-5-yl)-2-isobutyl-5-pyridin-2-ylpyrrolidine-2-carboxylic acid;
rel-(2S,4R,5R)-2-isobutyl-1-(3-methoxy-4-tert-butylbenzoyl)-4-(3-methyl-isoxazol-5-yl)-5-(1,3-thiazol-2-yl)pyrrolidine-2-carboxylic acid;
rel-(2S,4R,5R)-2-isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-(1,3-thiazol-4-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-4-yl)pyrrolidine-2-carboxylic acid;
   and salts, solvates and esters, and individual enantiomers thereof.

In a preferred aspect, the present invention provides compounds of Formula (I) selected from the group consisting of Examples 1 to 47 hereinafter defined, and salts, solvates and esters, and where appropriate, individual enantiomers thereof In a further preferred aspect, the present invention provides compounds of Formula (I) selected from the group consisting of Examples 1 to 11 hereinafter defined, and salts, solvates and esters, and where appropriate, individual enantiomers thereof.

Also included in the present invention are pharmaceutically acceptable salt complexes. The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

The present invention also relates to pharmaceutically acceptable esters of the compounds of Formula (I), (Ia), (Ib) and (Ic), for example carboxylic acid esters —COOR, in which R is selected from straight or branched chain alkyl, for example n-propyl, n-butyl, alkoxyalkyl (e.g. methoxymethyl), aralkyl (e.g. benzyl), aryloxyalkyl (e.g. phenoxymethyl), aryl (e.g. phenyl optionally substituted by halogen, C₁₋₄alkyl or C₁₋₄alkoxy or amino). Unless otherwise specified, any alkyl moiety present in such esters preferrably contains 1 to 18 cabon atoms, particularly 1 to 4 carbon atoms. Any aryl moiety present in such esters preferrably comprises a phenyl group.

It will further be appreciated that certain compounds of the present invention may exist in different tautomeric forms. All tautomers are contemplated to be within the scope of the present invention.

Compounds of Formula (I) may be prepared from a compound of Formula (II) 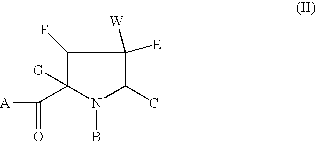
in which A, B, C, E, F and G are as defined above for Formula (I); W represents —CN, —CO₂H, —CO₂R⁹, —COR¹⁰, —C(O)NR⁶R⁷, or —C(O)Hal; and R⁹ represents C₁₋₆alkyl, or arylalkyl; and R¹⁰ represents C₁₋₆alkyl; by any suitable method for the conversion of the moiety W into the moiety D of formula (I). Suitable methods for the conversion of W into D may be found in the chemical literature, for example those described in Comprehensive Heterocyclic Chemistry, Edited by A. R. Katritzky and C. W. Rees, Pergamon 1984; WO 2001/28996 and WO 99/54299.

For example, the conversion of W, when W is —CONR⁶R⁷ and R⁶ and R⁷ are both hydrogen, into D, when D is a 1(H)-1,2,4-triazol-3-yl group, may be achieved by reacting the compound of Formula (II) with (1,1-dimethoxymethyl)dimethylamine followed by hydrazine in acetic acid.

The conversion of W into D may suitably include conversion of a particular W moiety into an intermediate moiety, W¹, by methods well known in the art, for example those described in Comprehensive Heterocyclic Chemistry, Edited by A. R. Katritzky and C. W. Rees, Pergamon 1984; WO 2001/28996 and WO 99/54299. For example, when W is —C(O)NR⁶R⁷ this group may be converted into W¹ is —C(S)NR⁶R⁷ by heating under reflux with Lawesson's reagents. When W is —CO₂R⁹, this group may be converted into W¹ is —CONHNH₂ by heating under reflux with hydrazine hydrate. When W is —CONHNH₂, this group may be converted into W¹ is CONHN═CHOEt by reaction with triethyl orthoformate. Thereafter, W¹ may be converted into D as described above for W.

Compounds of Formula (II) may be prepared by reaction of a compound of Formula (III) 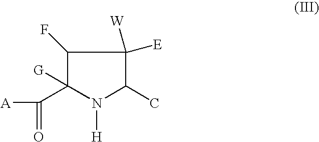
in which A, C, E, F and G are as defined above for Formula (I); and W is as defined above for Formula (II); with a suitable acylating agent, for example R³C(O)-hal, wherein hal is a halo atom, preferably chloro or bromo. Preferably the reaction is carried out in a suitable solvent, for example dichloromethane, in the presence of a suitable base, for example triethylamine.

Compounds of Formula (III) may be prepared by reaction of a compound of Formula (IV) 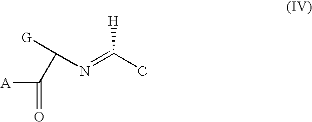
wherein A, C and G are as defined for Formula (I) above; with a compound of Formula (V) 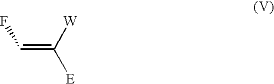
wherein E and F are as defined for Formula (I) and W is as defined for Formula (II) above. Preferably, the reaction is carried out in a suitable solvent, for example THF, in the presence of a Lewis acid catalyst, such as lithium bromide, and a base, such as triethylamine.

The C4-epimer of a compound of Formula (III) may be isolated from the resultant mixture of the reaction of a compound of Formula (IV) and Formula (V) described above, for example by purification by column chromatography using an appropriate eluant.

Compounds of Formula (I) may also be prepared by reaction of a compound of Formula (VI) 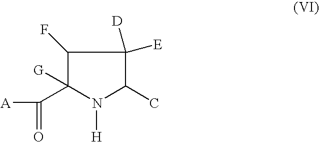
in which A, C, D, E, F and G are as defined above for Formula (I); with a suitable acylating agent, for example R³C(O)-hal, wherein hal is a halo atom, preferably chloro or bromo. Preferably the reaction is carried out in a suitable solvent, for example dichloromethane, in the presence of a suitable base, for example triethylamine.

Compounds of Formula (VI) may prepared by reaction of a compound of Formula (VII) 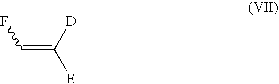
wherein E, D and F are as described for Formula (I) above; with a compound of Formula (IV). Preferably, the reaction is carried out in a suitable solvent, for example THF, in the presence of a Lewis acid catalyst, such as lithium bromide, and a base, such as triethylamine.

Compounds of Formula (VI) may also prepared from a compound of Formula (III) by any suitable method for the conversion of the moiety W into the moiety D of formula (I) as previously described.

Compounds of Formula (I) in which A is NR¹R² may be prepared from compounds of Formula (I) in which A is OH by treatment with a suitable amine source under standard amide bond forming conditions well established in the art. For example, a compound of Formula (I) in which A is NH₂ may be prepared from a compound of Formula (I) in which A is OH by reaction with ammonium chloride in the presence of a suitable base, such as diisopropylethylamine, together with a suitable dehydrating agent, such as HATU. The reaction may conveniently be carried out in any suitable solvent, for example N,N-dimethylformamide.

Compounds of Formula (I) may be converted into other compounds of Formula (I) by manipulation of the group D. For example, a compound of Formula (I) in which D represents 3-bromo-1,2,4-thiadiazol-5-yl may be converted into a compound of Formula (I) in which D represents 1,2,4-thiadiazol-5-yl by reaction with a suitable debrominating agent, for example ammonium formate, optionally in the presence of a catalyst, for example 10% palladium on carbon, in a suitable solvent, for example ethanol.

It will be appreciated that compounds of Formula (I), (II), (III) and/or (VI) which exist as diastereoisomers may optionally be separated by techniques well known in the art, for example by column chromatography.

It will also be appreciated that the present invention provides a method for the interconversion of the rel-(2S,4S,5R)-diastereoisomer of a compound of formula (I), (II), (III) and/or (VI) into the rel-(2S,4R,5R)-diastereoisomer. For example the conversion of the rel-(2S,4S,5R)-diastereoisomer of a compound of Formula (VI) when D is 3-methyl-1,2,4-oxadiazol-5-yl into the rel-(2S,4R,5R)-diastereoisomer is accomplished by treatment of the rel-(2S,4S,5R)-diastereoisomer with a suitable base, such as aqueous sodium hydroxide, in the presence of a suitable solvent, such as methanol. Such base-catalysed epimerisation may be used for the interconversion of the rel-(2S,4S,5R)-diastereoisomer of a compound of formula (I), (II), (III) and/or (VI) in which E represents hydrogen, into the rel-(2S,4R,5R)-diastereoisomer, where appropriate.

It will be appreciated that racemic compounds of Formula (I), (II), (III) and/or (VI) may be optionally resolved into their individual enantiomers. Such resolutions may conveniently be accomplished by standard methods known in the art. For example, a racemic compound of Formula (I), (II), (III) and/or (VI) may be resolved by chiral preparative HPLC. Alternatively, racemic compounds of Formula (I), (II), (III) and/or (VI) may be resolved by standard diastereoisomeric salt formation with a chiral acid or base reagent as appropriate. Such techniques are well established in the art For example, a racemic compound of Formula (III) where W is C(O)NR⁶R⁷ and R⁶ and R⁷ are both hydrogen may be resolved by treatment with a chiral acid such as (−)-di-O,O′-p-tolyl-L-tartaric acid.

Compounds of Formula (IV), (V) and (VII) are known in the art or may be prepared by standard literature procedures.

With appropriate manipulation and protection of any chemical functionality, synthesis of compounds of Formula (I) is accomplished by methods analogous to those above and to those described in the Experimental section. Suitable protecting groups can be found, but are not restricted to, those found in T W Greene and P G M Wuts ‘Protective Groups in Organic Synthesis’, 3^{rd} (1999), J Wiley and Sons.

### EXAMPLES

### Intermediate 1

### 2-[N-(1,3-Thiazol-2-ylmethylene)amino]-4-methylpentanoic acid, tert-butyl ester

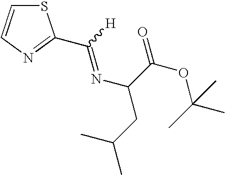

A stirred mixture of 2-amino-4-methyl-pentanoic acid tert-butyl ester, hydrochloride salt (5.00 g, 22.34 mmol), 1,3-thiazole-2-carboxaldehyde (2.53 g, 22.34 mmol) and triethylamine (3.10 mL, 22.3 mmol) in dichloromethane (60 mL) were heated under reflux under nitrogen for 19 hours. The reaction mixture was allowed to cool to room temperature, washed twice with water, dried over Na₂SO₄ and evaporated to give the title compound as an oil.

¹H NMR (CDCl₃): δ 8.46 (s, 1H), 7.94 (d, 1H), 7.44 (dd, 1H), 4.07 (dd, 1H), 1.89-1.74 (m, 2H), 1.64-1.52 (m, 1H), 1.48 (s, 9H), 0.96 (d, 3H) and 0.90 (d, 3H).

### Intermediate 2

### rel-(2S,4S,5R)-4-(Aminocarbonyl)-2-isobutyl-5-(1,3-thiazol-2-yl)pyrrolidine-2-carboxylic acid, tert-butyl ester

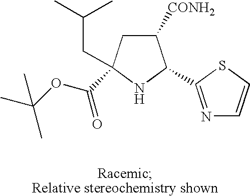

To a cooled (−5°), stirred solution of 2-[N-(1,3-thiazol-2-ylmethylene)amino]-4-methylpentanoic acid tert-butyl ester (Intermediate 1; 6.21 g, 21.99 mmol) in anhydrous THF (30 mL) under nitrogen, was added acrylamide (2.34 g, 32.98 mmol) followed by lithium bromide (3.82 g, 43.98 mmol). The resultant mixture was stirred at −5° C. for 5 minutes and then triethylamine (4.57 mL, 32.98 mmol) was added and stirring was continued at ambient temperature for 21 hours. Aqueous ammonium chloride was added with rapid stirring and the resulting mixture was extracted twice with ethyl acetate. The extracts were combined and washed twice with water and once with brine. A precipitate formed in the ethyl acetate solution and this was filtered off, washed with a little ethyl acetate and finally dried in vacuo to give the title compound as a solid. Additional quantities of the title compound were obtained from the ethyl acetate solution by further crystallisation and evaporation.

MS calcd for (C₁₇H₂₇N₃O₃S+H)⁺: 354. Found: (M+H)⁺=354

¹H NMR (DMSO-d₆): δ 7.64 (d, 1H), 7.54 (d, 1H), 7.24 (br.s, 1H), 6.68 (br.s, 1H), 4.68 (t, 1H), 3.43 (d, 1H), 3.23 (q, 1H), 2.41 (dd, 1H), 1.85 (dd, 1H), 1.68 (m, 1H), 1.62-1.52 (m, 2H), 1.43 (s, 9H), 0.90 (d, 3H) and 0.86 (d, 3H).

### Intermediate 3

### rel-(2S,4S,5R)-4-(Aminocarbonyl)-1-(4-tert-butylbenzoyl)-2-isobutyl-5-(1,3-thiazol-2-yl)-pyrrolidine-2-carboxylic acid, tert-butyl ester

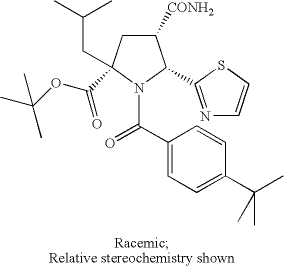

To a stirred solution rel-(2S,4S,5R)-4-(aminocarbonyl)-2-isobutyl-5-(1,3-thiazol-2-yl)pyrrolidine-2-carboxylic acid tert-butyl ester (Intermediate 2; 4.00 g, 11.32 mmol) in anhydrous dichloromethane (100 mL) was added triethylamine (1.97 mL, 14.15 mmol) and 4-tert-butyl-benzoyl chloride (2.70 g, 13.58 mmol). This mixture was stirred for 2.5 hours and was then washed with saturated aqueous sodium bicarbonate solution. The organic phase was dried (Na₂SO₄) and evaporated. The residue was purified by chromatography on silica gel using ethyl acetate-cyclohexane (3:1 v/v) as eluent to provide the title compound as foam.

MS calcd for (C₂₈H₃₉N₃O₄S+H)⁺: 514. Found (M+H)⁺=514.

¹H NMR (CDCl₃): δ 7.39 (d, 1H), 7.24 (½AA′BB′, 2H), 7.18 (d, 1H), 7.02 (½AA′BB′, 2H), 5.78 (d, 1H), 5.65 (v.br.s, 1H), 5.24 (v.br.s, 1H), 3.65 (m, 1H), 3.02 (t, 1H), 2.37 (dd, 1H), 2.28-2.12 (m, 2H), 1.95 (m, 1H), 1.51 (s, 9H), 1.27 (s, 9H) and 1.08 (d, 6H).

### Intermediate 4

### 2-[N-(Thien-2-ylmethylene)amino]-4-methylpentanoic acid, tert-butyl ester

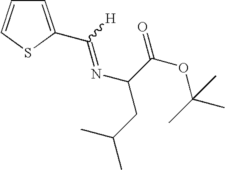

The title compound was prepared according to the method described for Intermediate 1, substituting thiophene-2-carboxaldehyde in place of thiazole-2-carboxaldehyde.

¹H NMR (CDCl₃): δ 8.38 (s, 1H), 7.43 (dt, 1H), 7.36 (dd, 1H), 7.08 (dd, 1H), 3.94 (dd, 1H), 1.87-1.71 (m, 2H), 1.59 (m, 1H), 1.46 (s, 9H), 0.95 (d, 3H) and 0.89 (d, 3H).

### Intermediate 5

### rel-(2S,4S,5R)-4-(Aminocarbonyl)-2-isobutyl-5-thien-2-ylpyrrolidine-2-carboxylic acid, tert-butyl ester

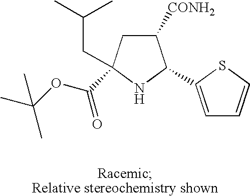

The title compound was prepared according to the method described for Intermediate 2 substituting 2-[N-(thien-2-ylmethylene)amino]-4-methylpentanoic acid, tert-butyl ester (Intermediate 4) in place of 2-[N-(1,3-thiazol-2-ylmethylene)amino]-4-methylpentanoic acid, tert-butyl ester.

¹H NMR (CDCl₃): δ 7.19 (dd, 1H), 7.03 (br.d, 1H), 6.95 (dd, 1H), 6.20 (br.s, 1H), 5.08 (br.s, 1H), 4.78 (d, 1H), 3.10 (m, 1H), 2.80 (v.br.s, 1H), 2.65 (dd, 1H), 2.12 (dd, 1H), 1.85 (dd, 1H), 1.74 (m, 1H), 1.62 (dd, 1H), 1.50 (s, 9H), 0.98 (d, 3H) and 0.92 (d, 3H).

### Intermediate 6

### rel-(2S,4S,5R)-4-aminocarbonyl)-1-(4-tert-butylbenzoyl)-2-isobutyl-5-thien-2-yl-pyrrolidine-2-carboxylic acid, tert-butyl ester

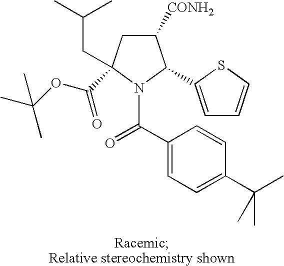

The title compound was prepared according to the method described for Intermediate 3 substituting rel-(2S,4S,5R)-4-(aminocarbonyl)-2-isobutyl-5-thien-2-ylpyrrolidine-2-carboxylic acid, tert-butyl ester (Intermediate 5) in place of rel-(2S,4S,5R)-4-(aminocarbonyl)-2-isobutyl-5-(1,3-thiazol-2-yl)-pyrrolidine-2-carboxylic acid, tert-butyl ester.

MS calcd for (C₂₉H₄₀N₂O₄S+H)⁺: 513. Found: (M+H)⁺=513

¹H NMR (CD₃OD): 7.26 (d, 2H), 7.07 (d, 1H), 6.96 (d, 2H), 6.87 (br s, 1H), 6.68 (m, 1H), 5.61 (d, 1H), 3.67 (m, 1H), 2.87 (t, 1H), 2.20 (m, 2H), 2.11-1.93 (m, 2H), 1.59 (s, 9H), 1.28 (s, 9H) and 1.09 (d, 6H). Amide protons exchanged with solvent.

### Intermediate 7

### rel-(2S,4S,5R)- and rel-(2S,4R,5R)-4-Cyano-2-isobutyl-5-(1,3-thiazol-2-yl)pyrrolidine-2-carbo-xylic acid, tert-butyl ester

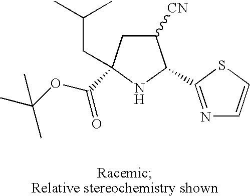

The title compound was prepared according to the method described for Intermediate 2 substituting acrylonitrile in place of acrylamide. The title compound was isolated as a 50:50 mixture of the rel-(2S,4S,5R)- and rel-(2S,4R,5R)-epimers.

MS calcd for (C₁₇H₂₅N₃O₂S+H)⁺: 336. Found: (M+H)⁺=336.

### Intermediate 8

### rel-(2S,4S,5R)- and rel-(2S,4R,5R)-4-Cyano-2-isobutyl-1-(4-tert-butylbenzoyl)-5-(1,3-thiazol-2-yl)pyrrolidine-2-carboxylic acid, tert-butyl ester

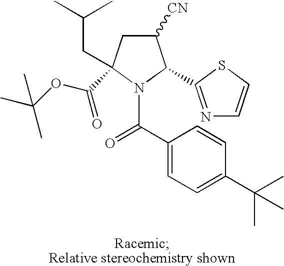

The title compound was prepared according to the method described for Intermediate 3, substituting a mixture of rel-(2S,4S,5R)- and rel-(2S,4R,5R)-4-cyano-2-isobutyl-5-(1,3-thiazol-2-yl)pyrrolidine-2-carboxylic acid tert-butyl ester (Intermediate 7) in place of rel-(2S,4S,5R)-4-(aminocarbonyl)-2-isobutyl-5-(1,3-thiazol-2-yl)pyrrolidine-2-carboxylic acid tert-butyl ester, and was isolated as a mixture together with the corresponding rel-(2S,4R,5R)-epimer.

MS calcd for (C₂₈H₃₇N₃O₃S+H))⁺: 496. Found (M+H)⁺=496.

### Intermediate 9

### rel-(2S,4S,5R)- and rel-(2S,4R,5R)-4-Acetyl-2-isobutyl-5-(1,3-thiazol-2-yl)pyrrolidine-2-carboxylic acid, tert-butyl ester

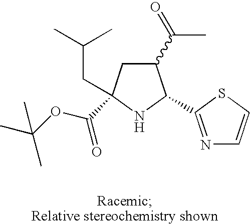

The title compound was prepared according to the method described for Intermediate 2 substituting 3-buten-2-one in place of acrylamide. The title compound was isolated as a mixture of the rel-(2S,4S,5R)- and rel-(2S,4R,5R)-epimers.

MS calcd for (C₁₈H₂₈N₂O₃S+H)⁺: 353. Found: (M+H)⁺=353.

### Intermediate 10

### rel-(2S,4R,5R)-4-Acetyl-2-isobutyl-1-(4-tert-butylbenzoyl)-5-(1,3-thiazol-2-yl)-pyrrolidine-2-carboxylic acid, tert-butyl ester

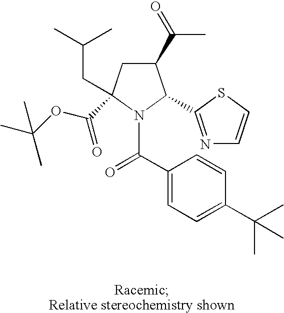

The title compound was prepared according to the method described for Intermediate 3, substituting a mixture of rel-(2S,4R,5R)- and rel-(2S,4S,5R)-4-acetyl-2-isobutyl-5-(1,3-thiazol-2-yl)pyrrolidine-2-carboxylic acid tert-butyl ester (Intermediate 9) in place of rel-(2S,4S,5R)-4-(aminocarbonyl)-2-isobutyl-5-(1,3-thiazol-2-yl)pyrrolidine-2-carboxylic acid tert-butyl ester. The title compound was shown by nOe NMR experiments to be the rel-(2S,4R,5R)-diastereoisomer.

MS calcd for (C₂₉H₄₀N₂O₄S+H)⁺: 513. Found (M+H)⁺=513.

### Intermediate 11

### rel-(2S,4S,5R)-2-Isobutyl-1-(4-tert-butylbenzoyl)-5-(1,3-thiazol-2-yl)-4-thiocarbamoyl-pyrrolidine-2-carboxylic acid, tert-butyl ester

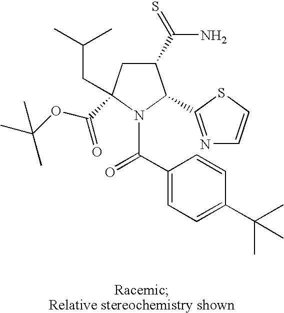

Lawesson's reagents (1.06 g, 2.62 mmol) was added to a solution of rel-(2S,4S,5R)-4-(aminocarbonyl)-1-(4-tert-butylbenzoyl)-2-isobutyl-5-(1,3-thiazol-2-yl)-pyrrolidine-2-carboxylic acid, tert-butyl ester (Intermediate 3; 1.30 g, 2.53 mmol) in anhydrous THF (50 mL) and the resulting mixture was heated under reflux for 18 hours, then evaporated. The resulting foam was chromatographed on silica gel using ethyl acetate-cyclohexane as eluent (gradient elution from 1:3 v/v to 1:2 v/v) to afford the title compound, a solid.

MS calcd for (C₂₈H₃₉N₃O₃S₂+H)⁺: 530. Found: (M+H)⁺=530.

¹H NMR (CDCl₃): δ 7.43 (1H, d), 7.38 (1H, br s), 7.24 (2H, d), 7.17 (1H, d), 7.16 (1H, br s), 7.02 (2H, d), 5.85 (1H, d), 4.04 (1H, m), 3.14 (1H, t), 2.49 (1H, dd), 2.28 (1H, dd), 2.16 (1H, dd), 1.96 (1H, m), 1.09 (3H, d), 1.07 (3H, d), 1.51 (9H, s) and 1.27 (9H, s).

### Intermediate 12

### rel-(2S,4S,5R)-2-Isobutyl-5-(1,3-thiazol-2-yl)pyrrolidine-2,4-dicarboxylic acid, 2-tert-butyl ester, 4-ethyl ester

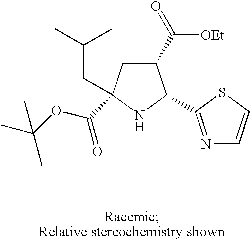

The title compound was prepared according to the method described for Intermediate 2 substituting ethyl acrylate in piece of acrylamide. The title compound was isolated as a gum.

MS calcd for (C₁₉H₃₀N₂O₄S+H)⁺: 383. Found: (M+H)⁺=383.

### Intermediate 13

### rel-(2S,4S,5R)-2-Isobutyl-1-(4-tert-butylbenzoyl)-5-(1,3-thiazol-2-yl)-pyrrolidine-2,4-dicarboxylic acid, 2-tert-butyl ester, 4-ethyl ester

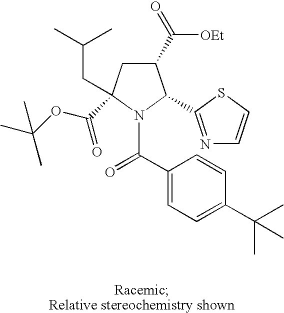

The title compound, a solid, was prepared according to the method described for Intermediate 3, substituting rel-(2S,4S,5R)-2-isobutyl-5-(1,3-thiazol-2-yl)pyrrolidine-2,4-dicarboxylic acid, 2-tert-butyl ester, 4-ethyl ester in place of rel-(2S,4S,5R)-4-(aminocarbonyl)-2-isobutyl-5-(1,3-thiazol-2-yl)pyrrolidine-2-carboxylic acid tert-butyl ester.

MS calcd for (C₃₀H₄₂N₂O₅S+H)⁺: 543. Found (M+H)⁺=543.

### Intermediate 14

### rel-(2S,4R,5R)-2-Isobutyl-1-(4-tert-butylbenzoyl)-4-hydrazinocarbonyl-5-(1,3-thiazol-2-yl)-pyrrolidine-2-carboxylic acid, tert-butyl ester

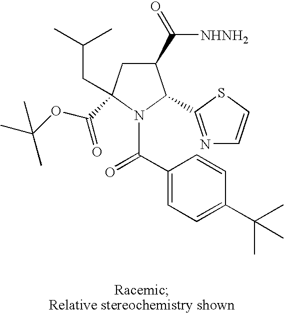

A solution of rel-(2S,4S,5R)-2-isobutyl-1-(4-tert-butylbenzoyl)-5-(1,3-thiazol-2-yl)-pyrrolidine-2,4-dicarboxylic acid, 2-tert-butyl ester, 4-ethyl ester (Intermediate 13; 2.00 g, 3.68 mmol), hydrazine hydrate (5 mL) and ethanol (50 mL) was heated under reflux for 4 hours. An additional aliquot of hydrazine hydrate (10 mL) was added and heating continued for an additional 6 hours. The mixture was concentrated and diluted with water (100 mL) and the resulting solid filtered and washed successively with water and cyclohexane. The solid was dissolved in ether, filtered and evaporated to afford the title compound as a mixture together with the rel-(2S,4S,5R)-diasteoisomer (ca. 65:35 w/w by NMR).

MS calcd for (C₂₈H₄₀N₄O₄S+H)⁺: 529. Found (M+H)⁺=529.

### Intermediate 15

### rel-(2S,4R,5R)- and rel-(2S,4S,5R)-2-Isobutyl-1-(4-tert-butylbenzoyl)-4-(ethoxymethylene-hydrazinocarbonyl)-5-(1,3-thiazol-2-yl)-pyrrolidine-2-carboxylic acid, tert-butyl ester

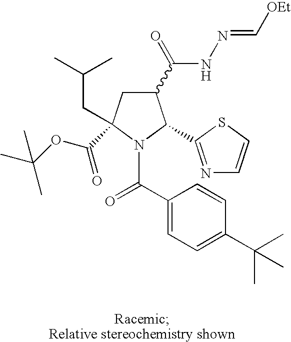

Further elution of the chromatography column from Example 23, Stage A using ethyl acetate-cyclohexane (3:1 v/v) as eluent afforded the title compound, a gum as an undefined mixture of diastereoisomers at the pyrrolidine C(4)-position.

MS calcd for (C₃₁H₄₄N₄O₅S+H)⁺: 585. Found (M+H)⁺=585.

### Intermediate 16

### 2-[N-(1,3-Thiazol-2-ylmethylene)amino]-3-phenylpropanoic acid, tert-butyl ester

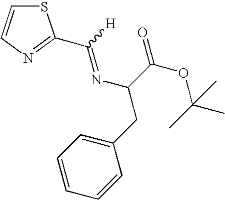

The title compound was prepared according to the method described for Intermediate 1, substituting phenylalanine tert-butyl ester in place of leucine tert-butyl ester.

MS calcd for (C₁₇H₂₀N₂O₂S+H)⁺: 316. Found (M+H)⁺=316.

### Intermediate 17

### rel-(2R,4S,5R)-2-Benzyl-4-(aminocarbonyl)-5-(1,3-thiazol-2-yl)pyrrolidine-2-carboxylic acid, tert-butyl ester

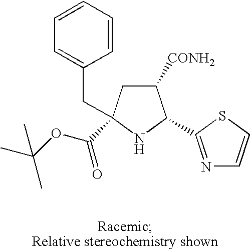

The title compound was prepared according to the method described for Intermediate 2 substituting 2-[N-(1,3-thiazol-2-ylmethylene)amino]-3-phenylpropanoic acid, tert-butyl ester (Intermediate 16) in place of 2-[N-(1,3-thiazol-2-ylmethylene)amino]-4-methylpentanoic acid, tert-butyl ester.

MS calcd for (C₂₀H₂₅N₃O₃S+H)⁺: 388. Found: (M+H)⁺=388

### Intermediate 18

### rel-(2R,4S,5R)-2-Benzyl-4-(aminocarbonyl)-1-(4-tert-butylbenzoyl)-5-(1,3-thiazol-2-yl)-pyrrolidine-2-carboxylic acid, tert-butyl ester

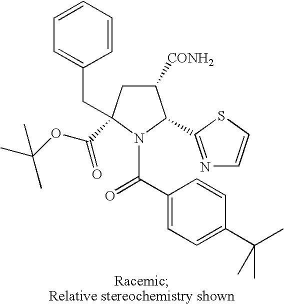

The title compound was prepared according to the method described for Intermediate 3, substituting rel-(2R,4S,5R)-2-benzyl-4-(aminocarbonyl)-5-(1,3-thiazol-2-yl)pyrrolidine-2-carboxylic acid, tert-butyl ester (Intermediate 17) in place of 2-[N-(1,3-thiazol-2-ylmethylene)amino]-4-methylpentanoic acid, tert-butyl ester.

MS calcd for (C₃₁H₃₇N₃O₄S+H)⁺: 548. Found: A+H)⁺=548

### Intermediate 19

### rel-(2S,4S,5R)-2-Isobutyl-4-(1-methyl-1H-tetrazol-5-yl)-5-(1,3-thiazol-2-yl)-pyrrolidine-2-carboxylic acid, tert-butyl ester

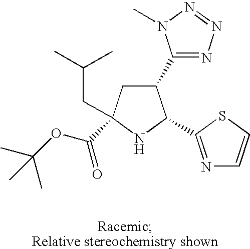

The title compound was prepared by analogy with the method described for Intermediate 2, substituting 1-methyl-5-vinyl-1H-tetrazole (J. Org. Chem., 1972, 37, 348) for acrylamide.

MS calcd for (C₁₈H₂₈N₆O₂S+H)⁺: 392. Found: (M+H)⁺=392.

### Intermediate 20

### rel-(2S,4S,5R)-2-Isobutyl-4-(benzothiazol-2-yl)-5-(1,3-thiazol-2-yl)-pyrrolidine-2-carboxylic acid, tert-butyl ester

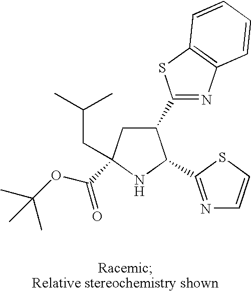

The title compound was prepared by analogy with the method described for Intermediate 2, substituting 2-vinylbenzothiazole (Eur. J. Med Chem., 1993, 28, 201) for acrylamide

MS calcd for (C₂₃H₂₉N₃O₂S₂+H)⁺: 444. Found: (M+H)⁺=444.

### Intermediate 21

### rel-(2S,4S,5R)-2-Isobutyl-4-(benzoxazol-2-yl)-5-(1,3-thiazol-2-yl)-pyrrolidine-2-carboxylic acid, tert-butyl ester

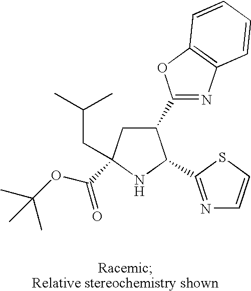

The tide compound was prepared by analogy with the method described for Intermediate 2, substituting 2-vinylbenzoxazole (J. Org. Chem., 1993, 58, 7009) for acrylamide

MS calcd for (C₂₃H₂₉N₃O₃S+H)⁺: 428. Found: (M+H)⁺=428.

### Intermediate 22

### rel-(2S,4S,5R)-4-(Aminocarbonyl)-1-(4-tert-butylbenzoyl)-2-isobutyl-5-pyridin-2-yl-pyrrolidine-2-carboxylic acid, tert-butyl ester

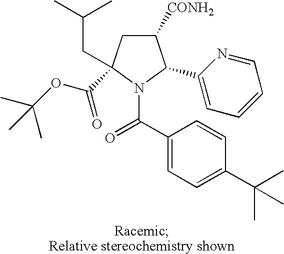

The title compound, a solid, was prepared by sequentially following procedures analogous to those described for Intermediates 1, 2 and 3 (above), but utilising pyridine-2-carboxaldehyde in place of 1,3-thiazole-2-carboxaldehyde.

MS calcd for (C₃₀H₄₁N₃O₄+H)⁺: 508. Found: (M+H)⁺=508.

¹H NMR (CDCl₃): δ 8.01 (1H, d), 7.97 (1H, d), 7.54 (1H, dt), 7.09 (2H, d), 6.98 (1H, dd), 6.84 (2H, d), 5.67 (1H, br), 5.37 (1H, d), 5.18 (1H, br), 3.67 (1H, ddd), 2.92 (1H, t), 2.28 (2H, m), 2.11 (1H, dd), 1.98 (1H, m), 1.59 (9H, s), 1.21 (9H, s) and 1.10 (6H, d).

### Intermediate 23

### Enantiomer A Derived from rel-(2S,4S,5R)-4-(aminocarbonyl)-2-isobutyl-5-(1,3-thiazol-2-yl)pyrrolidine-2-carboxylic acid, tert-butyl ester

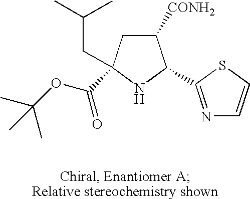

Stage A: A solution of (−)di-O,O′-p-tolyl-L-tartaric acid (2.74 g, 7.08 mmol) in dichloromethane (40 mL) was added to a solution of rel-(2S,4S,5R)-4-(aminocarbonyl)-2-isobutyl-5-(1,3-thiazol-2-yl)pyrrolidine-2-carboxylic acid, tert-butyl ester (Intermediate 2; 2.50 g, 7.08 mmol) in dichloromethane (200 mL). The resulting solution was allowed to stand overnight in a stoppered flask and the resulting crystalline solid filtered off, washed sparingly with dichloromethane and dried under vacuum to afford the tartrate salt of the title compound (3.75 g).

Stage B: A sample of the salt from Stage A (3.721 g) was added to a solution of sodium bicarbonate (4.00 g) in water (100 mL) and the resultant mixture was then extracted with dichloromethane (2×50 mL). The dichloromethane solution was dried by passage through a hydrophobic frit, then evaporated to afford a solid (1.07 g). Analytical HPLC using a Chiralpak AD chromatography column using heptane-ethanol (70:30 v/v) as eluent showed this solid to be the 1^{st} eluting enantiomer of the title compound (Enantiomer A; >99% ee; retention time 4.4 minutes), identical by 1H NMR to the racemic compound described in Intermediate 2.

Chiral analytical HPLC of the corresponding racemate (Intermediate 2) shows two peaks with retention times 4.4 and 10.9 minutes for Enantiomers A and B respectively under identical chiral HPLC conditions.

### Intermediate 24

### Enantiomer A of rel-(2S,4S,5R)-2-isobutyl-4-(3-methyl-1,2,4-oxadiazol-5-yl)-5-(1,3-thiazol-2-yl)pyrrolidine-2-carboxylic acid, tert-butyl ester

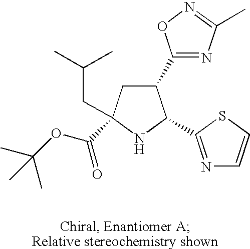

A mixture of Enantiomer A derived from rel-(2S,4S,5R)-4-(aminocarbonyl)-2-isobutyl-5-(1,3-thiazol-2-yl)pyrrolidine-2-carboxylic acid, tert-butyl ester (Intermediate 23; 2.98 g, 8.44 mmol) and (1,1-dimethoxyethyl)dimethylamine (20 mL) was heated at 120° C. for 2 hours then cooled and concentrated. The residue was dissolved in dioxan (14 mL) and acetic acid (14 mL) and hydroxylamine hydrochloride (0.82 g, 11.8 mmol) and aqueous sodium hydroxide (2M, 3 mL) were added. The mixture was heated at 90° C. for 2.5 hours, cooled and evaporated and the resulting material purified by chromatography on silica gel using cyclohexane-ethyl acetate (7:1 v/v) as eluent to afford Enantiomer A of the title compound, an oil.

¹H NMR (CD₃OD): δ 7.50 (1H, d), 7.40 (1H, d), 5.05 (1H, d), 4.10 (1H, m), 2.90 (1H, m), 2.40 (1H, m), 2.20 (3H, s), 1.80 (3H, m), 1.50 (9H, s), 1.00 (3H, s) and 0.90 (3H, s). Pyrrolidine proton exchanges with solvent.

### Intermediate 25

### Enantiomer A of rel-(2S,4R,5R)-2-isobutyl-4-(3-methyl-1,2,4-oxadiazol-5-yl)-5-(1,3-thiazol-2-yl)pyrrolidine-2-carboxylic acid, tert-butyl ester

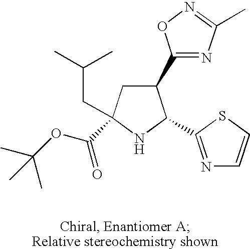

A mixture of Enantiomer A of rel-(2S,4S,5R)-2-isobutyl-4-(3methyl-1,2,4-oxadiazol-5-yl)-5-(1,3-thiazol-2-yl)pyrrolidine-2-carboxylic acid, tert-butyl ester (Intermediate 24; 1.00 g, 2.55 mmol) and methanolic sodium hydroxide (0.1M; 25.5 mL, 2.55 mmol; prepared from 2M aqueous sodium hydroxide diluted with methanol) was stirred at room temperature for 72 hours then concentrated. The residue was dissolved in dichloromethane, washed with dilute hydrochloric acid and brine, dried (Na₂SO₄) and evaporated to afford Enantiomer A of the title compound, an oil. This was shown by ¹H NMR spectroscopy and HPLC to be the rel-(2S,4R,5R)-diastereoisomer, the pyrrolidine C(4) epimer of the starting material.

¹H NMR (CD₃OD): δ 7.90 (1H, d), 7.70 (1H, d), 5.20 (1H, d), 3.95 (1H, m), 3.15 (1H, m), 2.55 (3H, s), 2.40 (1H, m), 2.15 (1H, m), 1.90 (2H, m), 1.70 (9H, s) and 1.15 (6H, d), Pyrrolidine proton exchanges with solvent.

### Intermediate 26

### rel-(2S,4R,5R)-4-Cyano-2-isobutyl-5-(1,3-thiazol-2-yl)pyrrolidine-2-carb-oxylic acid, tert-butyl ester

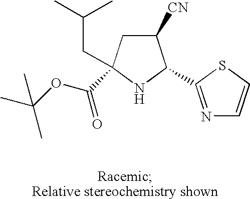

The mixture of diastereoisomers of rel-(2S,4S,5R)- and ref-(2S,4R,5R)-4-Cyano-2-isobutyl-5-(1,3-thiazol-2-yl)pyrrolidine-2-carboxylic acid, tert-butyl ester (Intermediate 7) was chromatographed on silica gel using initially cyclohexane-ethyl acetate (10:1 v/v) as eluent to afford the title compound, the rel-(2S,4R,5R)-diastereoisomer as a solid.

MS calcd for (C₁₇H₂₅N₃O₂S+H)⁺: 336. Found: (M+H)⁺=336.

¹H NMR (CD₃OD): δ 7.65 (1H, d), 7.40 (1H, d), 4.70 (1H, d), 3.15 (1H, m), 2.80 (1H, dd), 2.00 (1H, dd), 1.80 (1H, m), 1.60 (2H, m), 1.30 (9H, s) and 0.85 (6H, m). The pyrrolidine NH proton exchanges with the solvent.

Continued elution of the chromatography column with cyclohexane-ethyl acetate (7:3 v/v) as eluent afforded the corresponding rel-(2S,4S,5R)-diastereoisomer, described as Intermediate 27 (below).

### Intermediate 27

### rel-(2S,4S,5R)-4-Cyano-2-isobutyl-5-(1,3-thiazol-2-yl)pyrrolidine-2-carboxylic acid, tert-butyl ester

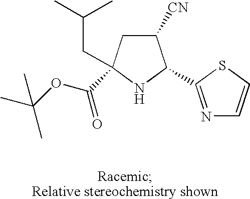

Continued elution of the chromatography column described in Intermediate 26, using cyclohexane-ethyl acetate (7:3 v/v) as eluent afforded the title compound, the rel-(2S,4S,5R)-diastereoisomer.

MS calcd for (C₁₇H₂₅N₃O₂S+H)⁺: 336. Found. (M+H)⁺=336.

¹H NMR (CD₃OD): δ 7.80 (1H, d), 7.55 (1H, d), 4.90 (1H, d), 3.70 (1H, m), 2.80 (1H, dd), 2.30 (1H, d), 1.75 (3H, m), 1.30 (9H, s), 0.95 (6H, m). The pyrrolidine NH proton exchanges with the solvent.

### Intermediate 28

### rel-(2S,4R,5R)-2-Isobutyl-4-(5-methyl-1,2,4-oxadiazol-3-yl)-5-(1,3-thiazol-2-yl)pyrrolidine-2-carboxylic acid, tert-butyl ester

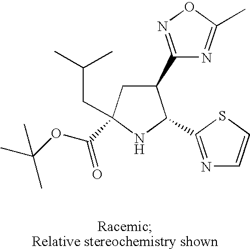

Hydroxylamine hydrochloride (0.334 g, 4.81 mmol) was added to a solution of rel-(2S,4R,5R)-4-cyano-2-isobutyl-5-(1,3-thiazol-2-yl)pyrrolidine-2-carboxylic acid, tert-butyl ester (Intermediate 26; 1.00 g, 2.99 mmol) in ethanol (35 mL). Potassium hydroxide (0.232 g, 4.15 mmol) was added and the mixture heated at reflux for 3 hours. The mixture was allowed to cool to room temperature overnight, then concentrated. N,N-dimethylacetamide dimethylacetal (25 mL) was added and the mixture heated at 100° C. for 5 hours. The mixture was cooled and concentrated, suspended in ethyl acetate and washed with dilute aqueous hydrochloric acid (2M) and water. The ethyl acetate solution was dried (Na₂SO₄) and evaporated. The resulting oil was chromatographed on silica gel using a gradient elution from cyclohexane to cyclohexane-ethyl acetate (2:3 v/v) to afford the title compound, an oil.

MS calcd for (C₁₉H₂₈N₄O₃S+H)⁺: 393. Found: (M+H)⁺=393.

¹H NMR (CD₃OD): δ 7.50 (1H, d), 7.30 (1H, d), 4.70 (1H, d), 3.35 (1H, m), 2.70 (1H, dd), 2.45 (3H, s), 2.05 (1H, t), 1.80 (1H, m), 1.60 (2H, m), 1.35 (9H, s) and 0.80 (6H, d). The pyrrolidine NH proton exchanges with the solvent.

### Intermediate 29

### rel-(2S,4S,5R)-2-Isobutyl-4-(5-methyl-1,2,4-oxadiazol-3-yl)-5-(1,3-thiazol-2-yl)pyrrolidine-2-carboxylic acid, tert-butyl ester

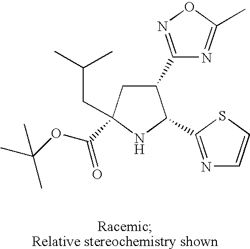

The title compound, an oil, was prepared from rel-(2S,4S,5R)-4-Cyano-2-isobutyl-5-(1,3-thiazol-2-yl)pyrrolidine-2-carboxylic acid, tert-butyl ester (Intermediate 27) in an analogous manner to that described for the preparation of Intermediate 28.

MS calcd for (C₁₉H₂₈N₄O₃S+H)⁺: 393. Found: (M+H)⁺=393.

¹H NMR (CD₃OD): δ 7.45 (1H, d), 7.30 (1H, d), 4.90 (1H, d), 3.90 (1H, dd), 2.75 (1H, dd), 2.30 (3H, s), 2.20 (1H, dd), 1.70 (3H, m), 1.45 (9H, s), 0.95 (3H, d) and 0.85 (3H, d). The pyrrolidine NH proton exchanges with solvent.

### Intermediate 30

### rel-(2S,4S,5R)-4-(Aminocarbonyl)-2-isobutyl-5-(benzothiazol-2-yl)pyrrolidine-2-carboxylic acid, tert-butyl ester

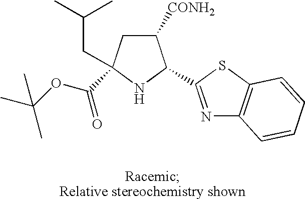

The title compound, a solid, was prepared by sequentially following procedures analogous to those described for Intermediates 1 and 2, but utilising benzothiazole-2-carboxaldehyde in place of 1,3-thiazole-2-carboxaldehyde.

MS calcd for (C₂₁H₂₉N₃O₃S+H)⁺: 403. Found: (M+H)⁺=403.

¹H NMR (CDCl₃): δ 7.90 (1H, d), 7.85 (1H, d), 7.50(1H, m), 7.35 (1H, m), 6.10 (1H, br), 5.20 (1H, br), 4.90 (1H, d), 3.40 (1H, q), 3.20 (1H, br), 2.80 (1H, dd), 2.10 (1H, dd), 1.80 (2H, m), 1.60 (1H, dd), 1.50 (9H, s), 1.00 (3H, dd) and 0.90 (3H, dd).

### Intermediate 31

### Enantiomer A Derived from rel-(2S,4S,5R)-4-(aminocarbonyl)-2-isobutyl-5-(benzothiazol-2-yl)pyrrolidine-2-carboxylic acid, tert-butyl ester

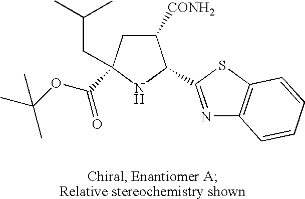

Stage A: A solution of (−)-di-O,O′-p-tolyl-L-tartaric acid (10.58 g, 27.4 mmol) in dichloromethane (120 mL) was added to a solution of rel-(2S,4S,5R)-4-(aminocarbonyl)-2-isobutyl-5-(benzothiazol-2-yl)pyrrolidine-2-carboxylic acid, tert-butyl ester (Intermediate 30; 6.00 g, 13.7 mmol) in dichloromethane (60 mL). The resulting solution was allowed to stand overnight in a stoppered flask and the resulting crystalline solid filtered off, washed sparingly with dichloromethane and dried under vacuum to afford the tartrate salt of the title compound (3.75 g).

Stage B: The salt from Stage A (9.195 g) was added to a solution of sodium bicarbonate (4.00 g) in water (100 mL) and the resultant mixture was then extracted with dichloromethane (2×100 mL). The dichloromethane solution was dried by passage through a hydrophobic frit, then evaporated to afford a solid (2.60 g). Analytical HPLC using a Chiralpak AD chromatography column using heptane-ethanol (80:20 v/v) as eluent showed this solid to be the 1^{st} eluting enantiomer of the title compound (Enantiomer A; 96% ee; retention time 5.40 minutes), identical by 1H NMR to the racemic compound described in Intermediate 30.

Chiral analytical HPLC of the corresponding racemate (Intermediate 30) shows two peaks with retention times 5.46 and 8.48 minutes for Enantiomers A and B respectively under identical chiral HPLC conditions.

### Intermediate 32

### Enantiomer A of rel-(2S,4S,5R)-4-(aminocarbonyl)-2-isobutyl-1-(4-tert-butylbenzoyl)-5-(benzothiazol-2-yl)pyrrolidine-2-carboxylic acid, tert-butyl ester

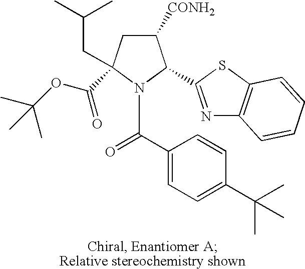

Enantiomer A derived from rel-(2S,4S,5R)-4-(aminocarbonyl)-2-isobutyl-5-(benzothiazol-2-yl)pyrrolidine-2-carboxylic acid, tert-butyl ester (Intermediate 31) was treated with 4-tert-butylbenzoyl chloride in a manner analogous to that described for Intermediate 3. The crude product was purified by chromatography on silica gel using cyclohexane-ethyl acetate (1:1 v/v) as eluent to afford Enantiomer A of the title compound, a solid.

MS calcd for (C₃₂H₄₁N₃O₄S+H)⁺: 564. Found: (M+H)⁺=564.

¹H NMR (CD₃OD): δ 7.90 (1H, d), 7.70 (1H, d), 7.40 (2H, m), 7.20 (2H, d), 7.10 (2H, d), 5.90 (1H, d), 3.90 (1H, m), 3.10 (1H, t), 2.35 (1H, dd), 2.30-2.00 (3H, m), 1.60 (9H, s), 1.20 (9H, s) and 1.10 (6H, dd). Amide protons exchange with solvent.

### Intermediate 33

### 5-Chloro-3-(4-fluorophenyl)-1,2,4-oxadiazole

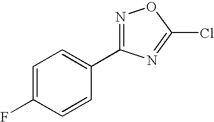

Ethyl chloroformate (1.23 mL, 12.97 mmol) was added to a stirred solution of 4-fluorophenylbenzamidoxime (2.00 g, 12.97 mmol) in anhydrous pyridine (20 mL). The mixture was heated at 120° C. for 4 hours, cooled and evaporated. The residue was dissolved in pyridine (1.29 mL) and phosphorus oxychloride (13 mL) was added. The mixture was heated at reflux for 1 hour, cooled and diluted with saturated aqueous sodium bicarbonate solution, then extracted with ethyl acetate. The ethyl acetate extracts were dried (Na₂SO₄) and concentrated and the resulting solid purified by chromatography on silica gel using a gradient elution from cyclohexane-ethyl acetate (5:1 v/v) to cyclohexane-ethyl acetate (3:1 v/v) to afford the title compound, a solid.

¹H NMR (CDCl₃): δ 8.07 (2H, m) and 7.20 (2H, t).

### Intermediate 34

### 3-(4-Fluorophenyl)-5-vinyl-1,2,4-oxadiazole

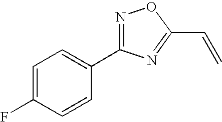

Zinc bromide (oven dried at 100° C.; 2.37 g, 10.5 mmol) was added to a stirred, −78° C. solution of vinylmagnesiumn bromide (1.0 M in THF; 10.5 mL, 10.5 mmol) in dry THF under nitrogen. The reaction mixture was stirred at −78° C. for 1 hour prior to the addition of palladium tetrakistriphenylphosphine (0.21 g) and 5-Chloro-3-(4-fluorophenyl)-1,2,4-oxadiazole (Intermediate 33; 1.45 g, 7.30 mmol). The mixture was heated at 50° C. for 6 hours, cooled and partitioned between saturated aqueous ammonium chloride solution and ethyl acetate. The organic solution was dried (Na₂SO₄) and evaporated and the residue subsequently purified by chromatography on silica gel using cyclohexane-ethyl acetate (19:1 v/v) as eluent to afford the title compound, a solid.

¹H NMR (CDCl₃): δ 8.11 (2H, m), 7.19 (2H, m), 6.77 (1H, dd), 6.60 (1H, d) and 6.10 (1H, d).

### Intermediate 35

### rel-(2S,4S,5R)-4-[3-(4-fluorophenyl)-1,2,4-oxadiazol-5-yl]-2-isobutyl-5-(1,3-thiazol-2-yl)pyrrolidine-2-carboxylic acid, tert butyl ester

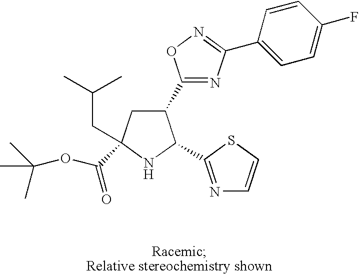

Lithium bromide (0.41 g, 4.72 mmol) was added to a solution of 3-(4fluorophenyl)-5-vinyl-1,2,4-oxadiazole (Intermediate 34; 0.89 g, 2.36 mmol) and 2-[N-(1,3-thiazol-2-ylmethylene)amino]-4-methylpentanoic acid, tert-butyl ester (0.66 g, 2.36 mmol) in anhydrous THF under nitrogen. The mixture was stirred for 5 minutes at 5° C. prior to the addition of triethylamine (0.65 mL, 4.72 mmol) and after a further 5 minutes, the solution was allowed to warm to room temperature and stirred for a further 2.5 hours before being diluted with saturated aqueous ammonium chloride. The mixture was extracted with ethyl acetate and the organic solution washed with water, dried (Na₂SO₄) and evaporated. The residue was chromatographed on silica gel using cyclohexane-ethyl acetate (5:1 v/v) as eluent to afford the title compound, a solid.

MS calcd for (C₂₄H₂₉FN₄O₃S+H)⁺: 473. Found: (M+H)⁺=473.

¹H NMR (CDCl₃): δ 7.49 (2H, m), 7.54 (1H, d), 7.15 (1H, d), 7.10 (2H, t), 5.08 (1H, d), 4.13 (1H, m), 3.37 (1H, br), 2.98 (1H, dd), 2.36 (1H, dd), 1.89-1.78 (2H, m), 1.73-1.65 (1H, m), 1.52 (9H, s), 1.12 (3H, d) and 0.95 (3H, d).

### Intermediate 36

### 3-Bromo-5-vinyl-1,2,4-thiadiazole

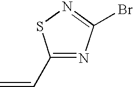

Oven dried zinc bromide (4.74 g, 21.0 mmol) was added to a cold (−78° C.) solution of vinyl magnesium bromide (1.0 M solution in THF, 21.0 mL) in dry THF (84 mL) under nitrogen. The reaction mixture was stirred at −78° C. for 1 hour then allowed to warm to room temperature over a further 1 hour. Palladium tetrakis triphenylphosphine (0.42 g) and 3-bromo-5-chloro-1,2,4-thiadiazole (2.90 g, 14.30 mmol) were added and the mixture was then heated at 50° C. under nitrogen for 20 hours. The reaction mixture was cooled and stirred vigorously during the addition of saturated aqueous ammonium chloride. The mixture was extracted with ethyl acetate and the organic solution dried (Na₂SO₄) and evaporated to afford an oil. This was purified by silica gel chromatography using cyclohexane-ethyl acetate (9:1 v/v) to afford the title compound as an oil.

¹H NMR (CDCl₃): δ 6.97 (1H, dd), 6.35 (1H, d) and 5.89 (1H, d).

### Intermediate 37

### rel-(2S,4R,5R)-4-[3-Bromo-1,2,4-thiadiazol-5-yl]-2-isobutyl-5-(1,3-thiazol-2-yl)pyrrolidine-2-carboxylic acid, tert butyl ester

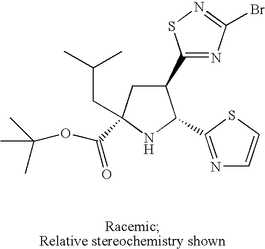

3-Bromo-5-vinyl-1,2,4-thiadiazole (Intermediate 36; 1.43 g, 7.48 mmol) was reacted with 2-[N-(1,3-thiazol-2-ylmethylene)amino]-4-methylpentanoic acid, tert-butyl ester (Intermediate 1; 2.11 g, 7.48 mmol) in a manner analogous to that described for Intermediate 35. The crude product so obtained was purified by chromatography on silica gel using cyclohexane-ethyl acetate (7:1 v/v) as eluent to afford the title compound, a gum.

MS calcd for (C₁₈H₂₅BrN₄O₂S₂+H)⁺: 473/475. Found: (M+H)⁺=473/475.

¹H NMR (CDCl₃): δ 7.73 (1H, d), 7.29 (1H, d), 4.76 (1H, d), 4.04 (1H, m), 3.11 (1H, br), 3.00 (1H, dd), 2.37 (1H, dd), 1.78-1.86 (2H, m), 1.74 (1H, m), 1.46 (9H, s), 0.97 (3H, d) and 0.94 (3H, d).

Continued elution of the chromatography column afforded the rel-(2S,4S,5R)-diastereoisomer, described further as Intermediate 38 (below).

### Intermediate 38

### rel-(2S,4S,5R)-4-[3-Bromo-1,2,4-thiadiazol-5-yl]-2-isobutyl-5-(1,3-thiazol-2-yl)pyrrolidine-2-carboxylic acid, tert butyl ester

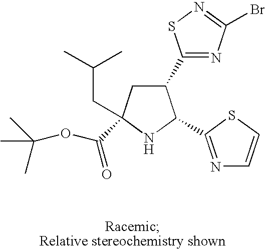

Continued elution of the chromatography column described in Intermediate 37 (above) afforded the title compound, a solid.

MS calcd for (C₁₈H₂₅BrN₄O₂S₂+H)⁺: 473/475. Found: (M+H)⁺=473/475.

¹H NMR (CDCl₃): δ 7.65 (1H, d), 7.20 (1H, d), 4.99 (1H, d), 4.31 (1H, q), 3.16 (1H, br), 2.96 (1H, dd), 2.39 (1H, dd), 1.55-1.85 (3H, m), 1.51 (9H, s), 0.99 (3H, d) and 0.94 (3H, d).

### Intermediate 39

### rel-(2S,4S,5R)-2-Isobutyl-4-(3-methyl-1,2,4-oxadiazol-5-yl)-5-thien-2-ylpyrrolidine-2-carboxylic acid, tert-butyl ester

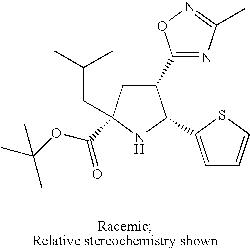

The title compound, an oil, was prepared according to the method described for Intermediate 24 substituting rel-(2S,4S,5R)-4-(aminocarbonyl)-2-isobutyl-5-thien-2-ylpyrrolidine-2-carboxylic acid, tert-butyl ester (Intermediate 5) in place of Enantiomer A derived from rel-(2S,4S,5R)-4-(aminocarbonyl)-2-isobutyl-5-(1,3-thiazol-2-yl)pyrrolidine-2-carboxylic acid, tert-butyl ester (Intermediate 23).

MS calcd for (C₂₀H₂₉N₃O₃S+H)⁺: 392. Found: (M+H)⁺=392.

¹H NMR (CDCl₃): δ 7.20 (1H, d), 6.80-6.90 (2H, m), 5.00 (1H, d), 4.00 (1H, m), 2.80 (1H, dd), 2.30-2.40 (1H, dd), 2.20 (3H, s), 1.70-1.85 (3H, m), 1.60 (9H, s), 1.05 (3H, d) and 0.95 (3H, d). Pyrrolidine proton exchanges with solvent.

### Intermediate 40

### rel-(2S,4R,5R-2-Isobutyl-4-(3-methyl-1,2,4-oxadiazol-5-yl)-5-thien-2-ylpyrrolidine-2-carboxylic acid, tert-butyl ester

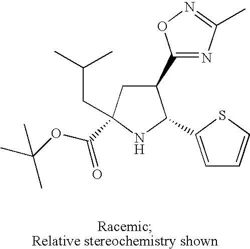

The title compound, an oil, was prepared according to the method described for Intermediate 25 substituting rel-(2S,4S,5R)-2-isobutyl-4-(3-methyl-1,2,4-oxadiazol-5-yl)-5-thien-2-ylpyrrolidine-2-carboxylic acid, tert-butyl ester (Intermediate 39) in place of Enantiomer A of rel-(2S,4S,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 24).

MS calcd for (C₂₀H₂₉N₃O₃S+H)⁺: 392. Found: (M+H)⁺=392.

¹H NMR (CDCl₃): δ 7.35 (1H, d), 7.00 (1H, d), 6.95 (1H, t), 4.80 (1H, d), 3.55 (1H, m), 2.90 (1H, dd), 2.35 (3H, s), 2.20-2.30 (1H, dd), 1.80 (2H, m), 1.70-1.80 (1H, m), 1.55 9(H, s) and 1.00 (6H, m). Pyrrolidine proton exchanges with solvent.

### Intermediate 41

### rel-(2S,4R,5R)-4-Cyano-2-Isobutyl-4-methyl-5-(1,3-thiazol-2-yl)pyrrolidine-2-carboxylic acid, tert-butyl ester

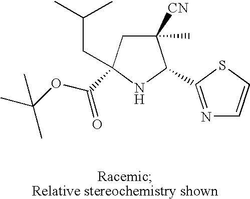

The title compound, a solid, was prepared according to the method described for Intermediate 2 substituting 2-methylacrylonitrile in place of acrylamide and was subsequently purified by chromatography on silica gel using cyclobexane-ethyl acetate (10:1 v/v) as eluent.

MS calcd for (C₁₈H₂₇N₃O₂S+H)⁺: 350. Found: (M+H)⁺=350

¹H NMR (CD₃OD): δ 7.85 (1H, d), 7.60 (1H, d), 4.95 (1H, s), 2.75 (1H, d), 2.45 (1H, d), 1.70-1.90 (3H, m), 1.50 (9H, s) and 1.00 (9H, m). Pyrrolidine proton exchanges with solvent.

### Intermediate 42

### rel-(2S,4R,5R)-2-Isobutyl-4-methyl-4-(5-methyl-1,2,4-oxadiazol-3-yl)-5-(1,3-thiazol-2-yl)pyrrolidine-2-carboxylic acid, tert-butyl ester

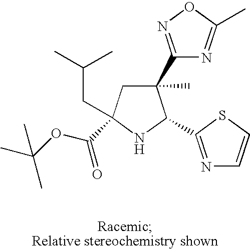

The title compound, a solid, was prepared in a similar manner to that described for Intermediate 28, but substituting rel-(2S,4R,5R)-4-cyano-2-Isobutyl-4-methyl-5-(1,3-thiazol-2-yl)pyrrolidine-2-carboxylic acid, tert-butyl ester (Intermediate 41) in place of rel-(2S,4R,5R)-4-cyano-2-Isobutyl-5-(1,3-thiazol-2-yl)pyrrolidine-2-carboxylic acid, tert-butyl ester.

MS calcd for (C₂₀H₃₀N₄O₃S+H)⁺: 407. Found: (M+H)hu +=407.

¹H NMR (CD₃OD): δ 7.75 (1H, d), 7.50 (1H, d), 5.10 (1H, s), 2.45-32.65 (2H, dd), 2.60 (3H, s), 1.70-1.85 (3H, m), 1.55 (9H, s), 1.05 (3H, s) and 0.95-1.00 (6H, dd). The pyrrolidine NH proton exchanges with the solvent.

### Intermediate 43

### rel-(2S,4R,5R)-4-Cyano-2-isobutyl-5-thien-2-ylpyrrolidine-2-carboxylic acid, tert-butyl ester

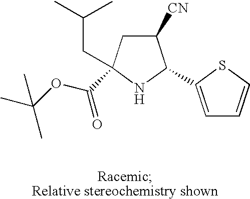

Acrylonitrile (1.17 mL, 0.0178 mol) was added to a cooled (0° C.) solution of 2-[N-(thien-2-ylmethylene)amino]-4-methylpentanoic acid, tert-butyl ester (Intermediate 4; 4.00 g, 0.0142 mol) in dry THF (20 mL). Lithium bromide (2.47 g,0.0284 mol) was added and the mixture stirred at 0° C. for 5 minutes prior to the addition of triethylamine (2.47 mL, 0.0177 mol). The resulting mixture was stirred at room temperature overnight then partitioned between ethyl acetate and saturated aqueous ammonium chloride solution. The ethyl acetate solution was washed with brine, dried (Na2SO4) and evaporated to afford an oil. This oil was purified by chromatography on silica gel using cyclohexane-ethyl acetate (11:1 v/v) as eluent to afford the title compound, a solid.

MS calcd for (C₁₈H₂₆N₂O₂S+H)⁺: 335. Found: (M+H)⁺=335.

¹H NMR (CD₃OD): δ 7.25 (1H, d), 7.05 (1H, d), 6.90 (1H, dd), 4.55 (1H, d), 2.80-2.90 (1H, m), 2.70-2.80 (1H, dd), 2.00 (1H, t), 1.65-1.70 (2H, m), 1.60 (1H, m), 1.40 (9H, s) and 0.85 (6H, m). Pyrrolidine proton exchanges with solvent.

Continued elution of the chromatography column afforded the corresponding rel-(2S,4S,5R)-diastereoisomer, described as Intermediate 44 (below).

### Intermediate 44

### rel-(2S,4S,5R)-4-Cyano-2-isobutyl-5-thien-2-ylpyrrolidine-2-carboxylic acid, tert-butyl ester

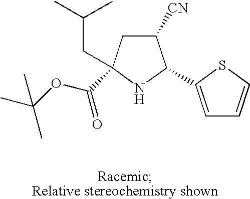

Continued elution of the chromatography column described in Intermediate 43, using cyclohexane-ethyl acetate (11:1 v/v) as eluent afforded the title compound, an oil.

MS calcd for (C₁₈H₂₆N₂O₂S+H)⁺: 335. Found: (M+H)⁺=335.

¹H NMR (CD₃OD): δ 7.35 (1H, d), 7.15 (1H, d), 7.05 (1H, dd), 4.70 (1H, d), 3.50 (1H, m), 2.70-2.75 (1H, dd), 2.20-2.30 (1H, dd), 1.70-1.80 (2H, m), 1.60 (1H, m), 1.50 (9H, s), 0.95 (3H, d), 0.90 (3H, d). Pyrrolidine proton exchanges with solvent.

### Intermediate 45

### rel-(2S,4R,5R)-2-Isobutyl-4-(5-methyl-1,2,4-oxadiazol-3-yl)-5-thien-2-ylpyrrolidine-2-carboxylic acid, tert-butyl ester

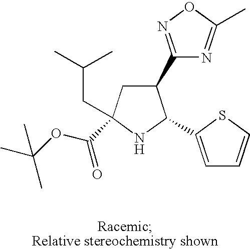

The title compound, a solid, was prepared from rel-(2S,4R,5R)-4-Cyano-2-isobutyl-5-thien-2-ylpyrrolidine-2-carboxylic acid, tert-butyl ester (Intermediate 43) in an analogous manner to that described for the preparation of Intermediate 28.

MS calcd for (C₂₀H₂₉N₃O₃S+H)⁺: 392. Found: (M+H)⁺=392.

¹H NMR (CD₃OD): δ 7.20 (1H, d), 6.80 (2H, m), 4.60 (1H, d), 3.25 (1H, m), 2.75 (1H, dd), 2.45 (3H, s), 2.10 (1H, t), 1.60-1.70 (3H, m), 1.40 (9H, s) and 0.80-0.90 (6H, m). Pyrrolidine proton exchanges with the solvent

### Intermediate 46

### rel-(2S,4S,5R)-2-Isobutyl-4-(5-methyl-1,2,4-oxadiazol-3-yl)-5-thien-2-ylpyrrolidine-2-carboxylic acid, tert-butyl ester

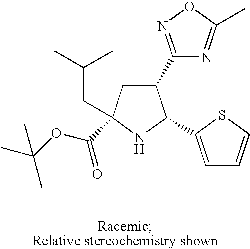

The title compound, a solid, was prepared from rel-(2S,4S,5R)-4-Cyano-2-isobutyl-5-thien-2-ylpyrrolidine-2-carboxylic acid, tert-butyl ester (Intermediate 44) in an analogous manner to that described for the preparation of Intermediate 28.

MS calcd for (C₂₀H₂₉N₃O₃S+H)⁺: 392. Found: (M+H)⁺=392.

¹H NMR (CD₃OD): δ 7.20 (1H, d), 6.85 (2H, m), 4.90 (1H, d), 3.85 (1H, q), 2.75 (1H, dd), 2.45 (3H, s), 2.25-2.35 (1H, dd), 1.75-1.85 (3H, m), 1.55 (9H, s), 1.05 (3H, d) and 0.95 (3H, d). Pyrrolidine proton exchanges with the solvent.

### Intermediate 47

### rel-(2S,4S,5R)-2-Isobutyl-4-(3-methyl-1,2,4-oxadiazol-5-yl)-5-pyridin-3-ylpyrrolidine-2-carboxylic acid, tert-butyl ester

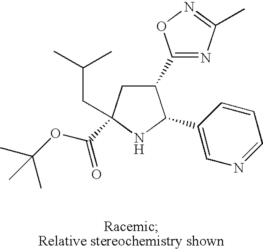

The title compound, an oil, was prepared by sequentially following procedures analogous to those described for Intermediates 1, 2 and 24, but utilising pyridine-3-carboxaldehyde in place of 1,3-thiazole-2-carboxaldehyde.

MS calcd for (C₂₁H₃₀N₄O₃+H)⁺: 387. Found: (M+H)⁺=387.

¹H NMR (CD₃OD): δ 8.40 (1H, s), 8.35 (1H, d), 7.65 (1H, d), 7.30 (1H, dd), 4.85 (1H, d), 4.10 (1H, m), 2.80 (1H, dd), 2.40 (1H, dd), 2.15 (3H, s), 1.80-1.90 (3H, m), 1.60 (9H, s), 1.05 (3H, d) and 0.95 (3H, d). Pyrrolidine proton exchanges with solvent.

### Intermediate 48

### 2-[N-(1,3-Thiazol-4-ylmethylene)amino]-4-methylpentanoic acid, tert-butyl ester

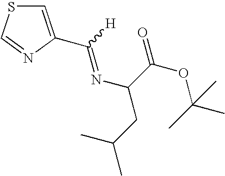

A stirred mixture of 2-amino-4-methyl-pentanoic acid tert-butyl ester, hydrochloride salt (3.58 g, 16.0 mmol), 1,3-thiazole-4-carboxaldehyde (1.81 g, 16.0 mmol) and triethylamine (2.23 mL, 16.0 mmol) in dichloromethane (25 mL) was heated under reflux under nitrogen for 4.5 hours. The reaction mixture was allowed to cool to room temperature, washed twice with water, dried over Na₂SO₄ and evaporated to give the title compound as a solid. ¹H NMR (CDCl₃): δ 8.85 (1H, d), 8.50 (1H, s), 8.00 (1H, d), 4.00 (1H, dd), 1.75-1.90 (2H, m), 1.50-1.60 (1H, m), 1.45 (9H, s), 0.95 (3H, d) and 0.90 (3H, d).

### Intermediate 49

### rel-(2S,4R,5R)-4Cyano-2-isobutyl-5-(1,3-thiazol-4-yl)pyrrolidine-2-carboxylic acid, tert-butyl ester

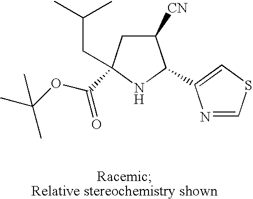

The title compound, a solid, was prepared by reaction of acrylonitrile with 2-[N-(1,3-thiazol-4-ylmethylene)amino]-4-methylpentanoic acid, tert-butyl ester (Intermediate 48) in an analogous manner to that described for the preparation of Intermediate 43.

MS calcd for (C₁₇H₂₅N₃O₂S+H)⁺: 336. Found: (M+H)⁺=336.

¹H NMR (CD₃OD): δ 8.90 (1H, s), 7.55 (1H, s), 4.45 (1H, d), 3.00-3.10 (1H, dd), 2.70 (1H, dd), 2.05 (1H, dd), 1.60-1.70 (3H, m), 1.40 (9H, s), 0.85 (3H, d) and 0.80 (3H, d). Pyrrolidine proton exchanges with solvent.

### Intermediate 50

### rel-(2S,4R,5R)-2-Isobutyl-4-(5-methyl-1,2,4-oxadiazol-3-yl)-5-(1,3-thiazol-4-yl)pyrrolidine-2-carboxylic acid, tert-butyl ester

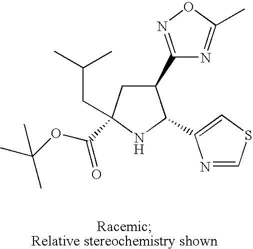

The title compound, an oil, was prepared from rel-(2S,4R,5R)-4-cyano-2-isobutyl-5-(1,3-thiazol-4-yl)pyrrolidine-2-carboxylic acid, tert-butyl ester (Intermediate 49) in an analogous manner to that described for the preparation of Intermediate 28.

MS calcd for (C₁₉H₂₈N₄O₃S+H)⁺: 393. Found: (M+H)⁺=393.

¹H NMR (CD₃OD): δ 8.85 (1H, s), 7.35 (1H, s), 4.50 (1H, d), 3.40 (1H, m), 2.70 (1H, dd), 2.45 (3H, s), 2.10 (1H, dd), 1.60-1.75 (3H, m), 1.40 (9H, s) and 0.80-0.90 (6H, d). Pyrrolidine proton exchanges with solvent.

### Intermediate 51

### rel-(2S,4S,5R)-2-Isobutyl-1-(3-methoxy-4-tert-butylbenzoyl)-5-(1,3-thiazol-2-yl)-pyrrolidine-2,4-dicarboxylic acid, 2-tert-butyl ester, 4-ethyl ester

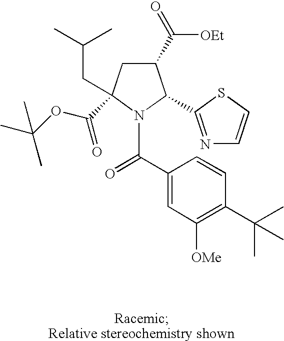

3-Methoxy-4-tert-butylbenzoyl chloride (2.89 g, 12.4 mmol) was added to a stirred solution of rel-(2S,4S,5R)-2-isobutyl-5-(1,3-thiazol-2-yl)pyrrolidine-2,4-dicarboxylic acid, 2-tert-butyl ester, 4-ethyl ester (Intermediate 12; 3.92 g, 10.24 mmol) and triethylamine (1.78 mL, 12.82 mmol) in anhydrous dichloromethane (100 mL). The mixture was stirred at room temperature for 48 hours then washed with saturated aqueous sodium bicarbonate solution. The organic solution was dried (Na₂SO₄) and evaporated and the residue purified by chromatography on silica gel using cyclohexane-ethyl acetate (7:1 v/v) as eluent to afford the title compound, a gum.

MS calcd for (C₃₁H₄₄N₂O₆S+H)⁺: 573. Found (M+H)⁺=573.

¹H NMR (CDCl₃): δ 7.48 (1H, d), 7.20 (1H, d), 7.16 (1H, d), 6.78 (1H, dd), 6.49 (1H, br), 5.78 (1H, br d), 3.87 (2H, q), 3.71 (1H, m), 3.63 (3H, s), 3.07 (1H, t), 2.39 (1H, dd), 2.22 (2H, br d), 1.87-1.96 (1H, m), 1.47 (9H, s), 1.43 (9H, s), 1.05-1.08 (6H, m) and 0.95 (3H, t).

### Intermediate 52

### rel-(2S,4S,5R)- and rel-(2S,4R,5R)-2-Isobutyl-1-(3-methoxy-4-tert-butylbenzoyl)-4-hydrazino-carbonyl-5-(1,3-thiazol-2-yl)pyrrolidine-2-carboxylic acid, tert-butyl ester

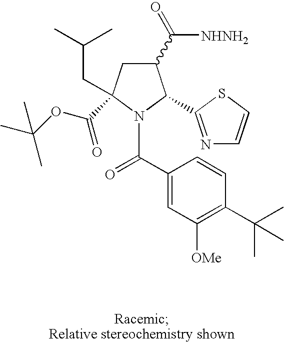

A mixture of rel-(2S,4S,5R)-2-Isobutyl-1-(3-methoxy-4-tert-butylbenzoyl)-5-(1,3-thiazol-2-yl)-pyrrolidine-2,4-dicarboxylic acid, 2-tert-butyl ester, 4-ethyl ester (Intermediate 51; 2.92 g, 5.09 mmol), hydrazine hydrate (10 mL) and ethanol (70 mL) was heated at reflux. Additional aliquots of hydrazine hydrate 5 mL) were added after both 6 hours and 9 hours and the reflux was continued for a total of 12 hours. The mixture was cooled and concentrated to dryness to afford the title compound, a solid, shown by NMR to be a mixture of the rel-(2S,4S,5R)- and rel-(2S,4R,5R)-diastereoisomers.

MS calcd for (C₂₉H₄₂N₄O₅S+H)⁺: 559. Found (M+H)⁺=559.

### Intermediate 53

### rel-(2S,4R,5R)-4-(5-Ethyl-1,2,4-oxadiazol-3-yl)-2-isobutyl -5-(1,3-thiazol-2-yl)pyrrolidine-2-carboxylic acid, tert-butyl ester

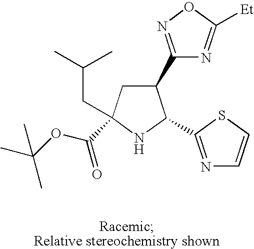

Stage A: Hydroxylamine hydrochloride (0.43 g, 6.14 mmol) was added to a solution of rel-(2S,4R,5R)-4-cyano-2-isobutyl-5-(1,3-thiazol-2-yl)pyrrolidine-2-carboxylic acid, tert-butyl ester (Intermediate 26; 1.03 g, 3.07 mmol) in ethanol (80 mL). Potassium hydroxide (0.34 g, 6.0 mmol) was added and the mixture heated at reflux for 11 hours. The mixture was allowed to cool to room temperature overnight, then concentrated.

Stage B: The residue was suspended in anhydrous dichloromethane (60 mL) containing triethyl,imine (0.59 mL, 4.27 mmol) and propanoic anhydride (0.81 mL, 6.31 mmol) was added. The resulting mixture was heated at 45° C. for 2 hours, then washed with water, saturated aqueous sodium bicarbonate solution and water again, then dried (Na₂SO₄).

Stage C: The resulting dichloromethane solution was treated with sodium hydride (60% dispersion in mineral oil; 0.30 g, 7.5 mmol) and heatyed at reflux. Additional aliquots of sodium hydride (60% dispersion; 0.5 g) were added after 2 hours and 20 hours total reaction time. After 21 hours total reaction time, the mixture was concentrated to ca. 10% of the original volume and heated at reflux for a further 18 hours, cooled and filtered. The solution was washed with water, dilute hydrochloric acid (2M) and water again, dried (Na₂SO₄) and evaporated. The residue was purified by chromatography on silica gel using cyclohexane-ethyl acetate (2:1 v/v) as eluent to afford the title compound, a gum.

MS calcd for (C₂₀H₃₀N₄O₃S+H)⁺: 407. Found: (M+H)⁺=407.

¹H NMR (CDCl₃): δ 7.69 (1H, d), 7.22 (1H, d), 4.92 (1H, d), 3.62 (1H, m), 2.90 (2H, q), 2.85 (1H, dd), 2.22 (1H, t), 1.70-1.84 (3H, m), 1.47 (9H, s), 1.39 (3H, t), 0.96 (3H, d) and 0.92 (3H, d). Pyrrolidine NH proton not identified.

### Intermediate 54

### rel-(2S,4R,5R)-4-(5-Cyclopropyl-1,2,4-oxadiazol-3-yl)-2-isobutyl-5-(1,3-thiazol-2-yl)pyrrolidine-2-carboxylic acid, tert-butyl ester

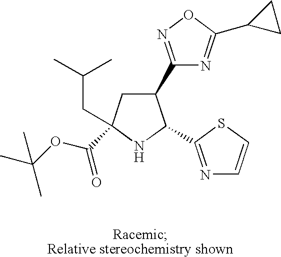

Stage A: Hydroxylamine hydrochloride (0.32 g, 4.66 mmol) was added to a solution of rel-(2S,4R,5R)-4-cyano-2-isobutyl-5-(1,3-thiazol-2-yl)pyrrolidine-2-carboxylic acid, tert-butly ester (Intermediate 26; 0.78 g, 2.33 mmol) in ethanol (60 mL). Potassium hydroxide (0.26 g, 4.55 mmol) was added and the mixture heated at reflux for 19 hours. The mixture was allowed to cool to room temperature overnight, then concentrated to afford the crude hydroxyamidine.

Stage B: The crude hydroxyamidine was suspended in anhydrous dichloromethane (40 mL) containing triethylamnine (0.32 mL, 2.31 mmol) and cyclopropanecarbonyl chloride (0.20 mL, 2.20 mmol) was added. The resulting mixture was stirred at room temperature for 18 hours then evaporated to dryness. The residue was suspended in dry THF (10 mL) and sodium hydride (60% dispersion in mineral oil; 0.060 g) was added. The mixture was stirred at room temperature for 20 hours then concentrated. The resulting material was partitioned between dilute hydrochloric acid (1M) and ethyl acetate. The ethyl acetate solution was washed with water, dried (Na₂SO₄) and evaporated. The crude product was then purified by chromatography on silica gel using cyclohexane-ethyl acetate (2:1 v/v) as eluent to afford the title compound, a gum.

MS calcd for (C₂₁H₂₃N₄O₃S+H)⁺: 419. Found: (M+H)⁺=419.

¹H NMR (CD₃OD): δ 7.69 (1H, d), 7.23 (1H, d), 4.90 (1H, d), 3.57 (1H, m), 2.83 (1H, dd), 2.15-2.21 (2H, m.), 1.69-1.84 (3H, m), 1.47 (9H, s), 1.17-1.24 (4H, m), 0.96 (3H, d) and 0.93 (3H, d). Pyrrolidine NH proton not identified.

### Intermediate 55

### 3-Bromo-5-methyl-1,2,4-thiadiazole

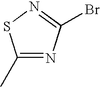

Oven dried zinc bromide (4.74 g, 21.0 mmol) was added to a cold (−78° C.) solution of methyl magnesium bromide (1.0 M solution in THF, 21.0 mL) in dry THF (84 mL) under nitrogen. The reaction mixture was stirred at −78° C. for 1 hour then allowed to warm to room temperature over a further 1 hour. Palladium tetrakis triphenylphosphine (0.42 g) and 3-bromo-5-chloro-1,2,4-thiadiazole (2.90 g, 14.30 mmol) were added and the mixture was then heated at 50° C. under nitrogen for 8 hours. The reaction mixture was cooled and evaporated to dryness. The residue was partitioned between ethyl acetate and saturated aqueous ammonium chloride. The ethyl acetate solution was washed with water, dried (Na₂SO₄) and evaporated to afford an oil which was subsequently) purified by chromatography on silica gel using cyclohexane-ethyl acetate (9:1 v/v) to afford the title compound as an oil.

¹H NMR (CDCl₃): δ 2.85 (3H, s).

### Intermediate 56

### 3-Vinyl-5-methyl-1,2,4-thiadiazole

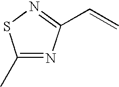

Oven dried zinc bromide (11.2 g) was added to a cold (−78° C.) solution of vinyl magnesium bromide (1.0 M solution in THF, 42.0 mL) in dry THF (160 mL) under nitrogen. The reaction mixture was stirred at −78° C. for 1 hour then allowed to warm to room temperature over a further 1 hour. Palladium tetrakis triphenylphosphine (0.84 g) and 3-bromo-5-methyl-1,2,4-thiadiazole (Intermediate 55; 1.25 g, 6.98 mmol) were added and the mixture was then heated at 50° C. under nitrogen for 48 hours. The reaction mixture was cooled to room temperature, filtered and evaporated. The residue was diluted with ethyl acetate and washed with saturated aqueous sodium bicarbonate solution and water, dried (Na₂SO₄) and evaporated to afford the title compound, an oil.

¹H NM (CDCl₃): δ 6.86 (1H, dd), 6.44 (1H, dd), 5.73 (1H, dd) and 2.80 (3H, s).

### Intermediate 57

### rel-(2S,4S,5R)-2-Isobutyl-4-(5-methyl-1,2,4-thiadiazol-3-yl)-5-(1,3-thiazol-2-yl)pyrrolidine-2-carboxylic acid, tert butyl ester

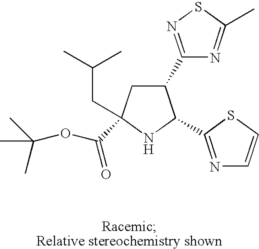

Lithium bromide (0.30 g, 3.47 mmol) was added to a solution of 3-vinyl-5-methyl-1,2,4-thiadiazole (Intermediate 56; 0.71 g, 5.63 mmol) and 2-[N-(1,3-thiazol-2-ylmethylene)amino]-4methyl-pentanoic acid, tert-butyl ester (Intermediate 1; 0.98 g, 3.47 mmol) in anhydrous THF (6 mL) under nitrogen. The mixture was stirred for 5 minutes at room temperature prior to the addition of triethylamine (1.44 mL, 10.41 mmol) and then stirred for an additional 3 days at room temperature before being diluted with saturated aqueous ammonium chloride solution and extracted with ethyl acetate. The ethyl acetate solution was washed with water, dried (Ma₂SO₄) and evaporated to afford a gum. This was purified by chromatography on silica gel using a gradient elution with cyclohexane-ethyl acetate (from 3:1 v/v to 2:1 v/v) to afford the title compound, an oil.

MS caled for (C₁₉H₂₈N₄O₂S₂+H)⁺: 409. Found: (M+H)⁺=409.

¹H NMR (CDCl₃): δ 7.49 (1H, d), 7.08 (1H, d), 5.08 (1H, d), 4.23 (1H, m), 2.99 (1H, dd), 2.64 (3H, s), 2.28 (1H, dd), 1.79-1.84 (2H, m), 1.67-1.72 (1H, m), 1.52 (9H, s), 0.99 (3H, d) and 0.94 (3H, d). Pyrrolidine NH proton exchanges with the solvent.

### Intermediate 58

### rel-(2S,4S,5R)-2-Methyl-4-(3-methyl-1,2,4-oxadiazol-5-yl)-5-(1,3-thiazol-2-yl)pyrrolidine-2-carboxylic acid, tert-butyl ester

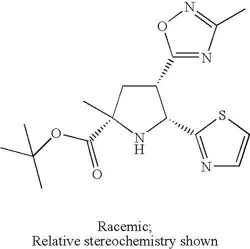

The title compound, an oil, was prepared from 2-aminopropanoic acid tert-butyl ester hydrochloride salt by sequentially following procedures analogous to those described for Intermediate 1, Intermediate 2 and Intermediate 24.

¹H NMR (CDCl₃): δ 7.72 (1H, d), 7.28 (1H, d), 5.06 (1H, d), 3.91 (1H, dd), 2.92 (1H, dd), 2.40 (3H, s), 2.26 (1H, dd), 1.52 (3H, s), 1.46 (9H, s). The pyrrolidine NH proton exchanges with the solvent.

### Intermediate 59

### 2-[Pyridin-2-ylmethylene)amino]-4-methylpentanoic acid, tert-butyl ester

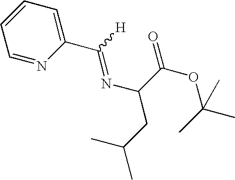

A stirred mixture of 2-amino4-methyl-pentanoic acid tert-butyl ester, hydrochloride salt (5.00 g, 22.34 mmol), pyridine-2-carboxaldehyde (2.12 mL, 22.34 mmol) and triethylamine (3.10 mL, 22.3 mmol) in dichloromethane (75 mL) were heated under reflux under nitrogen for 2 hours. The reaction mixture was allowed to cool to room temperature, washed with water and brine, dried over Na₂SO₄ and evaporated to give the title compound as an oil.

¹H NMR (CDCl₃): δ 8.65 (1H, ddd), 8.37 (1H, s), 8.12 (1H, dt), 7.75 (1H, ddt), 7.34 (1H, ddd), 4.05 (1H, dd), 1.79-1.85 (2H, m), 1.58 (1H, m), 1.47 (9H, s), 0.95 (3H, d) and 0.91 (3H, d).

### Intermediate 60

### rel-(2S,4S,5R)-4-(Aminocarbonyl)-2-isobutyl-5-pyridin-2-yl-pyrrolidine-2-carboxylic acid, tert-butyl ester

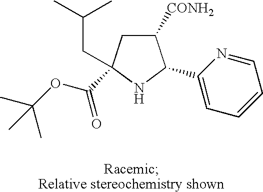

Acrylamide (2.36 g, 33.1 mmol) and lithium bromide (2.93 g, 33.1 mmol) were added to a solution of 2-[N-(pyridin-2-ylmethylene)amino]4-methylpentanoic acid, tert-butyl ester (Intermediate 59; 6.13 g, 22.18 mmol) in dry THF (40 mL) at 0° C. under nitrogen. The mixture was stirred at 0° C. for 5 hours then diluted with ethyl acetate (300 mL), washed with saturated aqueous ammonium chloride solution (200 mL), water (100 mL) and brine (100 mL) and dried over MgS0₄. The solvent was evaporated and the residue triturated with diethyl ether to afford the title compound, a solid.

¹HMR (CDCl₃): δ 8.54 (1H, d), 7.67 (1H, dt), 7.43 (1H, d), 7.18 (1H, dd), 6.06 (1H, br), 4.89 (1H, br), 4.65 (1H, d), 3.33 (1H, m), 2.66 (1H, dd), 2.14 (1H, dd), 1.88 (2H, m), 1.80 (1H, m), 1.65 (1H, dd), 1.52 (9H, s), 1.00 (3H, d) and 0.94 (3H, d).

### Intermediate 61

### Enantiomer A derived from rel-(2S,4S,5R)-4-(Aminocarbonyl)-2-isobutyl-5-pyridin-2-yl-pyrrolidine-2-carboxylic acid, tert-butyl ester

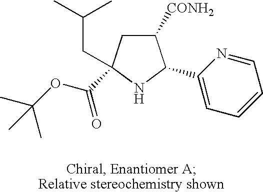

Stage A: A solution of (−)-di-O,O′-p-tolyl-L-tartaric acid (2.78 g, 7.19 mmol) in ethyl acetate (40 mL) was added to a solution of rel-(2S,4S,5R)-4-(aminocarbonyl)-2-isobutyl-5-pyridin-2-yl-pyrrolidine-2-carboxylic acid, tert-butyl ester (Intermediate 60; 2.50 g, 7.195 mmol) in ethyl acetate (250 mL). The resulting solution was stirred at room temperature overnight in a stoppered flask and the resulting crystalline solid filtered off to afford the tartrate salt of the title compound (3.11 g).

Stage B: A sample of the salt from Stage A (3.10 g) was partitioned between dichloromethane (100 mL) and saturated aqueous sodium bicarbonate solution (100 mL). The aqueous solution was extracted with dichloromethane (100 mL) and the combined dichloromethane solutions were washed with water (50 mL) and brine (50 mL.), dried (NgSO₄) and evaporated to afford the title compound, a solid. (0.98 g) Analytical HPLC using a Chiralpak AD chromatography column using heptane-ethanol (50:50 v/v) as eluent showed this solid to be the 1^{st} eluting enantiomer of the title compound (Enantiomer A; 94.3% ee; retention time 3.57 minutes), identical by ¹H NMR to the racemic compound described in Intermediate 60.

Chiral analytical HPLC of the corresponding racemate (Intermediate 60) shows two peaks with retention times 3.57 and 10.81 minutes for Enantiomers A and B respectively under identical chiral HPLC conditions.

### Intermediate 62

### Enantiomer A of rel-(2S,4S,5R)-4-(Aminocarbonyl)-1-(3-bromo-4-tert-butylbenzoyl)-2-isobutyl-5-pyridin-2-yl-pyrrolidine-2-carboxylic acid, tert-butyl ester

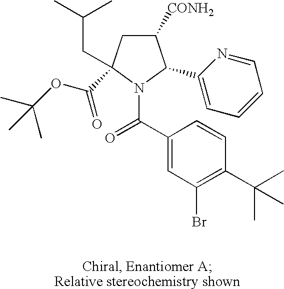

Enantiomer A derived from rel-(2S,4S,5R)-4-(aminocarbonyl)-2-isobutyl-5-pyridin-2-yl-pyrrolidine-2-carboxylic acid, tert-butyl ester (Intermediate 61) was acylated with 3-bromo-4-tert-butylbenzoyl chloride in a manner analogous to that described for Intermediate 3 to afford the title compound, a foam.

MS calcd for (C₃₀H₄₀BrN₃O₄+H)⁺: 586/588. Found: (M+H)⁺=586/588.

¹H NMR (CDCl₃): δ 8.06 (1H, d), 7.65 (1H, dt), 7.21 (1H, d), 7.08 (1H, dd), 6.88 (2H, m), 7.53 (1H, br s), 5.30 (1H, d), 5.05 (1H, br s), 3.67 (1H, m), 2.93 (1H, t), 2.31 (2H, m), 2.09 (1H, m), 1.95 (1H, m), 1.60 (9H, s), 1.41 (9H, s) and 1.09 (6H, d).

### Intermediate 63

### rel-(2S,4R,5R)-2-Isobutyl-4-(3-methyl-isoxazol-5-yl)-5-(1,3-thiazol-2-yl)pyrrolidine-2-carboxylic acid, tert-butyl ester

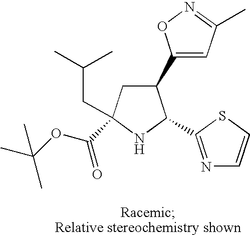

Stage A: A mixture of rel-(2S,4S,5R)- and rel-(2S,4R,5R)-4-acetyl-2-isobutyl-5-(1,3-thiazol-2-yl)pyrrol-idine-2-carboxylic acid, tert-butyl ester (Intermediate 9; 6.1 g, 17 mmol) and N,N-dimethylacetamide dimethyl acetal (25 mL) was heated at 110° C. under nitrogen for 11 hours, cooled and concentrated.

Stage B: The crude product from Stage A was dissolved in ethanol (155 mL) and hydroxylamine hydrochloride (3.57 g, 51.4 mmol) was added. The mixture was heated at reflux for 2 hours, then cooled and evaporated. The residue was dissolved in ethyl acetate (100 mL) and washed with water (100 mL) and brine (100 mL), dried (MgSO₄) and evaporated to afford an oil. This was partially purified by chromatography on silica gel using a gradient of cyclohexane-ethyl acetate (from 95:5 v/v to 85:15 v/v) as eluent to give the impure title compound. Repeat purification by additional chromatography on silica gel using a gradient of cyclohexane-ethyl acetate (from 95:5 v/v to 90:10 v/v) afforded the title compound, an oil. This material was shown by nOe NMR spectroscopy (of a subsequent derivative, Example 69) to be the rel-(2S,4R,5R)-diastereoisomer, inverted at the pyrrolidine C(4)-centre relative to the starting material.

MS calcd for (C₂₀H₂₉N₃O₃S+H)⁺: 392. Found: (M+H)⁺=392.

¹H NMR (CDCl₃): δ 7.71 (1H, d), 7.25 (1H, d), 5.94 (1H, s), 4.73 (1H, d), 3.53-3.60 (1H, m), 3.14 (1H, brs), 2.87 (1H, dd), 2.26 (3H, s), 2.14 (1H, t), 1.70-1.80 (3H, m), 1.49 (9H, s), 0.95 (3H, d) and 0.93 (3H, d).

### Intermediate 64

### rel-(2S,4S,5R)-4-(Aminocarbonyl)-2-isobutyl-5-(1,3-thiazol-4-yl)pyrrolidine-2-carboxylic acid, tert-butyl ester

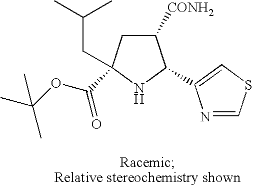

To a cooled (−5°), stirred solution of 2-[N-(1,3-thiazol4-ylmethylene)amino]-4methylpentanoic acid tert-butyl ester (Intermediate 48; 2.25 g, 7.98 mmol) in anhydrous THF (20 mL) under nitrogen, was added acrylamide (0.708 g, 9.97 mmol) followed by lithium bromide (1.39 g, 16 mmol). The resultant mixture was stirred at −5° for 5 minutes and then triethylamine (1.39 mL, 9.97 mmol) was added and stirring was continued at ambient temperature for 21 hours. Aqueous anmnonium chloride was added with rapid stirring and the resulting mixture was extracted twice with ethyl acetate. The extracts were combined and washed twice with water and once with brine. The organic solutions were evaporated and the residue triturated with diethyl ether to afford the title compound, a solid.

MS calcd for (C₁₇H₂₇N₃O₃S+H)⁺: 354. Found: (M+H)⁺=354

¹H NMR (CD₃OD): δ 8.90 (1H, s), 7.45 (1H, s), 4.65 (1H, d), 3.30 (1H, q), 2.65 (1H, dd), 2.05-2.10 (1H, dd), 1.80 (2H, m), 1.70 (1H, m), 1.50 (9H, s), 1.00 (3H, d) and 0.95 (3H, d). Pyrrolidine NH and amide protons exchange with solvent.

### Intermediate 65

### rel-(2S,4S,5R)-2-Isobutyl-4-(3-methyl-1,2,4-oxadiazol-5-yl)-5-(1,3-thiazol-4-yl)pyrrolidine-2-carboxylic acid, tert-butyl ester

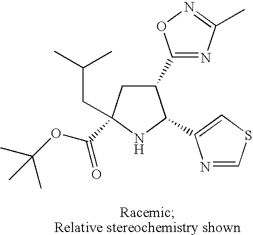

rel-(2S,4S,5R)-4-(Aminocarbonyl)-2-isobutyl-5-(1,3-thiazol-4-yl)pyrrolidine-2-carboxylic acid, tert-butyl ester (Intermediate 64; 1.57 g, 4.45 mmol) and (1,1-dimethoxyethyl)dimethylamine (20 mL) was heated at 120° C. for 2 hours then cooled and concentrated. The residue was dissolved in dioxan (8 mL) and acetic acid (8 mL) and hydroxylamine hydrochloride (0.433 g, 6.23 mmol) and aqueous sodium hydroxide (2M, 1.59 mL) were added. The mixture was heated at 90° C. for 3 hours, cooled and evaporated and the resulting material purified by chromatography on silica gel using cyclohexane-ethyl acetate (5:1 v/v) as eluent to afford the title compound, an oil.

MS calcd for (C₁₉F₂₁N₄O₃S+H)⁺: 393. Found: (M+H)⁺=393

¹H NMR (CD₃OD): δ 8.75 (1H, s), 7.40 (1H, s), 4.90 (1H, d), 4.00 (1H, q), 2.85-2.90 (1H, dd), 2.35-2.40 (1H, dd), 2.20 (3H, s), 1.75-1.90 (3H, m), 1.55 (9H, s), 1.00 (3H, d) and 0.95 (3H, d). Pyrrolidine NH proton exchanges with solvent.

### Intermediate 66

### rel-(2S,4R,5R)-2-Isobutyl-4-(3-methyl-1,2,4-oxadiazol-5-yl)-5-(1,3-thiazol-4-yl)pyrrolidine-2-carboxylic acid, tert-butyl ester

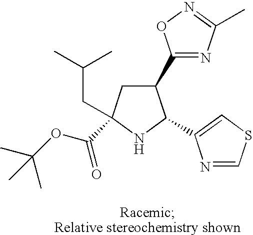

A mixture of rel-(2S,4S,5R)-2-Isobutyl-4-(3-methyl-1,2,4-oxadiazol-5-yl)-5-(1,3-thiazol-4-yl)pyrrolidine-2-carboxylic acid, tert-butyl ester (Intermediate 65; 0.70 g, 1.79 mmol) and methanolic sodium hydroxide (0.1M; 17.9 mL, 1.79 mmol; prepared from 2M aqueous sodium hydroxide diluted with methanol) was stirred at room temperature for 24 hours then concentrated. The residue was dissolved in dichloromethane, washed with dilute hydrochloric acid and brine, dried (Na₂SO₄) and evaporated to afford the title compound, an oil.

MS calcd for (C₁₉H₂₈N₄O₃S+H)⁺: 393. Found: (M+H)⁺=393.

¹H NMR (CD₃OD): δ 9.05 (1H, s), 7.75 (1H, s), 5.05 (1H, d), 4.05 (1H, q), 3.10 (1H, dd), 2.45-2.50 (1H, t), 2.35 (3H, s), 1.90-2.05 (2H, m), 1.80 (1H, m), 1.55 (9H, s) and 1.00 (6H, m). Pyrrolidine NH proton exchanges with solvent.

### Example 1

### rel-(2S,4R,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid

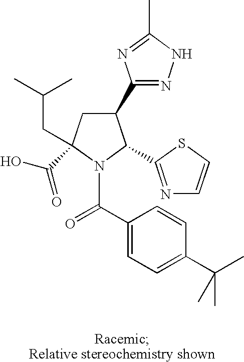

A mixture of rel-(2S,4S,5R)-4-(aminocarbonyl)-1-(4-tert-butylbenzoyl)-2-isobutyl-5-(1,3-thiazol-2-yl)pyrrolidine-2-carboxylic acid tert-butyl ester [Intermediate 3; 2.95 g, 6.45 mmol] and (1,1-dimethoxyethyl)dimethylamine (10 mL) were heated at 120° C. with stirring for 1.5 hours. The reaction mixture was evaporated to give an oil (4.26 g). A portion of this material (2.13 g) was treated with a solution of hydrazine monohydrate (0.31 mL, 6.40 mmol) in acetic acid (75 mL) and the resultant mixture was heated at 90° C. for 2.5 hours. The reaction mixture was allowed to cool to room temperature and was then evaporated. The residue was dissolved in trifluoroacetic acid (10 mL) and this solution was allowed to stand at room temperature for 3 days and was then evaporated. The residue was crystallised from ethyl acetate-ether (5:95 v/v) and the resulting solid was collected by filtration, washed successively with ether and ethyl acetate and finally dried in vacuo to give the title compound as a solid. This compound was shown by nuclear Overhauser enhancement (nOe) NMR experiments to be inverted at the pyrrolidine C(4) centre relative to the starting material.

MS calcd for (C₂₆H₃₃N₅O₃S+H)⁺: 496. Found: (M+H)⁺=496

¹H NMR (CD₃OD): δ 7.34 (d, 1H), 7.20 (AA′BB′, 4H), 7.12 (d, 1H), 5.80 (d, 1H), 3.98 (m, 1H), 2.82 (t, 1H), 2.62 (dd, 1H), 2.50 (br.m 1H), 2.40 (s, 3H), 2.10 (dd, 1H), 2.00 (m, 1H), 1.24 (s, 9H), 1.17 (d, 3H) and 1.00 (d, 3H). Triazole and carboxylic acid protons exchanged with solvent.

The corresponding rel-(2S,4S,5R)-diastereoisomer of the title compound was isolated from the crystallisation liquors and is described as Example 2 (below).

### Example 2

### rel-(2S,4S,5R)-1-(4-tert-butylbenzoyl)-2-isobutyl-4-(5-methyl-1H-1,2,4-triazol-3-yl)-5-(1,3-thiazol-2-yl)pyrrolidine2-carboxylic acid

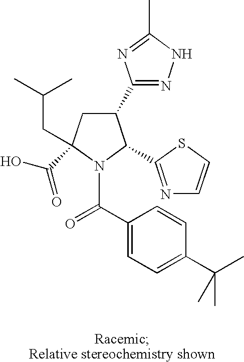

The crystallisation liquors from the crystallisation described in Example 1 (above) was washed with saturated aqueous sodium bicarbonate. A precipitate formed within the organic phase and this was collected by filtration to give the title compound, a solid.

MS calcd for (C₂₆H₃₃N₅O₃S+H)⁺: 496. Found: (M+H)⁺=496

¹H NMR (CD₃OD): δ 7.42 (br.s, 1H), 7.34 (d, 1H), 7.24 (½AA′BB′, 2H), 7.02 (½AA′BB′, 2H), 5.80 (d, 1H), 4.30 (m, 1H), 3.10 (t, 1H), 2.55-2.40 (m, 2), 2.30 (m, 1H), 2.19 (s, 3H), 2.08 (m, 1H), 1.22 (s, 9H), 1.18 (d, 3H) and 1.10 (d,3H). Triazole and carboxylic acid protons exchanged with the solvent.

### Example 3

### rel-(2S,4S,5R)-1-(4-tert-butylbenzoyl)-2-isobutyl-4-(3-methyl-1,2,4oxadiazol-5-yl)-5-(1,3-thiazol-2-yl)pyrrolidine-2-carboxylic acid

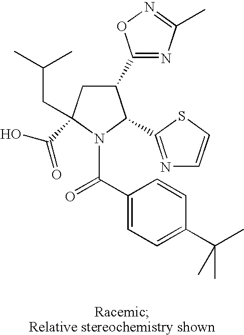

A mixture of rel-(2S,4S,5R)-4-(aminocarbonyl)-1-(4-tert-butylbenzoyl)-2-isobutyl-5-(1,3-thiazol-2-yl)pyrrolidine-2-carboxylic acid tert-butyl ester (Intermediate 3; 2.95 g, 6.45 mmol) and (1,1-dimethoxyethyl)dimethylamine (10 mL) were heated at 120° C. with stirring for 1.5 hours. The reaction mixture was evaporated to give an oil (4.26 g). Part of this material (2.13 g) was dissolved in a mixture of dioxan (5 mL) and acetic acid (5 mL) and to the resultant solution was added hydroxylamine hydrochloride (0.31 g, 4.51 mmol), followed by aqueous sodium hydroxide (2 M, 3.3 mL). This mixture was heated at 90° C. for 1.75 hours. The reaction mixture was allowed to cool to room temperature and was then evaporated. The residue was dissolved in trifluoroacetic acid (10 mL) and this solution was allowed to stand at room temperature for 3 days and was then evaporated. The residue was partially separated by chromatography on silica gel using ethyl acetate-cyclohexane (1:1 v/v) as eluent. The early fractions were combined and then evaporated and the residue was further purified by reverse phase HPLC on a C₁₈ column using a two-solvent gradient elution with (A) water containing formic acid (0.1%) and (B) acetonitrile-water (95:5 v/v) containing formic acid (0.05%) as the eluents. The title compound was isolated as a solid.

MS calcd for (C₂₆H₃₂N₄O₄S+H)⁺: 496. Found: (M+H)⁺=496.

¹H NMR (CDCl₃): δ 7.76 (d, 1H), 7.22 (½AA′BB′, 2H), 7.17 (d, 1H), 6.95 (½AA′BB′, 2H), 5.77 (d, 1H), 4.38 (m, 1H), 3.18 (t, 1H), 2.62 (dd, 1M), 2.48-2.35 (2×dd, 2H), 2.20 (s, 3H), 1.98 (m, 1H), 1.24 (s, 9H), 1.18 (d, 3H) and 1.14 (s, 3H). The carboxylic acid signal was not seen.

Continued elution of the silica gel chromatography column afforded the rel-(2S,4R,5R)-diastereoisomer, described further as Example 4, (below).

### Example 4

### rel-(2S,4R,5R)-1-(4-tert-butylbenzoyl)-2-isobutyl4-(3-methyl-1,2,4-oxadiazol-5-yl)-5-(1,3-thiazol-2-yl)pyrrolidine2-carboxylic acid

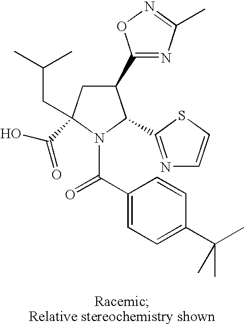

The rel-(2S,4R,5R)-diastereoisomer isolated from the silica gel chromatography column described in Example 3 (above) was further purified by reverse phase HPLC on a C₁₈ column using a two-solvent gradient elution with (A) water containing formic acid (0.1%) and (B) acetonitrile-water (95:5 v/v) containing formic acid (0.05%) as the eluents. The title compound was isolated as a solid.

MS calcd for (C₂₆H₃₂N₄O₄S+H)⁺: 496. Found: (M+H)⁺=496.

¹H NMR (CD₃OD): δ 7.39 (d, 1H), 7.22 (½AA′BB′, 2H), 7.20-7.16 (½AA′BB′, 3H), 5.82 (d, 1H), 4.20 (m, 1H), 2.88 (t, 1H), 2.76 (dd, 1H), 2.50 (br.m, 1H), 2.35 (s, 3H), 2.05 (dd, 1H), 1.95 (m, 1H), 1.24 (s, 9H), 1.16 (d, 3H) and 0.98 (d, 3H). The carboxylic acid signal was exchanged with the solvent.

### Example 5

### rel-(2S,4S,5R)-1-(4-tert-butylbenzoyl)-2-isobutyl-5-(1,3-thiazol-2-yl)-4-(1H-1,2,4-triazol-3-yl)pyrrolidine-2-carboxylic acid

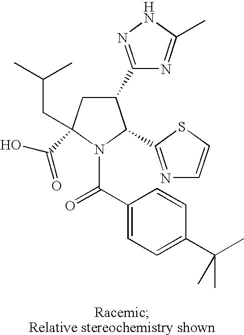

A mixture of rel-(2S,4S,5R)-4-(aminocarbonyl)-1-(4-tert-butylbenzoyl)-2-isobutyl-5-(1,3-thiazol-2-yl)-pyrrolidine-2-carboxylic acid tert-butyl ester (Intermediate 3; 1.13 g, 2.46 mmol) and (1,1-dimethoxymethyl)dimethylamine (15 mL) were heated at 120° C. with stirring for 1.5 hours. The reaction was evaporated to give an oil (1.53 g). Part of this material (0.76 g) was dissolved in acetic acid (30 mL), hydrazine monohydrate (0.31 mL, 6.40 mmol) was added and the resultant mixture was heated at 90° C. for 2.5 hours. The reaction was allowed to cool to room temperature and was then evaporated. The residue was dissolved in trifluoroacetic acid (10 mL) and this solution was stirred at room temperature for 19 hours and was then evaporated. The residue was partially purified by chromatography on silica gel using firstly ethyl acetate-cyclohexane (3:1 v/v) followed by ethyl acetate-cyclohexane (9:1 v/v) as eluent. The fractions containing the desired product were combined and evaporated. The residue was further purified by crystallisation from ethyl acetate to give the title compound as a solid.

MS calcd for (C₂₅H₃₁N₅O₃S+H)⁺: 482. Found: (M+H)⁺=482.

¹H NMR (CD₃OD): δ 8.20 (v.br.s, 1H), 7.72 (d, 1H), 7.36 (d, 1H), 7.32 (½AA′BB′, 2H), 7.07 (½AA′BB′, 2H), 5.96 (d, 1H), 4.48 (m, 1H), 3.14 (t, 1H), 2.60 (dd, 1H), 2.43-2.28 (m, 2H), 2.15 (m, 1H), 1.27 (s, 9H), 1.20 (d, 3H) and 1.16 (d, 3H). Triazole and carboxylic acid protons exchanged with solvent.

### Example 6

### rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid

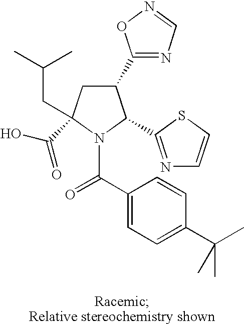

A mixture of rel-(2S,4S,5R)-4-(aminocarbonyl)-1-(4-tert-butylbenzoyl)-2-isobutyl-5-(1,3-thiazol-2-yl)-pyrrolidine-2-carboxylic acid tert-butyl ester (Intermediate 3; 0.83 g,1.61 mmol) and (1,1-dimethoxymethyl)dimethylamine (15 mL) were heated at 120° C. with stirring for 1.5 hours. The residue was dissolved in a mixture of dioxan (6 mL) and acetic acid (6 mL) and to the resultant solution was added hydroxylamine hydrochloride (0.16 g, 2.25 mmol) followed by aqueous sodium hydroxide (2 M, 1.25 mL). This mixture was heated at 90° C. for 2.5 hours. The reaction mixture was allowed to cool to room temperature and was then evaporated. The residue was dissolved in trifluoroacetic acid (8 mL) and this solution was stirred at room temperature for 3 hours and was then evaporated. The residue was partitioned between ethyl acetate and saturated aqueous sodium bicarbonate and the organic phase was separated and evaporated. The residue was purified successively by reverse phase HPLC on a C₁₈ column using a two-solvent gradient elution with (A) water containing formic acid (0.1%) and (B) acetonitrile-water (95:5 v/v) containing formic acid (0.05%) as the eluets, followed by crystallisation from ethyl acetate-ether-cyclohexane to give the title compound as a solid.

MS calcd for (C₂₅H₃₀N₄O₄S+H)⁺: 483. Found: (M+H)⁺=483.

¹H NMR (CD₃OD): δ 8.40 (s, 1H), 7.67 (d, 1H), 7.42 (d, 1H), 7.32 (½AA′BB′, 2H), 7.10 (½AA′BB′, 2H), 6.18 (d, 1H), 4.73 (m, 1H), 3.15 (t, 1H), 2.74 (dd, 1H), 2.40-2.28 (m, 2H), 2.14 (m, 1H), 1.28 (s, 9H), 1.18 (d, 3H) and 1.15 (d, 3H). Carboxylic acid proton exchanged with solvent.

### Example 7

### rel-(2S,4R,5R)-1-(4tert-butylbenzoyl)-2-isobutyl-4-(1,2,4-oxadiazol-5-yl)-5-(1,3-thiazol-2-yl)pyrrolidine-2-carboxylic acid

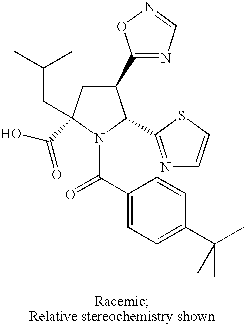

The title compound was prepared by analogy with the method described for Example 6, with the exception that the ratio of sodium hydroxide to starting material used in the reaction was increased from 1.55:1 molar equivalents to 5.36:1 molar equivalents. This compound was shown by nOe NMR experiments to be inverted at the pyrrolidine C(4) centre relative to the starting material.

MS calcd for (C₂₅H₃₀N₄O₄S+H)⁺: 483. Found: (M+H)⁺=483.

¹H NMR (CD₃OD): δ 8.62 (s, 1H), 7.40 (d, 1H), 7.35-7.18 (½AA′BB′, 5H), 5.84 (d, 1H), 4.30 (m, 1H), 2.92 (dd, 1H), 2.80 (dd, 1H), 2.50 (br.d, 1H), 2.10 (dd, 1H), 1.96 (m, 1H), 1.24 (s, 9H), 1.16 (d, 3H) and 1.00 (d, 3H). Carboxylic acid proton exchanged with solvent.

### Example 8

### rel-(2S,4R,5R)-1-(4-tert-butylbenzoyl)-2-isobutyl-4-(5-methyl-1H-1,2,4-triazol-3-yl)-5-thein-2-yl-pyrrolidine-2-carboxylic acid

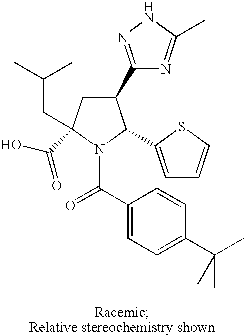

The title compound was prepared according to the method described in Example 1 substituting rel-(2S,4S,5R)-4-(aminocarbonyl)-1-(4-tert-butylbenzoyl)-2-isobutyl-5-thien-2-yl-pyrrolidine-2-carboxylic acid tert-butyl ester (Intermediate 6) in place of rel-(2S,4S,5R)-4-(Aminocarbonyl)-1-(4-tert-butylbenzoyl)-2-isobutyl-5-(1,3-thiazol-2-yl)-pyrrolidine-2-carboxylic acid tert-butyl ester, and was shown by nOe NMR experiments to be inverted at the pyrrolidine C(4) centre relative to the starting material.

MS calcd for (C₂₇H₃₄N₄O₃S+H)⁺: 495. Found: (M+H)⁺=495.

¹H NMR (CD₃OD). δ 7.22 (AA′BB′, 4H), 7.02 (br.d, 1H), 6.42 (dd, 1H), 6.35 (br.d, 1H), 5.68 (d, 1H), 3.93 (m, 1H), 2.67 (t, 1H), 2.58-2.52 (2×m, 2H), 2.40 (s, 3H), 2.12-1.98 (m, 2H), 1.28 (s, 9H), 1.18 (d, 3H) and 1.04 (d, 3H). Triazole and carboxylic acid protons exchanged with solvent.

### Example 9

### 2^{nd} Eluting Enantiomer derived from rel-(2S,4R,5R)-1-(4tert-butylbenzoyl)-2-isobutyl-4-(5-methyl-1H-1,2,4-triazol-3-yl)-5-(1,3-thiazol-2-yl)pyrrolidine-2-carboxylic acid

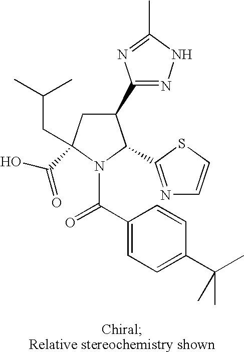

rel-(2S,4R,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid (Example 1) was resolved by preparative HFLC on a Chiralpak AD chromatography column using heptane-ethanol (95:5 v/v) containing 0.1% trifluoroacetic acid as eluent to afford the individual enantiomers with retention times of 7 minutes (1^{st} Eluting Enantiomer) and 9 minutes (2^{nd} Eluting Enantiomer the title compound) respectively. The 2^{nd} Eluting Enantiomer was identical by ¹H NMR to the racemic compound described for Example 1.

### Example 10

### 2^{nd} Eluting Enantiomer derived from rel-(2S,4s,5R)-1-(4-tert-butylbenzoyl)-2-isobutyl-5-(1,3-thiazol-2-yl)-4-(1H-1,2,4-triazol-3-yl)pyrrolidine-2-carboxylic acid

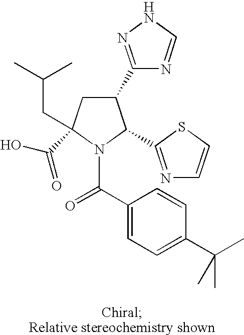

rel-(2S,4S,5R)-1-(4-tert-butylbenzoyl)-2-isobutyl-5-(1,3-thiazol-2-yl)-4-(1H-1,2,4-triazol-3-yl)pyrrolidine-2-carboxylic acid (Example 5) was resolved by preparative HPLC on a Chiralpak AD chromatography column using heptane-ethanol (95:5 v/v) containing 0.1% trifluoroacetic acid as eluent to afford the individual enantiomers with retention times of 20 minutes (1^{st} Eluting Enantiomer) and 27 minutes (2^{nd} Eluting Enantiomer; the title compound) respectively. The 2^{nd} Eluting Enantiomer was identical by ¹H NMR to the racemic compound described for Example 5.

### Example 11

### 2^{nd} Eluting Enantiomer derived from rel-(2S,4R,5R)-1-(4-tert-butylbenzoyl)-2-isobutyl-4-(3-methyl-1,2,4-oxadiazol-5-yl)-5-(1,3-thiazol-2-yl)pyrrolidine-2-carboxylic acid

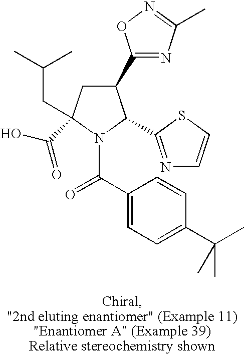

rel-(2S,4R,5R)-1-(4-tert-butylbenzoyl)-2-isobutyl-4-(3-methyl-1,2,4-oxadiazol-5-yl)-5-(1,3-thiazol-2-yl)pyrrolidine-2-carboxylic acid (Example 4) was resolved by preparative HPLC on a Chiralpak AD chromatography column using heptane-ethanol (95:5 v/v) containing 0.1% trifluoroacetic acid as eluent to afford the individual enantiomers with retention times of 8 minutes (1^{st} Eluting Enantiomer) and 13 minutes (2^{nd} Eluting Enantiomer: the title compound) respectively. The 2^{nd} Eluting Enantiomer was identical by ¹H NMR to the racemic compound described for Example 4, and corresponds to Enantiomer A described in Example 39.

### Example 12

### rel-(2S,4R,5R)-1-(4-tert-butylbenzoyl)-2-isobutyl-4-(3-methyl-1,2,4-oxadiazol-5-yl)-5-thien-2-ylpyrrolidine-2-carboxylic acid

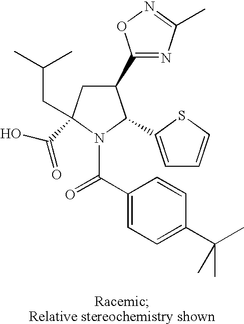

The title compound was prepared and isolated by analogy with the methods described in Examples 3 and 4, substituting rel-(2S,4S,5R)-4-(aminocarbonyl)-1-(4-tert-butylbenzoyl)-2-isobutyl-5-thien-2-ylpyrrolidine-2-carboxylic acid tert-butyl ester (Intermediate 6) in place of rel-(2S,4S,5R)-4-(aminocarbonyl)-1-(4-tert-butylbenzoyl)-2-isobutyl-5-(1,3-thiazol-2-yl)pyrrolidine-2-carboxylic acid tert-butyl ester, and was shown by nOe NMR experiments to be inverted at the pyrrolidine C(4) centre relative to the starting material.

MS calcd for (C₂₇H₃₃N₃O₄S+H)⁺: 496. Found: (M+H)⁺=496.

¹H NMR (CD₃OD): δ 7.22 (½AA′BB′, 2H), 7.16 (½AA′ BB′, 2H), 7.06 (t, 1H), 6.42 (d, 2H), 5.64 (d, 1H), 4.16 (m, 1H), 2.78-2.62 (m, 2H), 2.57 (br.m, 1H), 2.32 (s, 3H), 2.02 (m, 1H), 1.95 (m, 1H), 1.24 (s, 9H), 1.18 (d, 3H) and 1.00 (d, 3H). The carboxylic acid signal was exchanged with the solvent.

### Example 13

### rel-(2S,4S,5R)-1-(4-tert-butylbenzoyl-2-isobutyl-4-(5-methyl-1,2,4-oxadiazol-3-yl)-5-(1,3-thiazol-2-yl)pyrrolidine-2-carboxylic acid

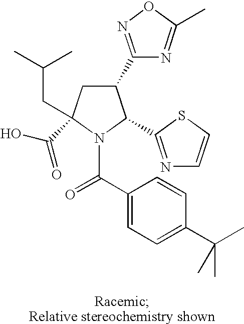

A mixture of rel-(2S,4S,5R)- and rel-(2S,4R,5R)-4-cyano-2-isobutyl-1-(4-tert-butylbenzoyl)-5-(1,3-thiazol-2-yl)pyrrolidine-2-carboxylic acid, tert-butyl ester (Intermediate 8; 3.77 g, 7.61 mmol), hydroxylamine hydrochloride (0.86 g 12.30 mmole) and potassium hydroxide (0.59 g, 10.56 mmole) in ethanol (120 mL) were heated at reflux for 4.5 hours. This mixture was evaporated to dryness and a portion of the resulting crude hydroxyamidine (2.00 g) was treated with N,N-dimethylacetamide dimethylacetal (20 mL) and heated at 100° C. with stirring under nitrogen for 1.5 hours. The reaction mixture was allowed to cool to room temperature and was then evaporated to dryness to give a brown semi-solid. This material was partially dissolved in trifluoroacetic acid (8 mL) and the resultant suspension was stirred at room temperature for 3.5 hours and was then evaporated to give a brown oil. Water (100 mL) was added with rapid stirring and after 10 minutes the stirring was stopped and the mixture was allowed to stand for 15 minutes. The supernatant liquid was carefully decanted off to leave a brown gum which was then crystallised from acetonitrile to give the title compound as a white solid.

MS calcd for (C₂₆H₃₂N₄O₄S+H)⁺: 497. Found: (M+H)⁺=497.

¹H NMR (DMSO-d₆): δ 7.63 (d, 1H), 7.52 (d, 1H), 7.25 (½AA′BB′, 2H), 7.06 (½AA′BB′, 2H), 5.98 (d, 1H), 4.38 (m, 1H), 2.80 (t, 1H), 2.58-2.51 (m, 1H), 2.38 (s, 3H), 2.24-2.12 (m, 2H), 2.02 (m, 1H), 1.21 (s,9H), 1.03 (d, 3H) and 1.05 (d, 3H).

### Example 14

### rel-(2S,4S,5R)-1-(4-tert-butylbenzoyl)-2-isobutyl-4-(1,2,4-oxadiazol-3-yl)-5-(1,3-thiazol-2-yl)pyrrolidine-2-carboxylic acid

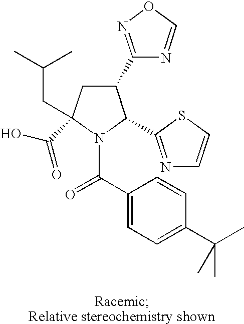

In an analogous manner to that described in Example 13, rel-(2S,4S,5R)- and rel-(2S,4R,5R)-4-cyano-2-isobutyl-1-(4-tert-butylbenzoyl)-5-(1,3-thiazol-2-yl)pyrrolidine-2-carboxylic acid, tert-butyl ester (Intermediate 8) was treated with hydroxylamine and potassium hydroxide in ethanol at reflux. A portion of the resulting crude hydroxyamidine (0.66 g) was treated with acetic-formic anhydride (21 mL) and stirred at room temperature for 4 hours, heated at 100° C. for 10 minutes and then evaporated. The residue was dissolved in toluene (50 mL), heated at reflux for 4.5 hours and then evaporated to dryness leaving an orange-brown gum (0.76 g). This material was chromatographed on silica gel using initially ethyl acetate-cyclohexane (1:1 v/v) and then ethyl acetate as eluent to afford a foam. This foam was dissolved in trifluoroacetic acid (6 mL) and the solution was stirred at room temperature for 4 hours and then evaporated to dryness. The residue was purified successively by reverse phase HPLC on a C₁₈ column using a two-solvent gradient elution with (A) water containing formic acid (0.1%) and (B) acetonitrile-water (95:5 v/v) containing forric acid (0.05%) as the eluents, followed by preparative silica gel TLC plate chromatography using ethyl acetate as eluent to afford the title compound, a solid.

MS calcd for (C₂₅H₃₀N₄O₄S+H)⁺: 483. Found (M+H)⁺=483.

¹H NMR (CD₃OD): δ 8.99 (s, 1H), 7.66 (d, 1H), 7.38 (d, 1H), 7.29 (½AA′BB′, 2H), 7.06 (½AA′BB′, 2H), 6.04 (d, 1H), 4.59-4.50 (m, 1H), 3.07 (t, 1H), 2.625 (dd, 1H), 2.38-2.25 (m, 2H), 2.16-2.05 (m, 1H), 1.24 (s, 9H), 1.165 (d, 31) and 1.125 (d, 3H).

### Example 15

### rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxamide

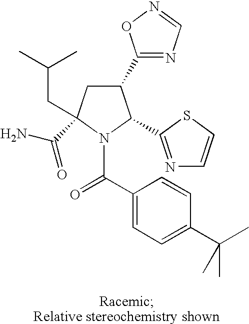

A mixture of rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid (Example 6; 0.11 g, 0.22 mmol), diisopropylethylamine (0.073 mL, 0.42 mmol), [O-(7-azabenzotriazol-1-yl)-1,1,3,3-tetramethyluronium]hexafluoro-phosphate (HATU) (0.087 g,0.23 mmol), ammonium chloride (0.22 g, 0.42 mmol) and anhydrous N,N-dimethylformamide (10 mL) was stirred at room temperature for 26 hours. Additional portions of HATU (0.087 g, 0.23 mmol), ammonium chloride (0.22 g, 0.42 mmol) and diisopropylethylamine (0.073 mL, 0.42 mmol) were added and the mixture was stirred for a further 18 hours. The reaction mixture was evaporated to dryness and the residue was purified by reverse phase HPLC on a C₁₈ column using a two-solvent gradient elution with (A) water containing formic acid (0.1%) and (B) acetonitrile-water (95:5 v/v) containing formic acid (0.05%) as the eluents, to give the title compound as a solid.

MS calcd for (C₂₅H₃N₅O₃S+H)⁺: 482. Found (M+H)⁺=482.

¹H NMR (DMSO-d₆): δ 8.94 (bs, 1H), 8.71 (s, 1H), 7.68 (d, 1H), 7.46 (m, 1H), 7.21 (½AA′BB′, 2H), 7.12 (bs, 1H), 6.94 (½AA′BB′, 2H), 6.01 (d, 1H), 4.58 (m, 1H), 3.00 (t, 1H), 2.61 (dd, 1H), 2.22 (dd, 1H), 2.14-2.05 (m, 1H), 2.05-1.96 (m, 1H), 1.20 (t, 9H), 1.038 (d, 3H) and 1.034 (d, 3H).

### Example 16

### rel-(2S,4S,5R)-1-(3-Bromo-4-tert-butylbenzoyl)-2-isobutyl-4-(1,2,4-oxadiazol-5-yl)-5-(1,3-thiazol-2-yl)pyrrolidine-2-carboxylic acid

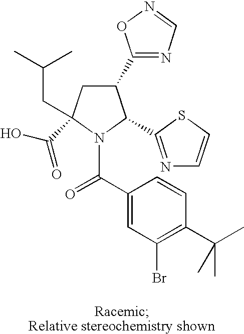

rel-(2S,4S,5R)-4-(Aminocarbonyl)-2-isobutyl-5-(1,3-thiazol-2-yl)pyrrolidine-2-carboxylic acid, tert-butyl ester was acylated with 3-bromo4-tert-butylbenzoyl chloride in a similar manner to that descrided for Intermediate 3 and the resulting rel-(2S,4S,5R)-4-(aminocarbonyl)-1-(3-bromo4-tert-butylbenzoyl)-2-isobutyl-5-(1,3-thiazol-2-yl)-pyrrolidine-2-carboxylic acid tert-butyl ester was then converted into the corresponding 1,2,4-oxadiazole title compound in a similar manner to that described in Example 6.

MS calcd for (C₂₅H₂₉BrN₄O₄S+H)⁺: 561/563. Found (M+H)⁺=561/563.

¹H NMR (DMSO-d₆): δ 8.71 (s, 1H), 7.555 (d, 1H), 7.495 (d, 1H), 7.34 (d, 1H), 7.25 (bs, 1H), 7.12 (d, 1H), 6.09 (d, 1H), 4.71-4.58 (m, 1H), 2.93 (t, 1N), 2.665 (dd, 1H), 2.21 (dd, 2H), 2.05-1.95 (m, 1H), 1.89 (s, 9H) and 1.03 (d, 6H). Carboxylic acid proton exchanged with solvent.

### Example 17

### rel-(2S,4R,5R)-1-(4-tert-butylbenzoyl)-2-isobutyl-4-[5-(4H)-1,2,4-oxadiazolon-3-yl)-5-(1,3-thiazol-2-yl)pyrrolidine-2-carboxylic acid

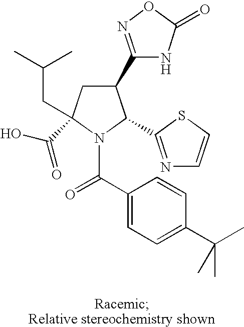

rel-(2S,4S,5R)-4-Cyano-2-isobutyl-1-(4-tert-butylbenzoyl)-5-(1,3-thiazol-2-yl)-pyrrolidine-2-carboxylic acid, tert-butyl ester (Intermediate 8; 0.42 g, 0.85 mmol) was added to a solution of hydroxylamine hydrochloride (0.095 g, 1.37 mmol) and potassium hydroxide (0.067 g, 1.20 mmol) in ethanol (65 mL) and the resultant mixture was heated at reflux for 6 hours. The reaction was allowed to cool to room temperature overnight and was then evaporated to dryness to give a solid. This solid was dissolved in dioxan (10 mL) containing dry pyridine (1 mL). Ethyl chloroformate (0.14 mL, 1.50 mmol) was added and the reaction mixture was heated at reflux for 3 hours then evaporated. The residue was partially purified by chromatography on silica gel using successively dichloromethane, then chloroform, then cyclohexane-ethyl acetate (4:1 v/v), then diethyl ether and finally ethyl acetate as eluents and then further purified by reverse phase HPLC on a C₁₈ column using a two-solvent gradient elution with (A) water containing formic acid (0.1%) and (13) acetonitrile-water (95:5 v/v) containing formic acid (0.05%) as the eluents. The resulting title compound tert-butyl ester (62 mg) was dissolved in trifluoroacetic acid (3 mL) and the solution allowed to stand at room temperature overnight and then evaporated to dryness. The residue was co-evaporated from dichloromethane and then twice from toluene before being suspended in diethyl ether and stirred for 2 h. The resulting solid was filtered off and dried in vacuo to afford the title compound. This was shown by nOe NMR experiments to be the rel-(2S,4R,5R)-diastereoisomer.

MS calcd for (C₂₅H₃₀N₄O₅S+H)⁺: 499. Found (M+H)⁺=499.

¹H NMR (CD₃OD): δ 7.41 (1H, d), 7.31-7.20 (5H, m), 5.78 (1H, d), 3.85 (1H, m), 2.71 (2H, m), 2.50 (1H, br), 2.11 (1H, m), 1.96 (1H, m), 1.28 (9H, m), 1.16 (3H, d) and 1.02 (3H, d). The CO₂H and NH protons exchanged with solvent.

### Example 18

### rel-(2S,4R,5R)-1-(4-tert-butylbenzoyl)-2-isobutyl-4-(isoxazol-5-yl)-5-(1,3-thiazol-2-yl)pyrrolidine-2-carboxylic acid

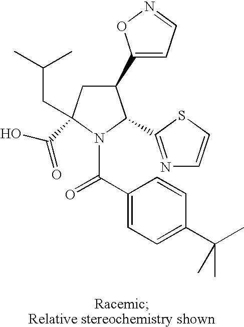

A mixture of rel-(2S,4R,5R)-4-acetyl-2-isobutyl-1-(4-tert-butylbenzoyl)-5-(1,3-thiazol-2-yl)-pyrrolidine-2-carboxylic acid, tert-butyl ester (Intermediate 10; 0.35 g, 0.68 mmol) and N,N-dimethylformamide dimethylacetal (5 mL) were heated at 110° C. under nitrogen for 5 hours. The reaction mixture was allowed to cool to room temperature and evaporated to give a crude intermediate 4-[3-(dimethylamino)prop-2-enoyl]-analogue as a gum. This material was mixed with ethanol (10 mL) and hydroxylamine hydrochloride (0.17 g, 2.45 mmol) and heated at reflex for 2 hours and then evaporated. The residue was suspended in trifluoroacetic acid (10 mL), stirred at room temperature for 4 hours then evaporated. The resulting oil was partially purified using reverse phase HPLC on a C₁₈ column using a two-solvent gradient elution with (A) water containing formic acid (0.1%) and (B) acetonitrile-water (95:5 v/v) containing fonnic acid (0.05%) as the eluents. The product was crystallised from ethyl acetate to give the title compound as a solid.

MS calcd for (C₂₆H₃₁N₃O₄S+H)⁺: 482. Found (M+H)⁺=482.

¹H NMR (CD₃OD): δ 8.265 (d, 1H), 7.355 (dd, 1H), 7.24 (½AA′BB′, 2H), 7.19 (½AA′BB′, 2H), 7.145 (d, 1H), 6.225 (d, 1H), 5.66 (d, 1H), 4.20-4.06 (m, 1H), 2.77 (t, 1H), 2.68 (dd, 1H), 2.57 (br d, 1H), 2.07 (dd, 1H), 1.99-1.91 (m, 1H), 1.25 (s, 9H), 1.17 (d, 3H) and 1.01 (d, 3H). The carboxylic acid proton was exchanged with solvent.

### Example 19

### rel-(2S,4R,5R)-1-(4-tert-butylbenzoyl)-2-isobutyl-4-(3-methylisoxazol-5-yl)-5-(1,3-thiazol-2-yl)pyrrolidine-2-carboxylic acid

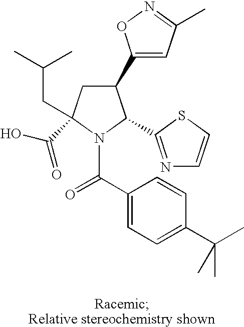

The title compound, a solid, was prepared in a similar manner to the method described for Example 18, substituting N,N-dimethylacetamide dimethyl acetal in place of N,N-dimethylformamide dimethyl acetal.

MS calcd for (C₂₇H₃₃N₃O₄S+H)⁺: 496. Found: (M+H)⁺=496.

¹H NMR (CD₃OD): δ 7.37 (1H, d), 7.24 (2H, d), 7.18 (2H, d), 7.13 (1H, br d), 6.10 (1H, s), 6.54 (1H, d), 4.08 (1H, m), 2.74 (1H, t), 2.65-2.53 (1H, br s), 2.64 (1H, dd), 2.19 (3H, s), 2.04 (1H, m), 1.94 (1H, m), 1.25 (9H, s), 1.17 (3H, s) and 1.01 (3H, s). Carboxylic acid proton exchanged with solvent.

### Example 20

### rel-(2S,4R,5R)-1-(4-tert-butylbenzoyl)-2-isobutyl-4-(3-methylpyrazol-5-yl)-5-(1,3-thiazol-2-yl)pyrrolidine-2-carboxylic acid

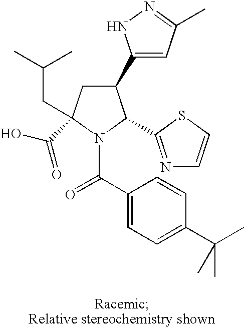

The title compound, a solid, was prepared in a similar manner to the method described for Example 19, substituting hydrazine hydrate in place of hydroxylamine.

MS calcd for (C₂₇H₃₄N₄O₃S+H)⁺: 495. Found: (M+H)⁺=495.

¹H NMR (DMSO-d₆): δ 12.65 (2H, br s), 7.39 (1H, d), 7.19 (2H, d), 7.12 (2H, d), 7.09 (1H, br s), 5.84 (1H, br s), 5.54 (1H, d), 3.73 (1H, m), 2.56-2.36 (3H, m), 2.14 (3H, s), 1.95-1.84 (2H, m), 1.22 (9H, s), 1.09 (3H, d) and 0.93 (3H, d).

### Example 21

### rel-(2S,4R,5R)-1-(4-tert-butylbenzoyl)-2-isobutyl-4-(1,3-thiazol-2-yl)-5-(1,3-thiazol-2-yl)pyrrolidine-2-carboxylic acid

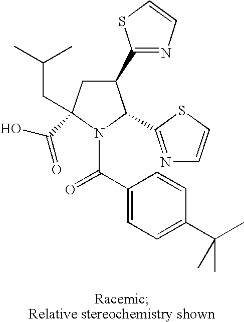

A mixture of rel-(2S,4S,5R)-2-Isobutyl-1-(4-tert-butylbenzoyl)-5-(1,3-thiazol-2-yl)-4-thiocarb-amoylpyrrolidine-2-carboxylic acid, tert-butyl ester (Intermediate 11; 0.54 g, 1.02 mmol), 2-bromo-1,1-diethoxyethane (0.31 mL) ethanol (20 mL) and 2N hydrochloric acid (3 drops) was heated under reflux for 5 hours. Additional aliquots of 2-bromo-1,1-diethoxyethane (0.31 mL) and 2N hydrochloric acid (6 drops) were added, and heating under reflux continued for a further 4 hours. The mixture was evaporated and the residue chromatographed on silica gel using firstly ethyl acetate-cyclohexane (1:2 v/v) as eluent, followed by further elution with ethyl acetate-methanol (1:1 v/v). The impure product was further purified by chromatography on an amine-capped solid phase extraction (SPE) cartridge, eluting firstly with dichloromethane to remove impurities and then with methanol-880 ammonia (1:1 v/v) as eluent to afford the title compound, a solid after crystallisation from acetonitrile. This was shown by nOe NMR spectroscopy to be inverted at the pyrrolidine C(4) centre relative to the starting material.

MS calcd for (C₂₆H₃₁N₃O₃S₂+H)⁺: 498. Found: (M+H)⁺=498.

¹H NMR (CD₃OD): δ 7.77 (1H, d), 7.45 (1H, d), 7.37 (1H, d), 7.23 (2H, d), 7.18 (2H, d), 7.11 (1H, d), 5.72 (1H, d), 4.30 (1H, m), 2.98 (1H, t), 2.69 (1H, dd), 2.57 (1H, br d), 2.06 (1H, dd), 1.99 (1H, m), 1.24 (9H, s), 1.86 (3H, d) and 1.02 (3H, d). Carboxylic acid proton exchanged with solvent. The corresponding rel-(2S,4S,5R)-diastereoisomer of the title compound was isolated from the acetonitrile crystallisation liquors and is described as Example 22 (below).

### Example 22

### rel-(2S,4S,5R)-1-(4-tert-butylbenzoyl)-2-isobutyl-4-(1,3-thiazol-2-yl)-5-(1,3-thiazol-2-yl)pyrrolidine-2-carboxylic acid

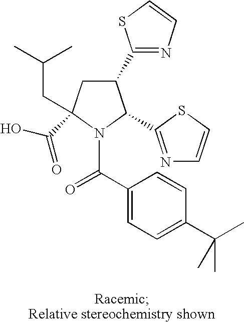

The crystallisation liquors from Example 21 (above) were purified by reverse phase HPLC on a C₁₈ column using a two solvent gradient elution with (A) water containing formic acid (0.1%) and (B) acetonitrile-water (95:5 v/v) containing formic acid (0.05%) as the eluents. Further purification by preparative TLC using ethyl acetate as eluent afforded the title compound, a solid.

MS calcd for (C₂₆H₃₁N₃O₃S₂+H)⁺: 498. Found: (M+H)⁺=498.

¹H NMR (CD₃OD): δ 7.69 (1H, d), 7.57 (1H, d), 7.36 (1H, d), 7.33 (1H, d), 7.28 (2H, d), 7.04 (2H, d), 5.98 (1H, d), 4.75 (1H, m), 3.05 (1H, t), 2.70 (1H, dd), 2.40-2.28 (2H, m), 2.12 (1H, m), 1.23 (9H, s), 1.14 (3H, d) and 1.18 (3H, d). Carboxylic acid proton exchanged with solvent.

### Example 23

### rel-(2S,4R,5R)-2-Isobutyl-1-(4-tert-butylbenzoyl)-4-(1,3,4-oxadiazol-2-yl)-5-(1,3-thiazol-2-yl)-pyrrolidine-2-carboxylic acid

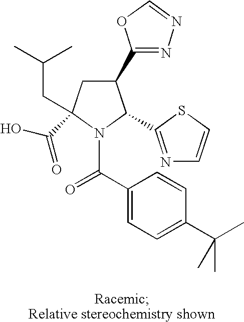

Stage A: A mixture of rel-(2S,4R,5R)-2-Isobutyl-1-(4-tert-butylbenzoyl)-4-hydrazinocarbonyl-5-(1,3-thiazol-2-yl)-pyrrolidine-2-carboxylic acid, tert-butyl ester and the corresponding rel-(2S,4S,5R)-diastereoisomer (Intermediate 14; 1.51g, 2.86 mmol) was dissolved in anhydrous triethyl orthoformate (100 mL) and heated at 125° C. for 30 hours, cooled and evaporated. The residue was chromatographed on silica gel using ethyl acetate-cyclohexane (1:3 v/v) as eluent.

Stage B: The early eluting crude rel-(2S,4R,5R)-diastereoisomer of the title compound tert-butyl ester (0.40 g, 0.74 mmol) was dissolved in trifluoroacetic acid (12 mL) and stirred at room temperature for 4.5 hours, then evaporated and re-evaporated twice from chloroform. The resulting material was crystallised from ethyl acetate-cyclohexane to afford the title compound, a solid.

MS calcd for (C₂₅H₃₀N₄O₄S+H)⁺: 483. Found: (M+H)⁺=483.

¹H NMR (CD₃OD): δ 8.89 (1H, s), 7.39 (1H, d), 7.25 (2H d), 7.23-7.17 (3H, m), 5.82 (1H, d), 4.24 (1H, m), 2.92 (1H, t), 2.76 (1H, dd), 2.53 (1H, br d), 2.06 (1H, dd), 1.94 (1H, m), 1.25 (9H, m), 1.17 (3H, d) and 1.00 (3H, d). Carboxylic acid proton exchanged with solvent.

The later eluting rel-(25,4S,5R)-diastereoisomer of the title compound tert-butyl ester from the aforementioned chromatography column (Stage A above) was similarly hydrolysed as described in Example 24 (below). Additional elution of the chromatography column (Stage A above) with ethyl acetate-cyclohexane (3:1 v/v) afforded an intermediate iminoether, described separately as Intermediate 15.

### Example 24

### rel-(2S,4S,5R)-2-Isobutyl-1-(4-tert-butylbenzoyl-4-(1,3,4-oxadiazol-2-yl)-5-(1,3-thiazol-2-yl)-pyrrolidine-2-carboxylic acid

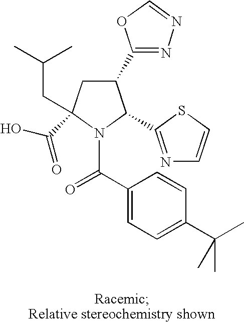

The later eluting crude title compound tert-butyl ester (0.30 g, 0.56 mmol) from the chromatography column in Example 23, Stage A was hydrolysed by treatment with trifluoroacetic acid in an analogous manner to that described in Example 23, Stage B to afford the title compound, a solid.

MS calcd for (C₂₅H₃₀N₄O₄S+H)⁺: 483. Found: (M+H)⁺=483.

¹H NMR (CD₃OD): δ 8.73 (1H, s), 7.63 (1H, d), 7.41 (1H, d), 7.30 (2H, d), 7.07 (2H, d), 6.12 (1H, d), 4.66 (1H, m), 3.11 (1H, t), 2.71 (1H, d), 2.35-2.27 (2H, m), 2.10 (1H, m), 1.24 (9H, s), 1.16 (3H, d) and 1.13 (3H, d). Carboxylic acid proton exchanged with solvent.

### Example 25

### rel-(2S,4R,5R)-2-Isobutyl-1-(4-tert-butylbenzoyl)-4-(1,3,4-thiadiazol-2-yl)-5-(1,3-thiazol-2-yl)-pyrrolidine-2-carboxylic acid

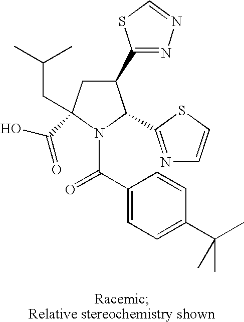

A solution of a mixture of rel-(2S,4R,5R)- and rel-(2S,4S,5R)-2-Isobutyl-1-(4-tert-butylbenzoyl)-4-(ethoxymethylene-hydrazinocarbonyl)-5-(1,3-thiazol-2-yl)-pyrrolidine-2-carboxylic acid, tert-butyl ester (Intermediate 15; 0.38 g, 0.65 mmol) and Lawesson's reagent (0.32 g, 0.78 mmol) in toluene (20 mL) was heated under reflux for 1.5 hours, cooled and evaporated. The residue was purified by reverse phase HPLC on a C₁₈ column using a two-solvent gradient elution with (A) water containing formic acid (0.1%) and (B) acetonitrile-water (95:5 v/v) containing formic acid (0.05%) as the eluents to afford the title compound, a solid, shown by nOe NMR studies to be the rel-(2S,4R,5R)-diastereoisomer.

MS calcd for (C₂₅H₃₀N₄O₃S₂+H)⁺: 499. Found (M+H)⁺=499.

¹H NMR (CD₃OD): δ 9.36 (1H, s), 7.40 (1H, d), 7.24 (2H, d), 7.20 (2H, d), 7.16 (1H, br d), 5.76 (1H, d), 4.49 (1H, m), 3.30 (1H, t), 2.82 (1H, dd), 2.56 (1H, br d), 2.08 (1H, dd), 1.98 (1H, m), 1.24 (9H, s), 1.19 (3H, d) and 1.02 (3H, d). Carboxylic acid proton exchanged with solvent.

### Example 26

### rel-(2R,4S,5R)-2-Benzyl-1-(4-tert-butylbenzoyl)-4-(1,2,4-oxadiazol-5-yl)-5-(1,3-thiazol-2-yl)-pyrrolidine-2-carboxylic acid

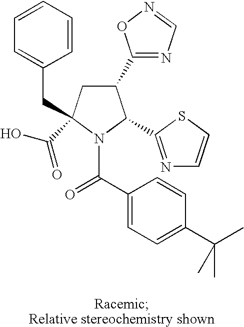

The title compound, a solid, was prepared from rel-(2R,4S,5R)-2-benzyl-4-(aminocarbonyl)-1-(4-tert-butylbenzoyl)-5-(1,3-thiazol-2-yl)-pyrrolidine-2-carboxylic acid, tert-butyl ester (Intermediate 18) in a manner analogous to that described for Example 6, and was shown by nOe NMR spectroscopy to be the desired rel-(2R,4S,5R)-diastereoisomer.

MS calcd for (C₂₈H₂₈N₄O₄S+H)⁺: 517. Found (M+H)⁺=517.

¹H NMR (CD₃OD): δ 8.20 (1H, s), 7.45-7.33 (5H, m), 7.24 (2H, d), 7.19 (2H, dd), 7.05 (2H, dd), 5.40 (1H, d), 3.94 (1H, d), 3.36 (1H, d), 3.06 (1H, dd), 2.93 (1H, m), 2.68 (1H, dd) and 1.13 (9H, s). Carboxylic acid proton exchanged with solvent.

### Example 27

### rel-(2S,4S,5R)-2-Isobutyl-1-(3-bromo-4-tert-butylbenzoyl)-4-(1-methyl-1H-tetrazol-5-yl)-5-(1,3-thiazol-2-yl)-pyrrolidine-2-carboxylic acid

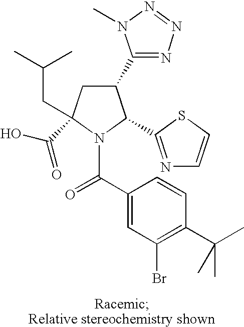

Reaction of rel-(2S,4S,5R)-2-isobutyl-4-(1-methyl-1H-tetrazol-5-yl)-5-(1,3-thiazol-2-yl)-pyrrol-idine-2-carboxylic acid, tert-butyl ester (Intermediate 19) with 3-bromo-4-tert-butylbenzoyl chloride in a manner analogous to that described for Intermediate 3 afforded the crude title compound tert-butyl ester. This was subsequently stirred with trifluoroacetic acid at room temperature overnight, evaporated and the residual material then purified by chromastography on silica gel using cyclohexane-ethyl acetate (gradient elution from 0:1 v/v to 1:10 v/v) as eluent to afford the title compound, a solid, confirmed by nOe NMR spectroscopy to be the rel-(2S,4S,5R)-diastereoisomer.

MS calcd for (C₂₅H₃₁BrN₆O₃S+H)⁺: 575/577. Found: (M+H)⁺=575/577

¹H NMR (CD₃OD): δ 7.60 (1H, d), 7.55 (2H, m), 7.30 (2H, m), 6.15 (1H, d), 4.70 (1H, m), 4.35 (3H, s), 3.45 (1H, m), 2.85 (1H, dd), 2.5 (1H, m), 2.40 (1H, m), 2.30 (1H, m), 1.60 (9H, s) and 1.30 (6H, m). Carboxylic acid proton exchanged with solvent.

### Example 28

### rel-(2S,4S,5R)-2-Isobutyl-1-(3-bromo-4-tert-butylbenzoyl)-4-(benzothiazol-1-2-yl)-5-(1,3-thiazol-2-yl)-pyrrolidine-2-carboxylic acid

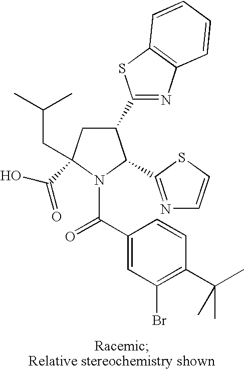

rel-(2S,4S,5R)-2-Isobutyl-4-(benzothiazol-2-yl)-5-(1,3-thiazol-2-yl)-pyrrolidine-2-carboxylic acid, tert-butyl ester (Intermediate 20) was acylated with 3-bromo-4-tert-butylbenzoyl chloride and subsequently subjected to ester hydrolysis with trifluoroacetic acid in a similar manner to that described for Example 27. The crude product was purified by reverse phase HPLC on a C₁₈ column using a two-solvent gradient elution with (A) water containing formic acid (0.1%) and (B) acetonitrile-water (95:5 v/v) containing formic acid (0.05%) as the eluents and the early fractions combined to afford the title compound, a solid, shown by nOe NMR studies to be the rel-(2S,4S,5R)-diastereoisomer.

MS calcd for (C₃₀H₃₂BrN₃O₃S₂+H)⁺: 626/628. Found: (M+H)⁺=626/628

¹H NMR (CD₃OD): δ 7.70 (2H, m), 7.60 (1H, d), 7.35 (2H, m), 7.25 (2H, m), 7.05 (2H, m), 5.95 (1H, d), 4.80 (1H, m), 3.10 (1H, t), 2.70 (1H, m), 2.25 (2H, m), 2.05 (1H, m), 1.35 (9H, s) and 1.05 (6H, m). Carboxylic acid proton exchanged with solvent.

Later eluting fractions from the preparative HPLC purification were combined to afford the corresponding rel-(2S,4R,5R)-diastereoisomer, described as Example 29 (below).

### Example 29

### rel-(2S,4R,5R)-2-Isobutyl-1-(3-bromo-4-tert-butylbenzoyl)-4-(benzothiazol-2-yl)-5-(1,3-thiazol-2-yl)-pyrrolidine-2-carboxylic acid

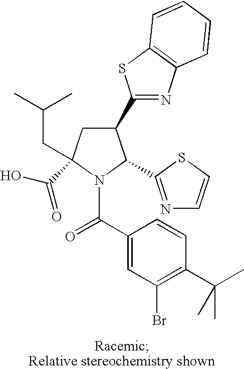

Later eluting fractions from the preparative HPLC purification of Example 28 were combined to afford the title compound, a solid, shown by nOe NMR studies to be the rel-(2S,4R,5R)-diastereoisomer, with configuration at the pyrrolidine C(4)-centre inverted relative to the starting material (Intermediate 20).

MS calcd for (C₃₀H₃₂BrN₃O₃S₂+H)⁺: 626/628. Found: (M+H)⁺=626/628

¹H NMR (CD₃OD): δ 8.00 (1H, d), 7.90 (1H, d), 7.50 (1H, t), 7.45 (1H, d), 7.40 (1H, t), 7.35 (1H, br), 7.30 (2H, m), 7.15 (1H, br), 5.85 (1H, d), 4.45 (1H, m), 3.10 (1H, t), 2.80 (1H, m), 2.60 (1H, m), 2.15 (1H, m), 2.00 (1H, m), 1.45 (9H, s), 1.25 (3H, d) and 1.05 (3H, d). Carboxylic acid proton exchanged with solvent.

### Example 30

### rel-(2S,4S,5R)-2-Isobutyl-1-(3-bromo-tert-butylbenzoyl)-4-(benzoxazol-2-yl)-5-(1,3-thiazol-2-yl)-pyrrolidine-2-carboxylic acid

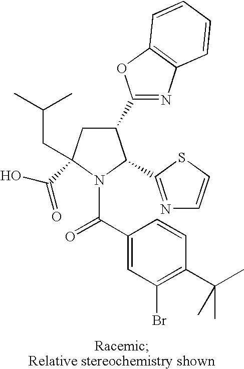

rel-(2S,4S,5R)-2-Isobutyl-4-(benzoxazol-2-yl)-5-(1,3-thiazol-2-yl)-pyrrolidine-2-carboxylic acid, tert-butyl ester (Intermediate 21) was acylated with 3-bromo-4-tert-butylbenzoyl chloride and subsequently subjected to ester hydrolysis with trifluoroacetic acid in a similar manner to that described for Example 27. The crude product was purified by reverse phase HPLC on a C₁₈ column using a two-solvent gradient elution with (A) water containing formic acid (0.1%) and (B) acetonitrile-water (95:5 v/v) containing formic acid (0.05%) as the eluents and the early fractions combined to afford the title compound, a solid, shown by nOe NMR studies to be the rel-(2S,4S,5R)-diastereoisomer.

MS calcd for (C₃₀H₃₂BrN₃O₄S+H)⁺: 610/612. Found: (M+H)⁺=610/612

H NMR (CD₃OD): δ 7.60 (1H, d), 7.50-7.45 (3H, m), 7.35-7.25 (3H, m), 7.20 (2H, m), 6.10 (1H, d), 4.70 (1H, m), 3.25 (1H, t), 2.75 (1H, m), 2.35 (2H, m), 2.15 (1H, m), 1.45 (9H, s) and 1.15 (6H, m). Carboxylic acid proton exhanged with solvent.

Later eluting fractions from the preparative HPLC purification were combined to afford the corresponding rel-(2S,4R,5R)-diastereoisomer, described as Example 29 (below).

### Example 31

### rel-(2S,4R,5R)-2-Isobutyl-1-(3-bromo-4-tert-butylbenzoyl-4-(benzoxazol-2-yl)-5-(1,3-thiazol-2-yl)-pyrrolidine-2-carboxylic acid

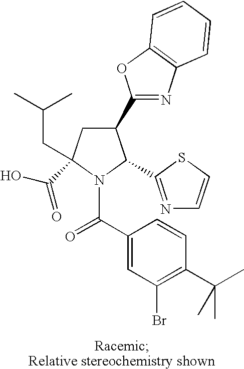

Later eluting fractions from the preparative HPLC purification of Example 30 were combined to afford the title compound, a solid, shown by nOe NMR studies to be the rel-(2S,4R,5R)-diastereoisomer, with configuration at the pyrrolidine C(4)-centre inverted relative to the starting material (Intermediate 21).

MS calcd for (C₃₀H₃₂BrN₃O₄S+H)⁺: 610/612. Found: (M+H)⁺=610/612

H NMR (CD₃OD): δ 7.65 (1H, m), 7.55 (1H, m), 7.50 (1H, d), 7.40-7.30 (5H, m), 7.20 (1H, br), 5.90 (1H, d), 4.30 (1H, m), 3.00 (1H, t), 2.80 (1H, m), 2.60 (1H, m), 2.10 (1H, m), 2.00 (1H, m), 1.45 (9H, s), 1.20 (3H, d) and 1.15 (3H, d). Carboxylic acid proton exchanged with solvent.

### Example 32

### rel-(2S,4S,5R)-1-(4-tert-butylbenzoyl)-2-isobutyl-4-(1,2,4-oxadiazol-5-yl)5-pyridin-2-yl-pyrrolidine-2-carboxylic acid

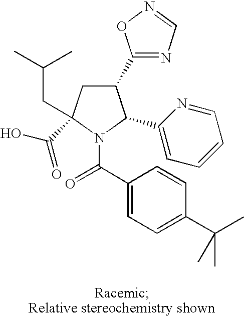

Stage A: A mixture of rel-(2S,4S,5R)-4-(aminocarbonyl)-2-isobutyl-5-pyridin-2-yl-pyrrolidine-2-carboxylic acid, tert-butyl ester (Intermediate 22; 0.443 g, 0.874 mmol) and (1,1-dimethoxymethyl)dimethylamine (20 mL) was heated at 120° C. for 5 hours and then concentrated. The residue was dissolved in a mixture of dioxan (5 mL) and acetic acid (5 mL) and to the resultant solution was added a solution of aqueous hydroxylamine (50% w/v, 75 uL). The mixture was heated at 90° C. for 2 hours and evaporated. The residue was dissolved in ethyl acetate and washed with water, dried (Na₂SO₄) and evaporated. The resulting foamy solid was purified firstly by chromatography on silica gel using cyclohexane-ethyl acetate (8:2 v/v) as eluent to remove the early eluting crude rel-(2S,4R,5R)-diastereoisomer of the title compound tert-butyl ester. The later fractions from the chromatography column were further purified by preparative TLC on silica gel using cyclohexane-ethyl acetate (4:6 v/v) as eluent to afford the crude title compound tert-butyl ester.

Stage B: The crude tert-butyl ester (100 mg) from the preparative TLC purification (above) was dissolved in trifluoroacetic acid (4 mL) and stirred at room temperature for 16 hours before being evaporated. The residue was triturated with diethyl ether to afford the title compound, a solid.

MS calcd for (C₂₇H₃₂N₄O₄+H)⁺: 477. Found: (M+H)⁺=477.

¹H NMR (CDCl₃): δ 8.53 (bd, 1H), 8.07 (s, 1H), 7.31 (dt, 1H), 7.21 (m, 1H) 7.13 (d, 2H), 6.91 (d, 2H), 6.30 (d, 1H), 5.59 (d, 1H), 4.51 (m, 1H), 3.20 (t, 1H), 2.63 (dd, 1H), 2.55 (dd, 1H), 2.39 (dd, 1H), 2.00 (m, 1H), 1.22 (d, 3H), 1.20 (s, 9H), 1.14 (d, 3H). Carboxylic acid proton not seen.

The early eluting crude rel-(2S,4R,5R)-diastereoisomer of the title compound tert-butyl ester from the chromatograph column described in Stage A (above) was similarly hydrolysed as described in Example 33 (below).

### Example 33

### rel-(2S,4R,5R)-1-(4-tert-butylbenzoyl-2-isobutyl-4-(1,2,4-oxadiazol-5-yl)-5-pyridin-2-yl-pyrrolidine-2-carboxylic acid

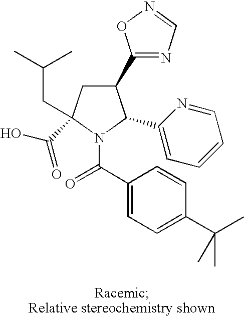

The earlier eluting crude title compound tert-butyl ester from the chromatography column in Example 32, Stage A was hydrolysed by treatment with trifluoroacetic acid in an analogous manner to that described in Example 32, Stage B to afford the title compound, a solid, which was confirmed by nOe NMR spectroscopy to be the rel-(2S,4R,5R)-diastereoisomer.

MS calcd for (C₂₇H₃₂N₄O₄+H)⁺: 477. Found: (M+H)⁺=477.

¹H NMR(CDCl₃): δ 8.46 (bd, 1H), 8.38 (s,1H),7.29 (dt, 1H),7.16 (d,2H), 7.11 (m, 1H), 7.06 (d, 2H), 6.55 (d, 1H), 5.56 (d, 1H), 3.99 (m, 1H), 3.11 (dd, 1H), 2.73 (t, 1H), 2.42 (d, 2H), 1.88 (m, 1H), 1.23 (s, 9H), 1.12 (d, 3H), 1.03 (d, 3H). Carboxylic acid proton not seen.

### Example 34

### Enantiomer A of rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid

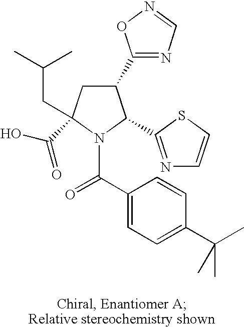

The title compound was prepared by sequentially following procedures analogous to those described for Intermediate 3 and Example 6, but utilising Enantiomer A of rel-(2S,4S,5R)-4-(aminocarbonyl)-2-isobutyl-5-(1,3-thiazol-2-yl)pyrrolidine-2-carboxylic acid, tert-butyl ester (Intermediate 23) in place of the corresponding racemate. This compound was identical by 1H NMR and MS to the racemic title compound described in Example 6.

### Example 35

### Enantiomer A of rel-(2S,4S,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid

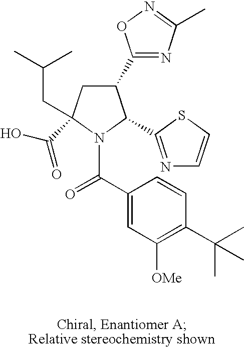

Enantiomer A of the title compound was prepared from Enantiomer A of rel-(2S,4S,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 24) in a similar manner to that described for Example 27, but substituting 3-methoxy-4-tert-butylbenzoyl chloride in place of 3-bromo-4-tert-butylbenzoyl chloride. The title compound was confirmed by nOe NMR spectroscopy to be the rel-(2S,4S,5R)-diastereoisomer.

MS calcd for (C₂₇H₃₄N₄O₅S+H)⁺: 527. Found: (M+H)⁺=527.

¹H NMR (CD₃OD): δ 7.70 (1H, d), 7.40 (1H, d), 7.20 (1H, d), 6.80 (1H, dd), 6.40 (1H, br), 6.10 (1H, d), 4.60 (1H, m), 3.60 (3H, s), 3.10 (1H, t), 2.70 (1H, m), 2.30 (2H, m), 2.10 (3H, s), 2.05 (1H, m), 1.30 (9H, s) and 1.20-1.10 (6H, m). Carboxylic acid proton exchanges with solvent.

### Example 36

### Enantiomer A of rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid

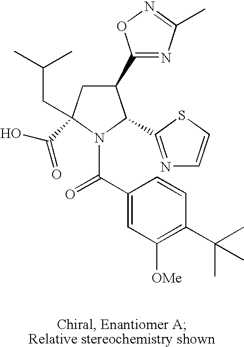

Enantiomer A of the title compound was prepared from Enantiomer A of rel-(2S,4R,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 25) in a similar manner to that described for Example 27, but substituting 3-methoxy-4-tert-butylbenzoyl chloride in place of 3-bromo-4-tert-butylbenzoyl chloride.

MS calcd for (C₂₇H₃₄N₄O₅S+H)⁺: 527. Found: (M+H)⁺=527.

¹H NMR (CD₃OD): δ 7.45 (1H, d), 7.30 (1H, d), 7.20 (1H, d), 6.90 (1H, d), 6.65 (1H, br), 5.85 (1H, d), 4.25 (1H, m), 3.75 (3H, s), 2.90 (1H, t), 2.80 (1H, m), 2.50 (1H, m), 2.35 (3H, s), 2.10 (1H, m), 1.95 (1H, m), 1.30 (9H, s), 1.20 (3H, d) and 1.00 (3H, d). Carboxylic acid proton exchanges with solvent.

### Example 37

### Enantiomer A of rel-(2S,4S,5R)-1-(3-chloro-4-tert-butylbenzoyl)-2-isobutyl-4-(3-methyl-1,2,4-oxadiazole-5-yl)-5-(1,3-thiazol-2-yl)pyrrolidine-2-carboxylic acid

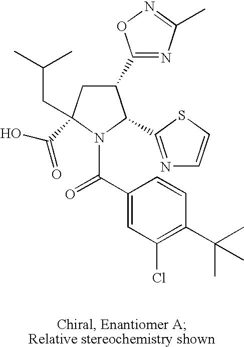

Enantiomer A of the title compound was prepared from Enantiomer A of rel-(2S,4S,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 24) in a similar manner to that described for Example 27, but substituting 3-chloro-4-tert-butylbenzoyl chloride in place of 3-bromo-4-tert-butylbenzoyl chloride. The title compound was confirmed by nOe NMR spectroscopy to be the rel-2S,4S,5R)-diastereoisomer.

MS calcd for (C₂₆H₃₁ClN₄O₄S+H)⁺: 531/533. Found: (M+H)⁺=531/533.

¹H NMR (CD₃OD): δ 7.70 (1H, d), 7.50 (1H, d), 7.40 (1H, d), 7.10 (1H, dd), 7.00 (1H, br), 6.10 (1H, d), 4.60 (1H, m), 3.05 (1H, t), 2.70 (1H, m), 2.30 (2H, m), 2.10 (3H, s), 2.05 (1H, m), 1.40 (9H, s) and 1.10 (6H, m). Carboxylic acid proton exchanged with solvent.

### Example 38

### Enantiomer A of rel-(2S,4R,5R)-1-(3-chloro-4-tert-butylbenzoyl)-2-isobutyl-4-(3-methyl-1,2,4-oxadiazol-5-yl)-5-(1,3-thiazol-2-yl)pyrrolidine-2-carboxylic acid

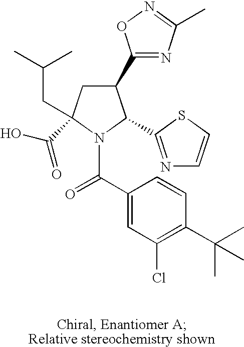

Enantiomer A of the title compound was prepared from Enantiomer A of rel-(2S,4R,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 25) in a similar manner to that described for Example 27, but substituting 3-chloro-4-tert-butylbenzoyl chloride in place of 3-bromo-4-tert-butylbenzoyl chloride.

MS calcd for (C₂₆H₃₁ClN₄O₄S+H)⁺: 531/533. Found: (M+H)⁺=531/533.

¹H NMR (CD₃OD): δ 7.50 (1H, d), 7.40 (1H, d), 7.20 (2H, m), 7.10 (1H, br), 5.80 (1H, d), 4.20 (1H, m), 2.90 (1H, t), 2.80 (1H, m), 2.50-2.40 (1H, br), 2.30 (3H, s), 2.10 (1H, m), 1.90 (1H, m), 1.40 (9H, s), 1.15 (3H, d) and 1.01 (3H, d). Carboxylic acid proton exchanges with solvent.

### Example 39

### Enantiomer A of rel-(2S,4R,5R)-2-isobutyl-1-(4-tert-butylbenzoyl)-4-(3-methyl-1,2,4-oxadiazol-5-yl)-5-(1,3-thiazol-2-yl)pyrrolidine-2-carboxylic acid

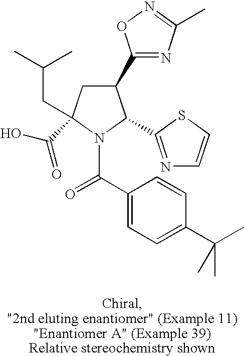

Enantiomer A of the title compound was prepared from Enantiomer A of rel-(2S,4R,5R)-2-isobutyl-4-(3-methyl-1,2,4-oxadiazol-5-yl)-5-(1,3-thiazol-2-yl)pyrrolidine-2-carboxylic acid, tert-butyl ester (Intermediate 25) in a similar manner to that described for Example 27, but substituting 4-tert-butylbenzoyl chloride in place of 3-bromo-4-tert-butylbenzoyl chloride. This material was spectroscopically identical to the corresponding racemate, described as Example 4 and also corresponds to the 2^{nd} eluting enantiomer described in Example 11.

### Example 40

### Enantiomer A of rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxamide

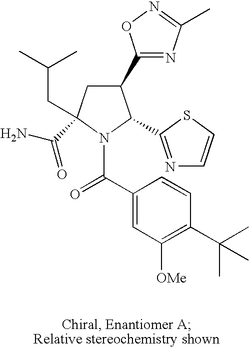

Enantiomer A of the title compound, a solid, was prepared from Enantiomer A of rel-(2S,4R,5R)-2-isobutyl-1-(3-methoxy-4-tert-butylbenzoyl)-4-3-methyl-1,2,4-oxadiazol-5-yl)-5-(1,3-thiazol-2yl)pyrrolidine-2-carboxylic acid (Example 36) in a manner analogous to that described in Example 15.

MS calcd for (C₂₇H₃₅N₅O₄S+H)⁺: 526. Found: (M+H)⁺=526.

¹H NMR (CD₃OD): δ 7.65 (1H, d), 7.35 (1H, d), 7.20 (1H, d), 6.95 (1H, d), 6.45 (1H, br), 5.90 (1H, d), 4.05 (1H, m), 3.70 (3H, s), 2.90 (1H, m), 2.80 (1H, m), 2.50 (1H, m), 2.35 (3H, s), 2.30 (1H, m), 1.95 (1H, m), 1.30 (9H, s), 1.20 (3H, d) and 1.00 (3H, d). Amide protons exchange with solvent.

### Example 41

### rel-(2S,4R,5R)-2-Isobutyl-1-(3-methoxy-4-tert-butylbenzoyl)-4-(5-methyl-1,2,4oxadiazol-3-yl)-5-(1,3-thiazol-2-yl)pyrrolidine-2-carboxylic acid

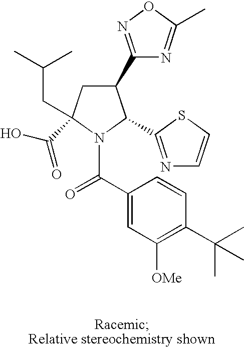

rel-(2S,4R,5R)-2-Isobutyl-4-(5-methyl-1,2,4-oxadiazol-3-yl)-5-(1,3-thiazol-2-yl)pyrrolidine-2-carboxylic acid, tert-butyl ester (Intermediate 28) was acylated with 3-methoxy-4-tert-butylbenzoyl chloride and subsequently subjected to ester hydrolysis with trifluoroacetic acid in a similar manner to that described for Example 27. The resulting oil was tritutated with diethyl ether to afford the title compound, a solid.

MS calcd for (C₂₇H₃₄N₄O₅S+H)⁺: 527. Found: (M+H)+=527.

¹H NMR (CD₃OD): δ 7.40 (1H, d), 7.20-7.10 (2H, m), 6.90 (1H, d), 6.60 (1H, br), 5.75 (1H, d), 4.05 (1H, m), 3.75 (3H, s), 3.50 (1H, dd), 2.90 (1H, t), 2.65 (1H, dd), 2.60 (3H, s), 2.10 (1H, m), 2.00 (1H, m), 1.30 (9H, s), 1.20 (3H, d) and 1.05 (3H, d). Carboxylic acid proton exchanges with solvent.

### Example 42

### rel-(2S,4S,5R)-2-Isobutyl-1-(3-methoxy-4-tert-butylbenzoyl)-4-(5-methyl-1,2,4oxadiazol-3-yl)-5-(1,3-thiazol-2-yl)pyrrolidine-2-carboxylic acid

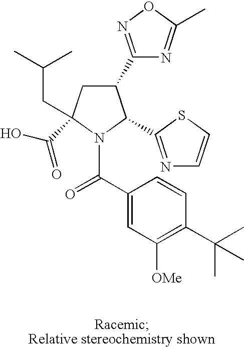

rel-(2S,4S,5R)-2-Isobutyl-4-(5-methyl-1,2,4-oxadiazol-3-yl)-5-(1,3-thiazol-2-yl)pyrrolidine-2-carboxylic acid, tert-butyl ester (Intermediate 29) was acylated with 3-methoxy-4-tert-butylbenzoyl chloride and subsequently subjected to ester hydrolysis with trifluoroacetic acid in a similar manner to that described for Example 27. The resulting oil was tritutated with diethyl ether to afford the title compound, a solid.

MS calcd for (C₂₇H₃₄N₄O₅S+H)⁺: 527. Found: (M+H)⁺=527.

¹H NMR (CD₃OD): δ 7.75 (1H, d), 7.45 (1H, d), 7.25 (1H, d), 6.80 (1H, d), 6.45 (1H, s), 6.00 (1H, m), d), 4.45 (1H, m), 3.65 (3H, s), 3.00 (1H, t), 2.60 (1H, dd), 2.40 (3H, s), 2.35 (2H, m), 1.30 (9H, s), 1.20 (3H, d) and 1.15 (3H, d). Carboxylic acid proton exchanges with sovent.

### Example 43

### Enantiomer A of rel-(2S,4S,5R)-5-(benzothiazol-2-yl)-2-isobutyl-1-(4-tert-butylbenzoyl)-4-(1,2,4-oxadiazol-5-yl)-pyrrolidine2-carboxylic acid

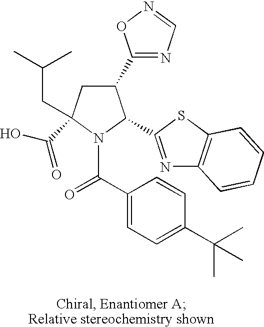

The Enantiomer A of the title compound, a solid, was prepared by analogy with the method described in Example 6, and using Enantiomer A of rel-(2S,4S,5R)-4-(aminocarbonyl)-2-isobutyl-1-(4-tert-butylbenzoyl)-5-(benzothiazol-2-yl)pyrrolidine-2-carboxylic acid, tert-butyl ester (Intermediate 32) a starting material.

MS calcd for (C₂₉H₃₂N₄O₄S+H)⁺: 533. Found: (M+H)⁺=533.

¹H NMR (CD₃OD): δ 8.25 (1H, s), 7.75 (2H, m), 7.45 (1H, m), 7.35 (1H, m), 7.15 (2H, d), 7.05 (2H, d), 6.20 (1H, d), 4.75 (1H, m), 3.15 (1H, t), 2.70 (1H, dd), 2.30 (2H, dd), 2.10 (1H, m) and 1.10-1.00 (15H, m). Carboxylic acid proton exchanges with solvent.

### Example 44

### Enantiomer A of rel-(2S,4S,5R)-5-(benzothiazol-2-yl)-2-isobutyl-1-(3-bromo-4-tert-butyl-benzoyl)-4-(1,2,4-oxadiazol-5-yl)-pyrrolidine-2-carboxylic acid

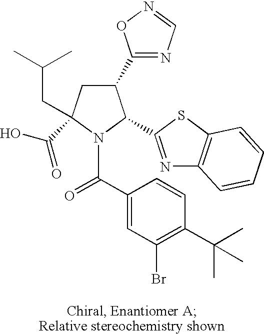

The Enantiomer A of the title compound, a solid, was prepared by acylation of Enantiomer A of rel-(2S,4S,5R)-4-(aminocarbonyl)-2-isobutyl-5-(benzothiazol-2-yl)pyrrolidine-2-carboxylic acid, tert-butyl ester (Intermediate 31) with 3-bromo-4-tert-butylbenzoyl chloride in a similar manner to that described in Intermediate 3 and subsequently following a procedure analogous to that outlined in Example 6 for conversion of the amide group into the corresponding 1,2,4-oxadiazole.

MS calcd for (C₂₉H₃₁BrN₄O₄S+H)⁺: 611/613. Found: (M+H)⁺=611/613.

¹H NMR (CD₃OD): δ 8.30 (1H, s), 7.90 (1H, d), 7.80 (1H, d), 7.50 (1H, m), 7.45 (1H, m), 7.35 (1H, d), 7.20 (1H, d), 7.10 (1H, s), 6.10 (1H, d), 4.80 (1H, m), 3.25 (1H, t), 2.80 (1H, dd), 2.35 (2H, m), 2.10 (1H, m), 1.40 (9H, s) and 1.20 (6H, m). Carboxylic acid proton exchanges with solvent.

### Example 45

### rel-(2S,4S,5R)-1-(3-Bromo-4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid

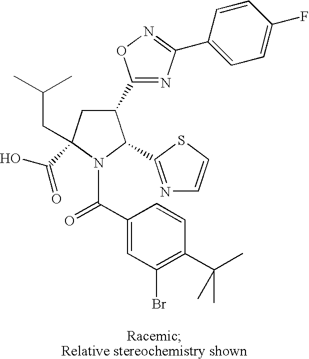

Stage A: rel-(2S,4S,5R)-4-[3-(4-fluorophenyl)-1,2,4-oxadiazol-5-yl]-2-isobutyl-5-(1,3-thiazol-2-yl)pyrrolidine-2-carboxylic acid, tert-butyl ester (Intermediate 35) was acylated with 3-bromo-4-tert-butylbenzoyl chloride in a manner similar to that described in Intermediate 3. The crude reaction mixture was purified by chromatography on silica gel using cyclohexane-ethyl acetate (7:1 v/v) to afford the title compound, tert-butyl ester, an oil.

Stage B: The tert-butyl ester was dissolved in trifluoroacetic acid and stirred at room temperature for 3.5 hours, evaporated, repeatedly re-evaporated from dichloromethane and then triturated with diethyl ether to afford the title compound, a solid.

MS calcd for (C₃₁H₃₂BrFN₄O₄S+H)⁺: 655/657. Found: (M+H)⁺=655/657.

¹H NMR (DMSO-d₆): δ 7.83 (2H, m), 7.54 (1H, d), 7.48 (1H, d), 7.33 (2H, t), 7.34 (1H, br), 7.27 (1H, br), 7.14 (1H, br), 6.14 (1H, d), 4.68 (1H, m), 2.98 (1H, t), 2.74 (1H, dd), 2.27-2.07 (2H, m), 2.20 (1H, m), 1.39 (9H, s) and 1.05 (6H, d). Carboxylic acid proton exchanges with solvent.

### Example 46

### rel-(2S,4S,5R)-1-(4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid

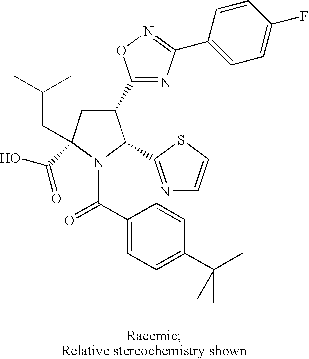

The title compound, a solid, was prepared from rel-(2S,4S,5R)-4-[3-(4-fluorophenyl)-1,2,4-oxadiazol-5-yl]-2-isobutyl-5-(1,3-thiazol-2-yl)pyrrolidine-2-carboxylic acid, tert-butyl ester (Intermediate 35) and 4-tert-butylbenzoyl chloride in a similar manner to that described in Example 27.

MS calcd for (C₃₁H₃₃FN₄O₄S+H)⁺: 577. Found: (M+H)⁺=577.

¹H NMR (DMSO-d₆): δ 7.83 (2H, m), 7.49 (1H, d), 7.46 (1H, d), 7.32 (2H, t), 7.25 (2H, d) 7.11 (2H, d), 6.19 (1H, d), 4.68 (1H, m), 2.96 (1H, t), 2.70 (1H, dd), 2.22-2.09 (2H, m) 2.04 (1H, m), 1.19 (9H, s) and 1.05 (6H, t). Carboxylic acid proton exchanges with solvent.

### Example 47

### Enantiomer A of rel-(2S,4S, 5R)-1-(3-bromo-4-tert-butylbenzoyl)-4-[3-(4fluorophenyl)-1,2,4-oxadiazol-5-yl]-2-isobutyl-5-(1,3-thiazol-2-yl)pyrrolidine-2-carboxylic acid

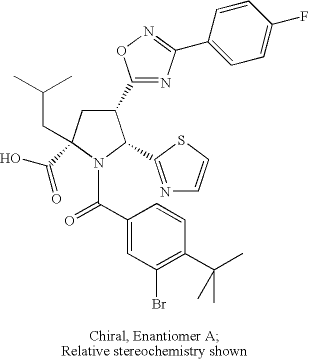

Stage A: rel-(2S,4S,5R)-1-(3-bromo-4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid, tert-butyl ester (from Example 45, Stage A) was resolved by preparative HPLC on a Chiralpak AD chromatography column using heptane-isopropanol (85:15 v/v) as eluent to afford Enantiomer A (>95% ee) and Enantiomer B (>95% ee) of the tert-butyl ester with retention times of 5.81 and 8.99 minutes respectively.

Stage B: Enantiomer A of the tert-butyl ester (above) was treated with trifluoroacetic acid in an analogous manner to that described in Example 45, Stage B to afford Enantiomer A of the title compound, a solid.

MS calcd for (C₃₁H₃₂BrFN₄O₄S+H)⁺: 655/657. Found: (M+H)⁺=655/657.

¹H NMR (CDCl₃): δ 7.88 (2H, m), 7.83 (1H, d), 7.32 (1H, d), 7.24 (1H, d), 7.12 (2H, t), 7.07 (1H, d), 6.96 (1H, dd), 5.74 (1H, dd), 4.46 (1H, m), 3.25 (1H, t), 2.72 (1H, dd), 2.47 (1H, dd), 2.41 (1H, dd), 1.98 (1H, m), 1.43 (9H, s), 1.19 (3H, d) and 1.15 (3H, d). Carboxylic acid proton not seen.

### Example 48

### rel-(2S,4R,5R)-1-(4-tert-Butylbenzoyl)-4-[3-bromo-1,2,4-thiadiazol-5-yl]-2-isobutyl-5-(1,3-thiazol-2-yl)pyrrolidine-2-carboxylic acid

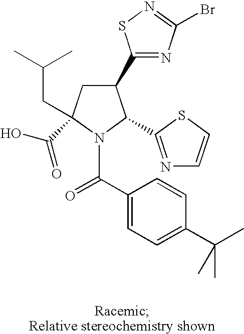

The title compound, a solid, was prepared from rel-(2S,4R,5R)-4-[3-bromo-1,2,4thiadiazol-5-yl]-2-isobutyl-5-(1,3-thiazol-2-yl)pyrrolidine-2-carboxylic acid, tert butyl ester (Intermediate 37) and 4-tert-butylbenzoyl chloride in a similar manner to that described in Example 45.

MS calcd for (C₂₅H₂₉BrN₄O₃S₂+H)⁺: 577/579. Found: (M+H)⁺=577/579.

¹H NMR (CDCl₃): δ 7.49 (1H, d), 7.27 (2H, d), 7.19 (2H, d), 7.10 (1H, d), 5.72 (1H, d), 4.16 (1H, m), 3.41 (1H, d), 2.71 (1H, dd), 2.62 (1H, t), 1.96 (1H, dd), 1.89 (1H, m), 1.26 (9H, s), 1.26 (9H, s), 1.07 (3H, d) and 1.04 (3H, d). Carboxylic acid proton not seen.

### Example 49

### rel-(2S,4S,5R)-1-(4-tert-butylbenzoyl)-4-(3-bromo-1,2,4-thiadiazol-5-yl)-2-isobutyl-5-(1,3-thiazol-2-yl)pyrrolidine-2-carboxylic acid

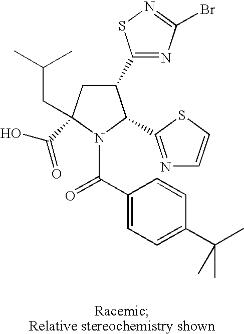

The title compound, a solid, was prepared from rel-(2S,4S,5R)-4-[3-bromo-1,2,4-thiadiazol-5-yl]-2-isobutyl-5-(1,3-thiazol-2-yl)pyrrolidine-2-carboxylic acid, tert butyl ester (Intermediate 38) and 4-tert-butylbenzoyl chloride in a similar manner to that described in Example 45.

MS calcd for (C₂₅H₂₉BrN₄O₃S₂+H)⁺: 577/579. Found: (M+H)⁺=577/579.

¹H NMR (DMSO-d₆): δ 13.87 (1H, br), 7.64 (1H, d), 7.54 (1H, d), 7.26 (2H, d), 6.99 (2H, d), 6.10 (1H, d), 4.94 (1H, m), 2.90 (1H, t), 2.63 (1H, dd), 2.10-2.24 (2H, m), 2.04 (1H, m), 1.20 (9H, s) 1.08 (3H, d) and 1.04 (3H, d).

### Example 50

### rel-(2S,4S,5R)-2-Isobutyl-1-(3-methoxy-4-tert-butylbenzoyl)-4-(3-methyl-1,2,4-oxadiazol-5-yl)-5-thien-2-ylpyrrolidine-2-carboxylic acid

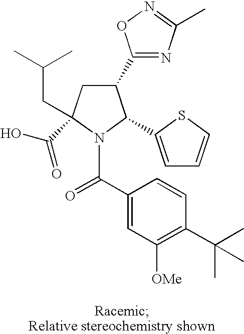

rel-(2S,4S,5R)-2-Isobutyl-4-(3-methyl-1,2,4-oxadiazol-5-yl)-5-thien-2-ylpyrrolidine-2-carboxylic acid tert-butyl ester (Intermediate 39) was acylated with 3-methoxy-4-tert-butylbenzoyl chloride and subsequently subjected to ester hydrolysis with trifluoroacetic acid in a similar manner to that described for Example 27. The resulting oil was triturated with diethyl ether to afford the title compound, a solid.

MS calcd for (C₂₈H₃₅N₃O₅S+H)⁺: 526. Found: (M+H)⁺=526.

¹H NMR (CD₃OD): δ 7.20 (1H, d), 7.10 (1H, d), 7.00 (1H, d), 6.80 (1H, d), 6.70 (1H, dd), 6.40 (1H, s), 5.80 (1H, d), 4.45-4.50 (1H, m), 3.60 (3H, s), 3.20 (1H, t), 2.60-2.70 (1H, dd) 2.35-2.40 (1H, dd), 2.20 (1H, m), 2.15 (3H, s), 2.10 (1H, m), 1.30 (9H, s) and 1.15 (6H, dd). Carboxylic acid proton exchanges with solvent.

### Example 51

### rel-(2S,4R,5R)-2-Isobutyl-1-(3-methoxy-4-tert-butylbenzoyl)-4-(3-methyl-1,2,4-oxadiazol-5-yl)-5-thien-2-ylpyrrolidine-2-carboxylic acid

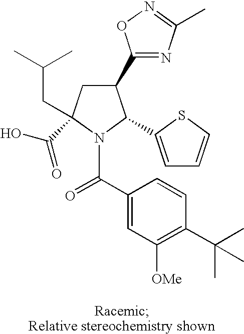

rel-(2S,4R,5R)-2-Isobutyl-4-(3-methyl-1,2,4-oxadiazol-5-yl)-5-thien-2-ylpyrrolidine-2-carboxylic tert-butyl ester (Intermediate 40) was acylated with 3-methoxy-4-tert-butylbenzoyl chloride and subsequently subjected to ester hydrolysis with trifluoroacetic acid in a similar manner to that described for Example 27 to afford the title compound, a foam.

MS calcd for (C₂₈H₃₅N₃O₅S+H)⁺: 526. Found: (M+H)⁺=526.

¹H NMR (CD₃OD): δ 7.15 (2H, m), 6.90 (1H, d), 6.50-6.55 (2H, m), 6.45 (1H, s), 5.65 (1H, d), 4.15-4.20 (1H, m), 3.70 (3H, s), 2.65-2.80 (2H, m), 2.55-2.60 (1H, dd), 2.35 (3H, s), 2.00-2.05 (1H, m), 1.90-2.00 (1H, m), 1.30 (9H, s), 1.20 (3H, d) and 1.05 (3H, d). Carboxylic acid proton exchange with solvent.

### Example 52

### rel-(2S,4R,5R)-2-Isobutyl-1-(3-methoxy-4-tert-butylbenzoyl)-4-methyl-4-(5-methyl-1,2,4-oxadiazol-3-yl)-5-(1,3-thiazol-2-yl)pyrrolidine-2-carboxylic acid

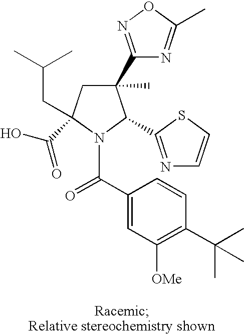

rel-(2S,4R,5R)-2-Isobutyl-4-methyl-4-(5-methyl-1,2,4-oxadiazol-3-yl)-5-(1,3-thiazol-2-yl)pyrrolidine-2-carboxylic acid tert-butyl ester (Intermediate 42) was acylated with 3-methoxy-4-tert-butylbenzoyl chloride and subsequently subjected to ester hydrolysis with trifluoroacetic acid in a similar manner to that described for Example 27 to afford the title compound, a solid.

MS calcd for (C₂₈H₃₀N₄O₅S+H)⁺: 541. Found: (M+H)⁺=541.

¹H NMR (CD₃OD): δ 8.00 (1H, d), 7.80 (1H, d), 7.40 (1H, d), 7.00 (1H, d), 6.95 (1H, s), 6.65 (1H, s), 3.95 (3H, s), 3.10 (1H, d), 2.85-2.90 (4H, m), 2.20 (1H, m), 2.00 (1H, m) 1.70 (1H, dd), 1.50 (9H, s), 1.25 (3H, s), 1.10 (3H, d) and 1.00 (3H, d). Carboxylic acid proton exchanges with solvent.

### Example 53

### rel-(2S,4S,5R)-2-Isobutyl-1-(3-methoxy-4-tert-butylbenzoyl)-4-(5methyl-1,2,4-oxadiazol-3-yl)-5-thien-2-ylpyrrolidine-2-carboxylic acid

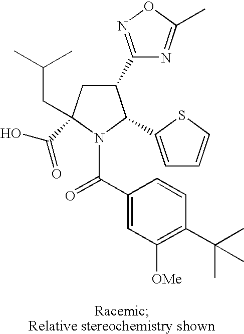

rel-(2S,4S,5R)-2-Isobutyl-4-(5-methyl-1,2,4-oxadiazol-3-yl)-5-thien-2-ylpyrrolidine-2-carboxylic acid, tert-butyl ester (Intermediate 46) was acylated with 3-methoxy-4-tert-butylbenzoyl chloride and subsequently subjected to ester hydrolysis with trifluoroacetic acid in a similar manner to that described for Example 27 to afford the title compound, a solid.

MS calcd for (C₂₈H₃₅N₃O₅S+H)⁺: 526. Found: (M+H)⁺=526.

¹H NMR (CD₃OD): δ 7.20 (1H, d), 7.05 (1H, d), 6.95 (1H, d), 6.80 (1H, d), 6.70 (1H, dd), 6.40 (1H, s), 5.65 (1H, d), 4.20-4.30 (1H, m), 3.60 (3H, s), 3.10-3.20 (1H, t), 2.55-2.60 (1H, dd), 2.40 (4H, m), 2.22-2.25 (1H, dd), 2.10 (1H, m), 1.30 (9H, s) and 1.15 (6H, dd). Carboxylic acid proton exchanges with solvent.

### Example 54

### rel-(2S,4R,5R)-2-Isobutyl-1-(3-methoxy-4-tert-butylbenzoyl)-4-(5-methyl-1,2,4-oxadiazol-3-yl)-5-thien-2-ylpyrrolidine-2-carboxylic acid

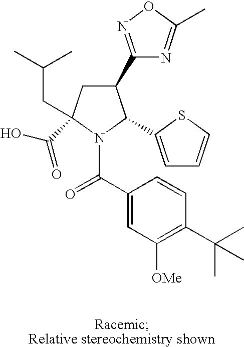

rel-(2S,4R,5R)-2-Isobutyl-4-(5-methyl-1,2,4-oxadiazol-3-yl)-5-thien-2-ylpyrrolidine-2-carboxylic acid, tert-butyl ester (Intermediate 45) was acylated with 3-methoxy-4-tert-butylbenzoyl chloride and subsequently subjected to ester hydrolysis with trifluoroacetic acid in a similar manner to that described for Example 27 to afford the title compound, a solid.

MS calcd for (C₂₈H₃₅N₃O₅S+H)⁺: 526. Found: (M+H)⁺=526.

¹H NMR (CD₃OD): δ 7.15 (1H, d), 7.10 (1H, d), 6.90 (1H, d), 6.55 (1H, s), 6.50 (1H, dd), 6.40 (1H, d), 5.60 (1H, d), 4.00 (1H, m), 3.70 (3H, s), 2.72 (1H, t), 2.50-2.60 (5H, m), 1.95-2.05 (2H, m), 1.30 (9H, s), 1.20 (3H, d)and 1.05 (3H, d). Carboxylic acid proton exchanges with solvent.

### Example 55

### rel-(2S,4S,5R)-2-Isobutyl-1-(4-tert-butylbenzoyl)-4-(3-methyl-1,2,4-oxadiazol-5-yl)-5-pyridin-3-ylpyrrolidine-2-carboxylic acid, trifluoroacetate salt

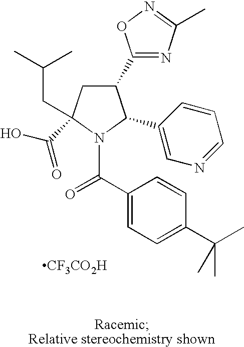

Stage A: 4-tert-Butylbenzoyl chloride (0.089 g, 0.451 mmol) was added to a solution of rel-(2S,4S,5R)-2-isobutyl-4-(3-methyl-1,2,4-oxadiazol-5-yl)-5-pyridin-3-ylpyrrolidine-2-carboxylic acid, tert-butyl ester (Intermediate 47; 0.145 g, 0.376 mmol) in dry dichloromethane (5 mL). Triethylamine (70 mL, 0.47 mmol) was added and the mixture heated at reflux for 18 hours, cooled and then partitioned between dichloromethane and saturated aqueous sodium bicarbonate solution. The dichloromethane solutions were combined and washed with brine, dried (Na₂SO₄) and evaporated to an oil. This was chromatographed on silica gel using cyclohexane-ethyl acetate (3:2 v/v) as eluent to afford the title compound tert-butyl ester as a foam.

Stage B: The tert-butyl ester (0.070 g, 0.128 mmol) was dissolved in trifluoroacetic acid (2 mL), stirred at room temperature for 4 hours, then evaporated. The residue was suspended in aqueous methanol, charged onto a reverse phase solid phase extraction cartridge and the cartridge was then eluted firstly with water to remove impurities and then with methanol-acetonitrile (1:1 v/v). The product containing fractions were evaporated to afford the title compound trifluoroacetate salt as a solid.

MS calcd for parent free base (C₂₈H₃₄N₄O₄+H)⁺: 491. Found: (M+H)⁺=491.

¹H NMR (CD₃OD): δ 8.20 (1H, s), 8.15 (1H, d), 7.80 (1H d), 7.20 (2H, d), 7.00-7.05 (3H, m), 5.75 (1H, d), 4.60-4.70 (1H, m), 3.10-3.20 (1H, t), 2.70 (1H, dd), 2.37 (1H, m), 2.28 (1H, m), 2.12 (1H, m), 2.05 (3H, s), 1.25 (9H, s) and 1.15 (6H, d). Carboxylic acid protons exchange with solvent.

¹⁹F NMR (CD₃OD): δ −77.4 (s).

### Example 56

### Enantiomer A of rel-(2S,4S,5R)-2-Isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid

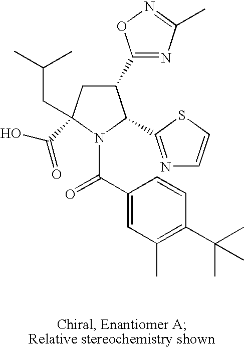

Enantiomer A of the title compound was prepared from Enantiomer A of rel-(2S,4S,5R)-2-isobutyl-4-3-methyl-1,2,4-oxadiazol-5-yl)-5-(1,3-thiazol-2-yl)pyrrolidine-2-carboxylic acid, tert-butyl ester (Intermediate 24) in a similar manner to that described for Example 27, but substituting 3-methyl-4-tert-butylbenzoyl chloride in place of 3-bromo-4-tert-butylbenzoyl chloride. The title compound was confirmed by nOe NMR spectroscopy to be the rel-(2S,4S,5R)-diastereoisomer.

MS calcd for (C₂₇H₂₉N₄O₄S+H)⁺: 511. Found: (M+H)⁺=511.

¹H NMR (CD₃OD): δ 7.70 (1H, d), 7.45 (1H, d), 7.30 (1H, d), 6.95 (1H, d), 6.75 (1H, s), 6.10 (1H, d), 4.65 (1H, m), 3.10 (1H, t), 2.70 (1H, dd), 2.40 (3H, s), 2.30-2.35 (2H, m), 2.15 (3H, s), 2.10 (1H, m), 1.35 (9H, s) and 1.10-1.20 (6H, dd). Carboxylic acid proton exchanges with solvent.

### Example 57

### rel-(2S,4R,5R)-2-Isobutyl-1-(3-methoxy-4-tert-butylbenzoyl)-4-(5-methyl-1,2,4-oxadiazol-3-yl)-5-(1,3-thiazol-4-yl)pyrrolidine-2-carboxylic acid

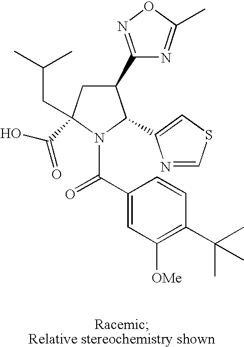

rel-(2S,4R,5R)-2-Isobutyl-4-(5-methyl-1,2,4-oxadiazol-3-yl)-5-(1,3-thiazol-4-yl)pyrrolidine-2-carboxylic acid, tert-butyl ester (Intermediate 50) was acylated with 3-methoxy-4-tert-butylbenzoyl chloride and subsequently subjected to ester hydrolysis with trifluoroacetic acid in a similar manner to that described for Example 27. The resulting oil was tritutated with diethyl ether to afford the title compound, a solid.

MS calcd for (C₂₇H₃₄N₄O₅S+H)⁺: 527. Found: (M+H)⁺=527.

¹H NMR (CD₃OD): δ 8.60 (1H, s), 7.40 (1H, s), 7.15 (1H, d), 6.90 (1H, d), 6.60 (1H, s), 5.65 (1H, d), 3.90-4.00 (1H, m), 3.75 (3H, s), 2.80 (1H, t), 2.65-2.70 (1H, dd), 2.60 (3H, s), 2.50 (1H, m), 2.10-2.15 (1H, dd), 1.90-2.00 (1H, m), 1.30 (9H, s), 1.20 (3H, d) and 1.05 (3H, d). Carboxylic acid proton exchanges with solvent.

### Example 58

### rel-(2S,4R,5R)-1-(4-tert-Butylbenzoyl)-4-(1,2,4-thiadiazol-5-yl)-2-isobutyl-5-(1,3-thiazol-2-yl)pyrrolidine-2-carboxylic acid

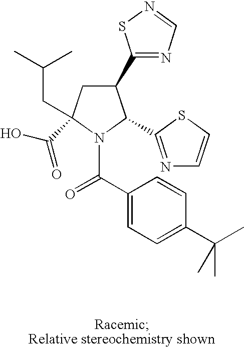

A mixture of rel-(2S,4S,5R)-1-(4-tert-butylbenzoyl)-1-[3-bromo-1,2,4-thiadiazol-5-yl]-2-isobutyl-5-(1,3-thiazol-2-yl)pyrrolidine-2-carboxylic acid (Example 48; 0.38 g, 0.66 mmol) ammonium formate (1.26 g, 0.02 mol), 10% palladium on carbon (90 mg) and ethanol (30 mL) containing 3 drops of water was heated at reflux for 1.5 hours. Additional portions of both palladium on carbon (0.11 g) and ammonium formate (1.3 g, 0.02 mol) were added after 1.5 hours and again after 4 hours and 6 hours and reflux continued for a total of 8 hours. The mixture was cooled and filtered through diatomaceous earth then concentrated to afford a solid. This solid was partitioned between ethyl acetate and water and the resulting ethyl acetate solution dried over Na₂SO₄ and evaporated. The resulting crude product was purified firstly by chromatography on silica gel using a gradient elution from ethyl acetate-cyclohexane (2:1 v/v) to ethyl acetate as eluent and then subsequently by reverse phase preparative HPLC on a C₁₈ column using a two-solvent gradient elution with (A) water containing formic acid (0.1%) and (B) acetonitrile-water (95:5 v/v) containing formic acid (0.05%) as eluents. The title compound, a solid, was confirmed by nOe NMR experiments to be inverted at the pyrrolidine C(4) centre relative to the starting material.

MS calcd for (C₂₅H₃₀N₄O₃S₂+H)⁺: 499. Found: (M+H)+=499.

¹H NMR (CD₃OD): δ 8.73 (1H, s), 7.39 (1H, d), 7.10-7.31 (5H, m), 5.79 (1H, d), 4.54 (1H, m), 2.97 (1H, t), 2.77 (1H, dd), 2.51-2.63 (1H, br m), 2.06 (1H, dd), 1.97 (1H, m), 1.24 (9H, s), 1.18 (3H, d) and 1.01 (3H, d). Carboxylic acid proton exchanges with solvent.

### Example 59

### rel-(2S,4R,5R)-2-Isobutyl-1-(3-methoxy-4-tert-butylbenzoyl)-4-(5-methyl-1,3,4-oxadiazol-2-yl)-5-(1,3-thiazol-2-yl)pyrrolidine-2carboxylic acid

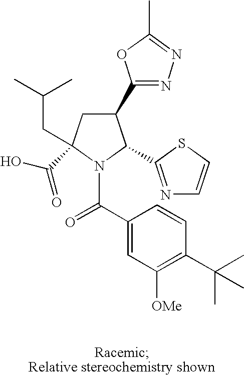

Stage A: A mixture of rel-(2S,4S,5R)- and rel-(2S,4R,5R)-2-Isobutyl-1-(3-methoxy-4-tert-butylbenzoyl)-4-hydrazinocarbonyl-5-(1,3-thiazol-2-yl)pyrrolidine-2-carboxylic acid, tert-butyl ester (Intermediate 52; 2.80 g, 5.01 mmol) and triethyl orthoacetate (80 mL) was heated under reflux for 6 days, then evaporated to a gum, a mixture of the rel-(2S,4S,5R)- and rel-(2S,4R,5R)-diastereoisomers of the title compound, tert-butyl ester.

MS calcd for (C₃₁H₄₂N₄O₅S+H)⁺: 583. Found: (M+H)⁺=583.

Stage B: The gum from Stage A (3.24 g, ca 5.56 mmol) was dissolved in trifluoroacetic acid (20 mL) and the solution stirred at room temperature for 5 hours, then evaporated. The residue was dissolved in dichloromethane (30 mL) and triethylamine (0.77 mL, 5.56 mmol) added. The mixture was evaporated and the resulting gum partitioned between ethyl acetate and water. The ethyl acetate solution was dried (Na₂SO₄) and evaporated and the crude product mixture purified by chromatography on silica gel using ethyl acetate-cyclohexane (2:1 v/v) as eluent The early eluting fractions were combined and evaporated and the resulting gum crystallised from diethyl ether to afford the title compound, shown by nOe NMR experiments to be the rel-(2S,4R,5R)-diastereoisomer.

MS calcd for (C₂₇H₃₄N₄O₅S+H)⁺: 527. Found: (M+H)⁺=527.

¹H NMR (CD₃OD): δ 7.44 (1H, d), 7.25 (1H, br s), 7.15 (1H, d), 6.92 (1H, br s), 6.91 (1H, d), 5.78 (1H, d), 4.17 (1H, m), 3.72 (3H, s), 2.88 (1H, t), 2.74 (1H, dd), 2.49 (1H, br), 2.46 (3H, s), 2.08 (1H, dd), 1.93 (1H, m), 1.29 (9H, s), 1.16 (3H, d), 1.01 (3H, d). Carboxylic acid proton exchanges with solvent.

Continued elution of the chromatography column afforded the corresponding rel-(2S,4S,5R)-diastereoisomer, described as Example 60 (Below).

### Example 60

### rel-(2 S,4 S,5R)-2-Isobutyl-1-(3-methoxy-4-tert-butylbenzoyl)-4-(5-methyl-1,3,4-oxadiazol-2-yl)-5-(1,3-thiazol-2-yl)pyrrolidine2-carboxylic acid

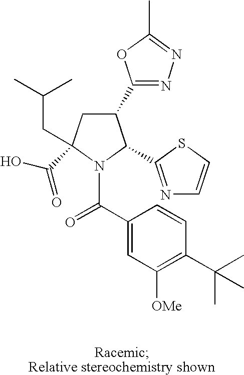

Continued elution of the chromatography column described in Example 59 (above) and subsequent evaporation of the later eluting fractions afforded a gum which was crystallised from diethyl ether-ethyl acetate (10:1 v/v) to afford the title compound, a solid.

MS calcd for (C₂₇H₃₄N₄O₅S+H)⁺: 527. Found: (M+H)⁺=527.

¹H NMR (CD₃OD): δ 7.69 (1H, d), 7.48 (1H, d), 7.20 (1H, d), 6.77 (1H, d), 6.44 (1H, s), 6.04 (1H, d), 4.57 (1H, m), 3.62 (3H, s), 3.05 (1H, t), 2.68 (1H, dd), 2.33 (3H, s), 2.31 (2H, d), 2.08 (1H, m), 1.28 (9H, s), 1.16 (3H, d) and 1.13 (3H, d). Carboxylic acid proton exchanges with solvent.

### Example 61

### rel-(2S,4S,5R)-2-Isobutyl-1-(4-tert-butylbenzoyl)-4-(5-methyl-1,3,4-oxadiazol-2-yl)-5-(1,3-thiazol-2-yl)pyrrolidine-2-carboxylic acid

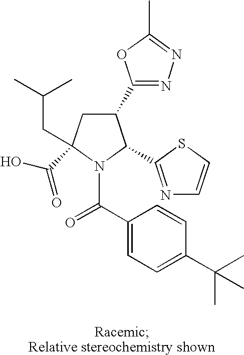

The title compound was prepared from rel-(2S,4S,5R)-2-Isobutyl-1-(4-tert-butylbenzoyl)-5-(1,3-thiazol-2-yl)-pyrrolidine-2,4-dicarboxylic acid, 2-tert-butyl ester, methyl ester (Intermediate 13) by sequentially following procedures analogous to those described for Intermediate 52 and Example 59. Purification of the crude reaction mixture by repeated chromatography on silica gel using ethyl acetate afforded the title compound, a solid from the earlier eluting fractions.

MS calcd for (C₂₆H₃₂N₄O₄S+H)⁺: 497. Found (M+H)⁺=497.

¹H NMR (CD₃OD): δ 7.62 (1H, d), 7.43 (1H, d), 7.29 (2H, d), 7.07 (2H, d), 6.06 (1H, d), 4.57 (1H, m), 3.05 (1H, t), 2.66 (1H, dd), 2.33 (3H, s), 2.24-2.38 (2H, m), 2.09 (1H, m), 1.24 (9H, s), 1.16 (3H, d) and 1.12 (3H, d). Carboxylic acid proton exchanges with the solvent.

The later eluting fractions from the chromatography on silica gel were combined to afford the corresponding rel-(2S,4R,5R)-diastereoisomer, described as Example 62 (below).

### Example 62

### rel-(2S,4R,5R)-2-Isobutyl-1-(4-tert-butylbenzoyl)-4-(5-methyl-1,3,4-oxadiazol-2-yl)-5-(1,3-thiazol-2-yl)pyrrolidine-2-carboxylic acid

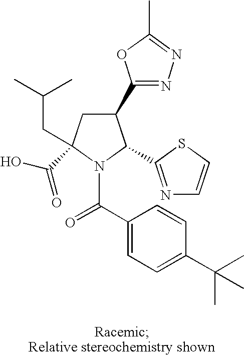

Continued elution of the chromatography column described in Example 61, (above) and subsequent evaporation of the later eluting fractions afforded the title compound, a solid.

MS calcd for (C₂₆H₃₂N₄O₄S+H)⁺: 497. Found (M+H)⁺=497.

¹H NMR (CD₃OD): δ 7.39 (1H, d), 7.25 (2H, d), 7.20 (2H, d), 7.19 (1H, br s), 5.78 (1H, d), 4.165 (1H, m), 2.88 (1H, t), 2.72 (1H, dd), 2.54 (1H, br d), 2.47 (3H, s), 2.06 (1H, dd), 1.93 (1H, m), 1.25 (9H, s), 1.16 (3H, d) and 1.00 (3H, d). Carboxylic acid proton exchanges with solvent.

### Example 63

### rel-(2S,4R,5R)-4-(5-Ethyl-1,2,4-oxadiazol-3-yl)-2-isobutyl-1-(3-methoxy-4-tert-butylbenzoyl)-5-(1,3-thiazol-2-yl)pyrrolidine-2-carboxylic acid

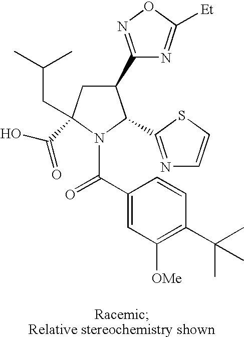

rel-(2S,4R,5R)-4-(5-ethyl-1,2,4-oxadiazol-3-yl)-2-isobutyl-5-(1,3-thiazol-2-yl)pyrrolidine-2-carboxylic acid, tert-butyl ester (Intermediate 53) was acylated with 3-methoxy-4-tert-butylbenzoyl chloride and subsequently subjected to ester hydrolysis with trifluoroacetic acid in a similar manner to that described for Example 27. The resulting oil was crystallised from diethyl ether to afford the title compound, a solid.

MS calcd for (C₂₈H₃₆N₄O₅S+H)⁺: 541. Found: (M+H)⁺=541.

¹H NMR (CD₃OD): δ 7.39 (1H, d), 7.18 (1H, br s), 7.14 (1H, d), 6.90 (1H, d), 6.60 (1H, br s), 5.74 (1H, d), 4.03 (1H, m), 3.71 (3H, s), 2.92 (2H, q), 2.85 (1H, t), 2.62 (1H, dd), 2.54 (1H, br d), 2.06 (1H, dd), 1.95 (1H, m), 1.35 (3H, t), 1.29 (9H, s), 1.17 (3H, d) and 1.02 (3H, d). Carboxylic acid proton exchanges with solvent.

### Example 64

### rel-(2S,4R,5R)-4-(5-Cyclopropyl-1,2,4-oxadiazol-3-yl)-2-isobutyl-1-(3-methoxy-4-tert-butylbenzoyl)-5-(1,3-thiazol-2-yl)pyrrolidine-2-carboxylic acid

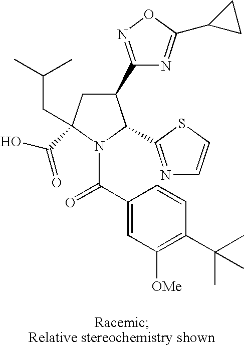

rel-(2S,4R,5R)-4-(5-cyclopropyl-1,2,4-oxadiazol-3-yl)-2-isobutyl-5-(1,3-thiazol-2-yl)pyrrolidine-2-carboxylic acid, tert-butyl ester (Intermediate 54) was acylated with 3-methoxy-4-tert-butylbenzoyl chloride and subsequently subjected to ester hydrolysis with trifluoroacetic acid in a similar manner to that described for Example 27. The resulting oil was crystallised from diethyl ether to afford the title compound, a solid.

MS calcd for (C₂₉H₃₆N₄O₅S+H)⁺: 553. Found: (M+H)⁺=553.

¹H NMR (CD₃OD): δ 7.39 (1H, d), 7.18 (1H, br), 7.14 (1H, d), 6.90 (1H, d), 6.58 (1H, br), 5.71 (1H, d), 3.98 (1H, m), 3.70 (3H, s), 2.81 (1H, t), 2.59 (1H, dd), 2.51 (1H, br d), 2.24 (1H, m), 2.05 (1H, dd), 1.93 (1H, m), 1.29 (9H, s), 1.11-1.29 (4H, m), 1.16 (3H, d) and 1.01 (3H, d). Carboxylic acid proton exchanges with solvent.

### Example 65

### rel-(2S,4S,5R)-2-Isobutyl-1-(3-methoxy-4-tert-butylbenzoyl)-4-(5-methyl-1,2,4-thiadiazol-3-yl)-5-(1,3-thiazol-2-yl)pyrrolidine-2-carboxylic acid

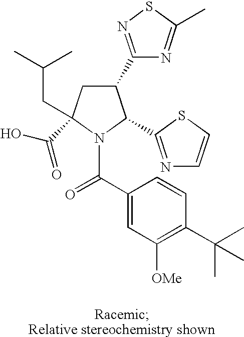

Stage A: 3-Methoxy-4-tert-butylbenzoyl chloride (0.13 g, 0.58 mmol) was added to a stirred solution of rel-(2S,4S,5R)-2-isobutyl-4-(5-methyl-1,2,4-thiadiazol-3-yl)-5-(1,3-thiazol-2-yl)pyrrolidine-2-carboxylic acid, tert butyl ester (Intermediate 57; 0.20 g, 0.49 mmol) and triethylamine (0.083 mL, 0.60 mmol) in anhydrous dichloromethane (10 mL). The mixture was stirred for 42 hours at room temperature, then diluted with dichloromethane and washed with saturated aqueous sodium bicarbonate solution. The dichloromethane solution was evaporated and the residue purified by chromatography on silica gel using cyclohexane-ethyl acetate (3:1 v/v) as eluent to afford the title compound tert-butyl ester, a foam.

MS calcd for (C₃₁H₄₂N₄O₄S₂+H)⁺: 599. Found: (M+H)⁺=599.

Stage B: The tert-butyl ester (0.22 g, 0.37 mmol) was dissolved in trifluoroacetic acid (6 mL), stirred at room temperature for 4 hours then evaporated. The residue was re-evaporated twice from dichloromethane and finally crystallised from diethyl ether to afford the title compound, a solid.

MS calcd for (C₂₇H₃₄N₄O₄S₂+H)⁺: 543. Found: (M+H)⁺=543.

¹H NMR (CD₃OD): δ 7.69 (1H, d), 7.37 (1H, d), 7.19 (1H, d), 6.75 (1H, br d), 6.42 (1H, br s), 5.98 (1H, d), 4.63 (1H, m), 3.62 (3H, s), 3.21 (1H, t), 2.64(3H, s), 2.62 (1H, dd), 2.33 (2H, d), 2.11 (1H, m), 1.27 (9H, s), 1.18 (3H, d) and 1.13 (3H, d). Carboxylic acid proton exchanges with solvent.

### Example 66

### rel-(2S,4S,5R)-1-(6-tert-Butylbenzoyl)-2-isobutyl-4-(5-methyl-1,2,4-thiadiazol-3yl)-5-(1,3-thiazol-2-yl)pyrrolidine-2-carboxylic acid

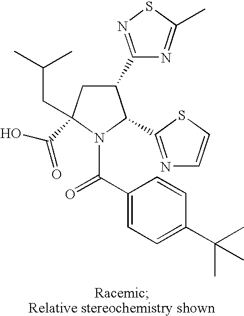

rel-(2S,4S,5R)-2-Isobutyl-4-(5-methyl-1,2,4-thiadiazol-3-yl)-5-(1,3-thiazol-2-yl)pyrrolidine-2-carboxylic acid, tert butyl ester (Intermediate 57) was acylated with 4-tert-butylbenzoyl chloride and subsequently subjected to ester hydrolysis with trifluoroacetic acid in a similar manner to that described for Example 65. The resulting oil was tritutated with diethyl ether to afford the title compound, a solid.

MS calcd for (C₂₆H₃₂N₄O₃S₂+H)⁺: 513. Found: (M+H)⁺=513.

¹H NMR DMSO-d₆): δ 14.11 (1H, br s), 7.65 (1H, d), 7.47 (1H, d), 7.24 (2H, d), 7.05 (2H, d), 5.96 (1H, d), 4.58 (1H, m), 2.99 (1H, t), 2.62 (3H, s), 2.55 (1H, d), 2.18 (2H, d), 2.05 (1H, m) 1.20 (9H, s) and 1.05 (6H,m).

### Example 67

### rel-(2S,4S,5R)-1-(3-Methoxy-4-tert-butylbenzoyl)-2-methyl-4-(3-methyl-1,2,4-oxadiazol-5-yl)-5-(1,3-thiazol-2-yl)pyrrolidine-2-carboxylic acid

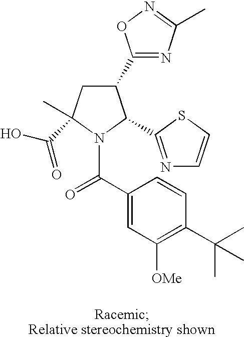

rel-(2S,4S,5R)-2-1-methyl-4-(3-methyl-1,2,4-oxadiazol-5-yl)-5-(1,3-thiazol-2-yl)pyrrolidine-2-carboxylic acid, tert-butyl ester (Intermediate 58) was acylated with 3-methoxy-4-tert-butylbenzoyl chloride and subsequently subjected to ester hydrolysis with trifluoroacetic acid in a similar manner to that described for Example 65. The resulting oil was tritutated with diethyl ether to afford the title compound, a solid.

MS calcd for (C₂₄H₂₈N₄O₅S+H)⁺: 485. Found (M+H)⁺=485.

¹H NMR (CDCl₃): δ 7.50 (1H, s), 7.43 (1H, s), 7.19 (1H, d), 6.90 (1H, d), 6.74 (1H, s), 6.07 (1H, d), 4.17 (1H, dd), 3.76 (3H, s), 2.92 (1H, dd), 2.67 (1H, dd), 2.37 (3H, s), 1.69 (3H, s), 1.31 (9H, s). The carboxylic acid proton exchanges with the solvent.

### Example 68

### Enantiomer A of rel-(2S,4R,5R)-1-(3-bromo-4-tert-butylbenzoyl)-4-(3-methyl-1,2,4-oxadiazol-5-yl)-2-isobutyl-5-pyridin-2-ylpyrrolidine-2-carboxylic acid

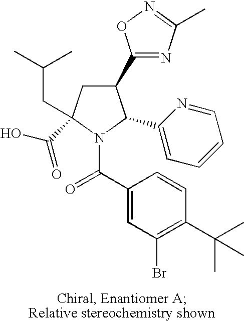

Stage A: A mixture of Enantiomer A of rel-(2S,4S,5R)-4-(aminocarbonyl)-1-(3-bromo-4-tert-butylbenzoyl)-2-isobutyl-5-pyridin-2-yl-pyrrolidine-2-carboxylic acid, tert-butyl ester (Intermediate 62; 0.26 g, 0.44 mmol) and (1,1-dimethoxyethyl)dimethylamine (12.5 mL) was heated at 120° C. for 2 hours then evaporated. The residue was dissolved in dioxan (3 mL) containing acetic acid (3 mL) and hydroxylamine hydrate (38 uL) was added. The mixture was heated at 90° C. for 3 hours then concentrated and partitioned between water (25 mL) and ethyl acetate (25 mL). The ethyl acetate solution was dried (MgSO₄) and evaporated to afford a gum which was purified by preparative TLC on silica gel using cyclohexane-ethyl acetate (2:1 v/v) as eluent to afford the title compound, a gum (0.064 g). This material was shown by nOe NMR studies to be the tert-butyl ester of the rel-(2S,4R,5R)-diastereoisomer title compound, epimerised at the pyrrolidine C(4) centre relative to the starting material.

MS calcd for (C₃₂H₄₁BrN₄O₄+H)⁺: 625/627 Found: (M+H)⁺=625/627.

¹H NMR (CDCl₃): δ 8.08 (1H, d), 7.73 (1H, d), 7.47 (1H, dt), 7.20 (1H, d), 7.15 (1H, s), 7.12 (1H, dd), 6.92 (1H, m), 5.36 (1H, d), 4.13 (1H, m), 2.78 (1H, t), 2.55 (1H, m), 2.53 (1H, dd), 2.34 (3H, s), 1.98 (1H, dd), 1.89 (1H, m), 1.61 (9H, s), 1.37 (9H, s), 1.15 (3H, d) and 1.02 (3H, d).

Continued elution of the preparative TLC plate afforded the rel-(2S,4S,5R)-diastereoisomer (0.079 g).

Stage B: —The tert-butyl ester of the rel-(2S,4R,5R)-diastereoisomer (Stage A above; 0.064 g, 0.102 mmol) was dissolved in dichloromethane (1 mL) and trifluoroacetic acid (1 mL) was added. The mixture was stirred at room temperarture overnight then concentrated. The residue was dissolved in THF (5 mL) and aqueous sodium hydroxide solution (0.1 M, 1.02 mL) was added. The mixture was stirred at room temperature for 2 hours then concentrated. The resulting material was suspended in water (10 mL) and the solid filtered off. This solid was re-evaporated from methanol (10 mL) to afford the title compound, a solid.

MS calcd for (C₂₈H₃₀BrN₄O₄+H)⁺: 569/571. Found: (M+H)⁺=569/571.

¹H NMR (CD3OD): δ 8.25 (1H, d), 7.59 (1H, m), 7.41 (1H, m), 7.32 (1H, d), 7.14-7.22 (3H, m), 5.51 (1H, d), 4.07 (1H, m), 2.91 (1H, t), 2.78 (1H, m), 2.50 (1H, m), 2.34 (3H, s), 2.12 (1H, m), 1.91 (1H, m), 1.42 (9H, s), 1.15 (3H, d) and 0.99 (3H, d). Carboxylic acid proton exchanges with solvent.

### Example 69

### rel-(2S,4R,5R)-2-Isobutyl-1-(3-methoxy-4-tert-butylbenzoyl)-4-(3-methyl-isoxaxol-5-(1,3-thiazol-2-yl)pyrrolidine-2-carboxylic acid

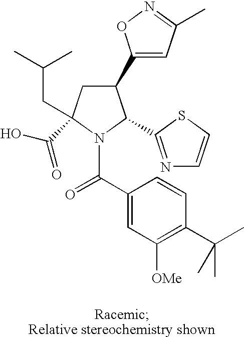

Stage A: rel-(2R, 4R,5R)-2-Isobutyl-4-(3-methyl-isoxaxol-5-yl)-5-(1,3-thiazol-2-yl)pyrrolidine-2-carboxylic acid, tert-butyl ester (Intermediate 63; 0.10 g, 0.26 mmol) and triethylamine (40 uL, 0.29 mmol) were added to a solution of 3-methoxy-4-tert-butylbenzoyl chloride (0.065 g, 0.29 mmol) in dry dichloromethane (1 mL) at room temperature under nitrogen. The mixture was stirred for 72 hours then diluted with dichloromethane (15 mL) and washed with water (15 mL). The dichloromethane solution was dried (MgSO₄) and evaporated to afford a gum, subsequently purified by chromatography on silica gel using a gradient of cyclohexane-ethyl acetate (from 95:5 v/v to 80:20 v/v) as eluent to afford the title compound, tert-butyl ester (0.059 g), a foam.

Stage B: The tert-butyl ester was dissolved in trifluoroacetic acid (2 mL) and stirred at room temperature for 1 day before being concentrated in vacuo. The residue was re-evaporated from dichloromethane (×2) then toluene, then triturated with diethyl ether to afford the title compound, a solid. nOe NMR spectroscopy showed this to be the rel-(2S,4R,5R)-diastereoisomer.

MS calcd for (C₂₈H₃₅N₃O₅S+H)⁺: 526. Found: (M+H)⁺=526.

¹H NMR (CD₃OD) δ 7.40 (1H, d), 7.09-7.20 (2H, m), 6.90 (1H, d), 6.55 (1H, brs), 6.09 (1H, s), 5.59 (1H, d), 4.04-4.13 (1H, m), 3.70 (3H, s), 2.50-2.80 (3H, m), 2.18 (3H, s), 1.87-2.09 (2H, m), 1.29 (9H, s), 1.16 (3H, d) and 1.02 (3H, d). Carboxylic acid proton exchanges with the solvent.

### Example 70

### Enantiomer A of rel-(2S,4R,5R)-2-Isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid

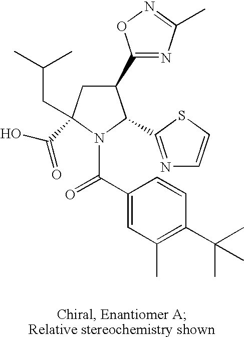

Stage A: Enantiomer A of rel-(2S,4R,5R)-2-isobutyl-4-3-methyl-1,2,4-oxadiazol-5-yl)-5-(1,3-thiazol-2-yl)pyrrolidine-2-carboxylic acid, tert-butyl ester (Intermediate 25; 0.170 g, 0.434 mmol) was dissolved in chloromethane (5 mL) and 3-methyl-4-tert-butylbenzoyl chloride (1.2 eq) and triethylamine (1.25 eq) were added. The mixture was stirred for 24 hours at room temperature, then concentrated and the residue purified by chromatography on silica gel using a gradient of cyclohexane-ethyl acetate (from 100:0 to 80:20 v/v) as eluent to afford the title compound tert-butyl ester, an oil.

Stage B: The tert-butyl ester (0.025 g, 0.044 mmol) was dissolved in trifluoroacetic acid (2 mL) and stirred for 3 hours at room temperature. The mixture was evaporated and the residue purified by chromatography on silica gel using a gradient of cyclohexane-ethyl acetate (from 100:0 to 50:50 v/v) as eluent to afford the title compound, a solid.

MS calcd for (C₂₇H₃₄N₄O₄S+H)⁺: 511. Found: (M+H)⁺=511.

¹H NMR (CD₃OD): δ 7.45 (1H, d), 7.25 (2H, m), 7.05 (1H, d), 6.90 (1H, s), 5.80 (1H, d), 4.15-4.25 (1H, m), 2.90 (1H, t), 2.75 (1H, dd), 2.5 (1H, br), 2.40 (3H, s), 2.35 (3H, s), 2.05 (1H, m), 1.95 (1H, m), 1.35 (9H, s), 1.15 (3H, d) and 1.00 (3H, d). Carboxylic acid proton exchanges with solvent.

### Example 71

### rel-(2S,4S,5R)-2-Isobutyl-1-(3-methoxy-4-tert-butylbenzoyl)-4-(3-methyl-1,2,4-oxadiazol-5-yl)-5-(1,3-thiazol-4-yl-pyrrolidine-2-carboxylic acid

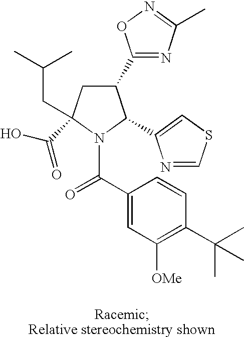

Stage A: 3-methoxy-4-tert-butylbenzoyl chloride (0.173 g, 0.765 mmol) and triethylamine (0.11 mL, 0.797 mmol) were added to a solution of rel-(2S,4S,5R)-2-Isobutyl-4-(3-methyl-1,2,4-oxadiazol-5-yl)-5-(1,3-thiazol-4-yl)pyrrolidine-2-carboxylic acid, tert-butyl ester (Intermediate 65; 0.25 g, 0.638 mmol) in dichloromethane (5 mL). The mixture was stirred at RT for 18 hours then evaporated. The residue was purified by chromatography on silica gel using a gradient of cyclohexane-ethyl acetate (from 100:0 to 80:20 v/v) as eluent to afford the title compound tert-butyl ester, a foam.

Stage B: The tert-butyl ester (0.27 g, 0.464 mmol) was dissolved in trifluoroacetic acid (3 mL) and stirred at room temperature for 4 hours, then evaporated. The residue was triturated with diethyl ether to afford the title compound, a solid.

MS calcd for (C₂₇H₃₄N₄O₅S+H)⁺: 527. Found: (M+H)⁺=527.

¹H NMR (CD₃OD): δ 8.90 (1H, s), 7.30 (1H, s), 7.20 (1H, d), 6.80 (1H, d), 6.45 (1H, s), 5.90 (1H, d), 4.60 (1H, m), 3.65 (3H, s), 3.05 (1H, t), 2.70 (1H, dd), 2.35 (2H, d), 2.15 (3H, s), 2.10 (1H, m), 1.30 (9H, s), 1.20 (3H, d) and 1.15 (3H, d). Carboxylic acid proton exchanges with solvent.

### Example 72

### rel-(2S,4R,5R)-2-Isobutyl-1-(3-methoxy-4-tert-butylbenzoyl)-4-(3-methyl-1,2,4-oxadiazol-5-yl)-5-(1,3-thiazol-4-yl)pyrrolidine-2-carboxylic acid

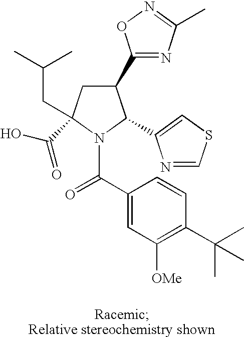

Stage A: 3-methoxy-4-tert-butylbenzoyl chloride (0.194 g, 0.857 mmol) and triethylamine (0.12 mL, 0.893 mmol) were added to a solution of rel-(2S,4R,5R-2-Isobutyl-4-3-methyl-1,2,4-oxadiazol-5-yl)-5-(1,3-thiazol-4-yl)pyrrolidine-2-carboxylic acid, tert-butyl ester (Intermediate 66; 0.28 g, 0.714 mmol) in dichloromethane (5 mL). The mixture was stirred at RT for 3 hours then evaporated. The residue was purified by chromatography on silica gel using a gradient of cyclohexane-ethyl acetate (from 100:0 to 80:20 v/v) as eluent to afford the title compound tert-butyl ester, a foam.

Stage B: The tert-butyl ester (0.29 g, 0.498 mmol) was dissolved in trifluoroacetic acid (3 mL) and stirred at room temperature for 4 hours, then evaporated. The residue was triturated with diethyl ether to afford the title compound, a solid.

MS calcd for (C₂₇H₃₄N₄O₅S+H)⁺: 527. Found: (M+H)⁺=527.

¹H NMR (CD₃OD): δ 8.60 (1H, s), 7.40 (1H, s), 7.15 (1H, d), 6.90 (1H, d), 6.60 (1H, s), 5.70 (1H, d), 4.10-4.20 (1H, m), 3.75 (3H, s), 2.85 (2H, m), 2.50 (1H, dd), 2.35 (3H, s), 2.10-2.20 (1H, dd), 1.90-2.00 (1H, m), 1.30 (9H, s), 1.20 (3H, d) and 1.05 (3H, d). Carboxylic acid proton exchanges with solvent.

The compounds according to the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

The compounds of the present invention can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula(I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxological effects are expected when compounds of the present invention are administered in accordance with the present invention.

### Assay

The potential for compounds of the invention to inhibit NS5B wildtype HCV polymerase activity may be demonstrated, for example, using the following in vitro assay:

In Vitro Detection of Inhibitors of HCV RNA-Dependent RNA Polymerase Activity

Incorporation of [³H]-UMP into RNA was followed by absorption of the RNA polymer onto a DEAE glass fibre fiber. A synthetic template consisting of 16 mer oligoU hybridised to polyrA (10:1 w/w) was used as a homopolymer substrate.

Reaction Conditions were 22 μM [H]-UTP (0.75 Ci/mMol), 1 mM-Dithiothreitol, 3.2 mM-MgCl₂, 20 mM-Tris-HCl, pH7.0, 10 μg/mL polyA-oligoU, and 90 mM-NaCl. Note that 50 mM-NaCl is added with the enzyme

HCV RNA Polymerase (Recombinant full-length NS5B (Lohmann et al, J. Virol. 71 (11), 1997, 8416 ‘Biochemical properties of hepatitis C virus NS5B RNA-dependent RNA polymerase and identification of amino acid sequence motifs essential for enzymatic activity’) expressed in baculovirus and purified to homogeneity) was diluted to about 50 μg protein/mL (dependent on specific activity) in 50 mM-Hepes, pH7.0, 0.5M-NaCl, 20%-Glycerol, 0.05%-Triton X-100, 5 mM-Dithiothreitol, 0.1 mM-EDTA.

5× Concentrated Buffer mix was prepared using 1M-Tris-HCl (pH7.0, 1 mL), 1M-MgCl₂ (0.16 mL), 1M-Dithiothreitol (0.05 mL), 5M-NaCl (0.4 mL), and Water (8.4 mL), Total 10 mL.

Substrate Mix was prepared using 5× Concentrated Buffer mix (12 μL), [³H]-UTP (1 μCi/μL; 21.7 μM, 1 μL), 22 μM-UTP (100 μM, 13.2 μL), 10 μg/mL polyA-oligoU (100 μg/mL, 6μL), and Water (12.8 μL), Total 45 μL.

The Assay was set up using Substrate Mix (45 μL), compound (10 μL), and Diluted Enzyme (added last to start reaction) (5 μL), Total 60 μL.

The reaction was performed in a U-bottomed, clear, 96-well plate. The reaction was mixed on a plate-shaker, after addition of the Enzyme, and incubated for 2 h at 22° C. After this time, the reaction was stopped by addition of 25 μL of 100 mM-EDTA.

A DEAE Filtermat (Part No. 1205-405 from Pharmacia) was pre-washed in water and alcohol and dried. 2×20 μL of the Stopped Assay Mix was spotted onto a square of the DEAE Filtermat. The DEAE Filtermat was washed for 2×15 min in SSC buffer (0.3M-NaCl, 30 mM-Na Citrate) followed by 2×2 min in water and 1×1 min in alcohol. The Filtermat was dried and sealed in a bag together with 10 mL of OptiScint HiSafe scintillation fluid. The radioactivity present on the filtermat was detected by scintillation counting on a Wallac 1205 Betaplate counter. After subtraction of background levels without enzyme, any reduction in the amount of radioactivity incorporated in the presence of a compound, compared to that in the absence, was taken as a measure of the level of inhibition. Ten concentrations of compounds were tested in two- or threefold dilutions. From the counts, percentage of inhibition at highest concentration tested or IC₅₀s for the compounds were calculated using Grafit3 or Grafit4 software packages.

The exemplified compounds all had an IC₅₀ of <50 μM. Accordingly, the compounds of the invention are of potential therapeutic benefit in the treatment and prophylaxis of HCV. Preferred compounds had an IC₅₀ of <1 μM.

Thus, there is provided as a further aspect of the present invention a compound of formula (I) or a physiologically acceptable salt or solvate thereof for use in human or veterinary medicine, particularly in the treatment or prophylaxis of viral infection, particularly HCV infection.

It will be appreciated that reference herein to treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of a compound of formula (I) or a physiologically acceptable salt or solvate thereof for the manufacture of a medicament for the treatment and/or prophylaxis of viral infection, particularly HCV infection.

According to another aspect of the invention, there is provided a compound of formula (I) or a physiologically acceptable salt or solvate thereof for use in medical therapy; particularly use in the treatment and/or prophylaxis of a viral infection, particularly HCV infection.

In a further or alternative aspect there is provided a method for the treatment of a human or animal subject with viral infection, particularly HCV infection, which method comprises administering to said human or animal subject an effective amount of a compound of formula (I) or a physiologically acceptable salt or solvate thereof.

The pharmaceutical compositions according to the invention may also be used in combination with other therapeutic agents, for example immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

The invention thus provides, in a further aspect, a combination comprising a compound of formula (I) or a physiologically acceptable salt or solvate thereof together with another therapeutically active agent.

The combinations referred to above may conveniently be presented for use in the form of a pharmaceutical formulation and thus pharmaceutical formulations comprising a combination as defined above together with a pharmaceutically acceptable carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

All publications, including but not limited to patents and patent applications cited in this specification are herein incorporated by reference as if each individual publication were specifically and individually indicated to be incorporated by reference as though fully set forth.

1. A compound of formula (I) 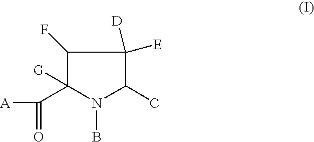
wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or R¹ and R² together with the nitrogen atom to which they are attached form a 5 or 6 membered saturated cyclic group;
B represents C(O)R³ wherein R³ is selected from the group consisting of aryl and heteroaryl;
C represents C₁₋₆alkyl, aryl, heteroaryl or heterocyclyl;
D represents a saturated or unsaturated 5-membered heterocyclic ring comprising one or more carbon atoms, each of which may independently be optionally substituted by R⁴ and R⁵, and one to four heteroatoms independently selected from N, optionally substituted by hydrogen, C₁₋₆alkyl, C(O)R³, SO₂R³, aryl, heteroaryl, arylalkyl, or heteroarylalkyl; O; and S, optionally substituted by one or two oxygen atoms; wherein the 5 membered ring may be attached at any endocyclic carbon atom, and may be optionally fused via two adjacent carbon atoms to a saturated or unsaturated 6 membered carbocyclic or heterocyclic ring which may itself be optionally substituted on a non-fused carbon atom by C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl;
R⁴ and R⁵ are independently selected from hydrogen, C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl;
R⁶ and R⁷ are independently selected from hydrogen, C₁₋₆alkyl, aryl and heteroaryl; and
R⁸ represents hydrogen, C₁₋₆alkyl, arylalkyl, or heteroarylalkyl;
E represents hydrogen or C₁₋₆alkyl;
F represents hydrogen, C₁₋₆alkyl, aryl or heteroaryl; and
G represents hydrogen, C₁₋₆alkyl, heterocyclylalkyl, arylalkyl or heteroarylalkyl; and salts, solvates and esters thereof, provided that when A is OR¹ then R¹ is other than tert-butyl.

2. Compounds of Formula (I) as claimed in claim 1 in which B represents C(O)R³, and R³ represents phenyl substituted in the para-position by tert-butyl and optionally further substituted by methyl, ethyl, methoxy, ethoxy, or halo.

3. Compounds of Formula (I) as claimed in claim 1, in which B represents C(O)R³, and R³ represents phenyl substituted in the para-position by tert-butyl and optionally further substituted in the meta-position by methyl, ethyl, methoxy, ethoxy, or halo.

4. Compounds of Formula (I) as claimed in claim 1, in which C is selected from the group consisting of C₁₋₆alkyl, aryl and heteroaryl.

5. Compounds of Formula (I) as claimed in claim 1, in which D is selected from the group (i) consisting of 1H-pyrrol-2-yl, 1H-pyrrol-3-yl, furan-2-yl, furan-3-yl, thien-2-yl, thien-3-yl; 1H-imidazol-2-yl, 1H-pyrazol-3-yl, 1H-pyrazol-5-yl, isoxazol-3-yl, isoxazol-5-yl, oxazol-2-yl, isothiazol-3-yl, isothiazol-5-yl, thiazol-2-yl, 1,3-dioxol-2-yl, 1,3-oxathiazol-2-yl, and 1,3-dithiol-2-yl, and partially or fully saturated derivatives thereof; each of which, where applicable, may be optionally substituted on a carbon atom by R⁴ and R⁵, on a nitrogen atom by hydrogen, C₁₋₆alkyl, C(O)R³, SO₂R³, aryl, heteroaryl, arylalkyl, or heteroarylalkyl, and on a sulphur atom by one or two oxygen atoms; and each of which may be optionally fused via two adjacent ring carbon atoms to a saturated or unsaturated 6 membered carbocyclic or heterocyclic ring which may itself be optionally substituted on a non-fused carbon atom by C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl;
or the group (ii) consisting of 1H-imidazol-4-yl, 1H-imidazol-5-yl, 1H-pyrazol-4-yl, isoxazol-4-yl, oxazol-4-yl, oxazol-5-yl, isothiazol-4-yl, thiazol-4-yl, thiazol-5-yl, 1,3-dioxol-4-yl, 1,3-oxathiazol-4-yl, 1,3-oxathiazol-5-yl, 1,3-dithiol-4-yl, 1H-1,2,3-triazol-4-yl, 1H-1,2,3-triazol-5-yl, 2H-1,2,3-triazol-4-yl, 1H-1,2,4-triazol-3-yl, 1H-1,2,4-triazol-5-yl, 4H-1,2,4-triazol-3-yl, 1,2,4-oxadiazol-5-yl, 1,2,4-oxadiazol-3-yl, 1,2,5-oxadiazol-3-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl, 1,3,4-thiadiazol-2-yl, 1,3,2-oxathiazol-4-yl, 1,3,2-oxathiazol-5-yl, 1,3,4-oxathiazol-2-yl, 1,3,4-oxathiazol-5-yl, 1H-tetrazol-5-yl, and 2H-tetrazol-5-yl, and partially or fully saturated derivatives thereof; each of which, where applicable, may be optionally substituted on a carbon atom by R⁴ and/or R⁵, on a nitrogen atom by hydrogen, C₁₋₆alkyl, C(O)R³, SO₂R³, aryl, heteroaryl, arylalkyl, or heteroarylalkyl, and on a sulphur atom by one or two oxygen atoms.

6. Compounds of Formula (I) as claimed claim 1, in which D is selected from the group consisting of 5-methyl-1,2,4-thiadiazol-3-yl; 1,2,4-thiadiazol-5-yl; 3-bromo-1,2,4-thiadiazol-5-yl; 3-methyl-1,2,4-oxadiazol-5-yl; 5-methyl-1,2,4-oxadiazol-3-yl; 5-methyl-1,3,4-oxadiazol-2-yl; 5-ethyl-1,2,4-oxadiazol-3-yl; 5-cyclopropyl-1,2,4-oxadiazol-3-yl; 3-methyl-isoxaxol-5-yl; 1H-1,2,4-triazol-3-yl; 5-methyl-1H-1,2,4-triazol-3-yl; 1,2,4-oxadiazol-5-yl; 3-(4-fluorophenyl)-1,2,4-oxadiazol-5-yl; 1,2,4-oxadiazol-3-yl; 5(4H)-1,2,4-oxadiazolon-3-yl; 1,3,4-oxadiazol-2-yl; 1,3,4-thiadiazol-2-yl; isoxazol-5-yl; 3-methyl-isoxazol-5-yl; 3-methyl-pyrazol-5-yl; thiazol-2-yl; 1-methyl-1H-tetrazol-5-yl; benzothiazol-2-yl; and benzoxazol-2-yl.

7. Compounds of Formula (I) as claimed in claim 1, in which G is isobutyl, benzyl or methyl.

8. A compound as claimed in claim 1 selected from the group consisting of:
rel-(2S,4R,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid;
rel-(2S,4S,5R)-1-(4-tert-butylbenzoyl)-2-isobutyl-4-(5-methyl-1H-1,2,4-triazol-3-yl)-5-(1,3-thiazol-2-yl)pyrrolidine-2-carboxylic acid;
rel-(2S,4S,5R)-1-(4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4S,5R)-1-(4-tert-butylbenzoyl)-2-isobutyl-5-(1,3-thiazol-2-yl)-4-(1H-1,2,4-triazol-3-yl)pyrrolidine-2-carboxylic acid;
rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-(1,2,4-oxadiazol-5-yl)-5-(1,3-thiazol-2-yl)pyrrolidine-2-carboxylic acid; and
rel-(2S,4R,5R)-1-(4-tert-butylbenzoyl)-2-isobutyl-4-(5-methyl-1H-1,2,4-triazol-3-yl)-5-thien-2-yl-pyrrolidine-2-carboxylic acid;
rel-(2S,4R,5R)-1-(4-tert-butylbenzoyl)-2-isobutyl-4-(3-methyl-1,2,4-oxadiazol-5-yl)-5-thien-2-ylpyrrolidine-2-carboxylic acid;
rel-(2S,4S,5R)-1-(4-tert-butylbenzoyl)-2-isobutyl-4-(5-methyl-1,2,4-oxadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(1,2,4-oxadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxamide;
rel-(2S,4S,5R)-1-(3-bromo-4-tert-butylbenzoyl)-2-isobutyl-4-(1,2,4-oxadiazol-5-yl)-5-(1,3-thiazol-2-yl)pyrrolidine-2-carboxylic acid;
rel-(2S,4R,5R)-1-(4-tert-butylbenzoyl)-2-isobutyl-4-[5(4H)-1,2,4-oxadiazolon-3-yl)-5-(1,3-thiazol-2-yl)pyrrolidine-2-carboxylic acid;
rel-(2S,4R,5R)-1-(4-tert-butylbenzoyl)-2-isobutyl-4-(isoxazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methylisoxazol-5-yl)-5-(1,3-thiazol-2-yl)pyrrolidine-2-carboxylic acid;
rel-(2S,4R,5R)-1-(4-tert-butylbenzoyl)-2-isobutyl-4-(3-methylpyrazol-5-yl)-5-(1,3-thiazol-2-yl)pyrrolidine-2-carboxylic acid;
rel-(2S,4R,5R)-1-(4-tert-butylbenzoyl)-2-isobutyl-4-(1,3-thiazol-2-yl)-5-(1,3-thiazol-2-yl)pyrrolidine-2-carboxylic acid;
rel-(2S,4S,5R)-1-(4-tert-butylbenzoyl)-2-isobutyl-4-(1,3-thiazol-2-yl)-5-(1,3-thiazol-2-yl)pyrrolidine-2-carboxylic acid;
rel-(2S,4R,5R)-2-isobutyl-1-(4-tert-butylbenzoyl)-4-(1,3,4-oxadiazol-2-yl)-5-(1,3-thiazol-2-yl)-pyrrolidine-2-carboxylic acid;
rel-(2S,4S,5R)-2-isobutyl-1-(4-tert-butylbenzoyl)-4-(1,3,4-oxadiazol-2-yl)-5-(1,3-thiazol-2-yl)-pyrrolidine-2-carboxylic acid;
rel-(2S,4R,5R)-2-isobutyl-1-(4-tert-butylbenzoyl)-4-(1,3,4-thiadiazol-2-yl)-5-(1,3-thiazol-2-yl)-pyrrolidine-2-carboxylic acid;
rel-(2R,4S,5R)-2-benzyl-1-(4-tert-butylbenzoyl)-4-(1,2,4-oxadiazol-5-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(1-methyl-1H-tetrazol-5-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(benzothiazol-2-yl)-5-(1,3-thiazol-2-yl)-pyrrolidine-2-carboxylic acid;
rel-(2S,4R,5R)-2-isobutyl-1-(3-bromo-4-tert-butylbenzoyl)-4-(benzothiazol-2-yl)-5-(1,3-thiazol-2-yl)-pyrrolidine-2-carboxylic acid;
rel-(2S,4S,5R)-2-isobutyl-1-(3-bromo-4-tert-butylbenzoyl)-4-(benzoxazol-2-yl)-5-(1,3-thiazol-2-yl)-pyrrolidine-2-carboxylic acid;
rel-(2S,4R,5R)-2-isobutyl-1-(3-bromo-4-tert-butylbenzoyl)-4-(benzoxazol-2-yl)-5-(1,3-thiazol-2-yl)-pyrrolidine-2-carboxylic acid;
rel-(2S,4S,5R)-1-(4-tert-butylbenzoyl)-2-isobutyl-4-(1,2,4-oxadiazol-5-yl)-5-pyridin-2-yl-pyrrolidine-2-carboxylic acid;
rel-(2S,4R,5R)-1-(4-tert-butylbenzoyl)-2-isobutyl-4-(1,2,4-oxadiazol-5-yl)-5-pyridin-2-yl-pyrrolidine-2-carboxylic acid;
rel-(2S,4S,5R)-1-(4-tert-butylbenzoyl)-2-isobutyl-4-(1,2,4-oxadiazol-5-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxylic acid;
rel-(2S,4S,5R)-1-(3-chloro-4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-1-(3-chloro-4-tert-butylbenzoyl)-2-isobutyl-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-2-yl)pyrrolidine-2-carboxamide;
rel-(2S,4R,5R)-2-isobutyl-1-(3-methoxy-4-tert-butylbenzoyl)-4-(5-methyl-1,2,4-oxadiazol-3-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-oxadiazol-3-yl)-5-(1,3-thiazol-2-yl)pyrrolidine-2-carboxylic acid;
rel-(2S,4S,5R)-5-(benzothiazol-2-yl)-2-isobutyl-1-(4-tert-butylbenzoyl)-4-(1,2,4-oxadiazol-5-yl)-pyrrolidine-2-carboxylic acid;
rel-(2S,4S,5R)-5-(benzothiazol-2-yl)-2-isobutyl-1-(3-bromo-4-tert-butyl-benzoyl)-4-(1,2,4-oxadiazol-5-yl)-pyrrolidine-2-carboxylic acid;
rel-(2S,4S,5R)-1-(3-bromo-4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid;
rel-(2S,4S,5R)-1-(4-tert-butylbenzoyl)-4-[3-(4-fluorophenyl)-1,2,4-oxadiazol-5-yl]-2-isobutyl-5-(1,3-thiazol-2-yl)pyrrolidine-2-carboxylic acid;
rel-(2S,4R,5R)-1-(4-tert-butylbenzoyl)-4-[3-bromo-1,2,4-thiadiazol-5-yl]-2-isobutyl-5-(1,3-thiazol-2-yl)pyrrolidine-2-carboxylic acid;
rel-(2S,4S,5R)-1-(4-tert-butylbenzoyl)-4-(3-bromo-1,2,4-thiadiazol-5-yl)-2-isobutyl-5-(1,3-thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-thien-2-ylpyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-thien-2-ylpyrrolidine-2-carboxylic acid;
rel-(2S,4R,5R)-2-isobutyl-1-(3-methoxy-4-tert-butylbenzoyl)-4-methyl-4-(5-methyl-1,2,4-oxadiazol-3-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-oxadiazol-3-yl)-5-thien-2-ylpyrrolidine-2-carboxylic acid;
rel-(2S,4R,5R)-2-isobutyl-1-(3-methoxy-4-tert-butylbenzoyl)-4-(5-methyl-1,2,4-oxadiazol-3-yl)-5-thien-2-ylpyrrolidine-2-carboxylic acid;
rel-(2S,4S,5R)-2-isobutyl-1-(4-tert-butylbenzoyl)-4-(3-methyl-1,2,4-oxadiazol-5-yl)-5-pyridin-3-ylpyrrolidine-2-carboxylic acid, trifluoroacetate salt;
rel-(2S,4S,5R)-2-isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid;
rel-(2S,4R,5R)-2-isobutyl-1-(3-methoxy-4-tert-butylbenzoyl)-4-(5-methyl-1,2,4-oxadiazol-3-yl)-5-(1,3-thiazol-4-yl)pyrrolidine-2-carboxylic acid;
rel-(2S,4R,5R)-1-(4-tert-butylbenzoyl)-4-(1,2,4-thiadiazol-5-yl)-2-isobutyl-5-(1,3-thiazol-2-yl)pyrrolidine-2-carboxylic acid;
rel-(2S,4R,5R)-2-isobutyl-1-(3-methoxy-4-tert-butylbenzoyl)-4-(5-methyl-1,3,4-oxadiazol-2-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,3,4-oxadiazol-2-yl)-5-(1,3-thiazol-2-yl)pyrrolidine-2-carboxylic acid;
rel-(2S,4S,5R)-2-isobutyl-1-(4-tert-butylbenzoyl)-4-(5-methyl-1,3,4-oxadiazol-2-yl)-5-(1,3-thiazol-2-yl)pyrrolidine-2-carboxylic acid;
rel-(2S,4R,5R)-2-isobutyl-1-(4-tert-butylbenzoyl)-4-(5-methyl-1,3,4-oxadiazol-2-yl)-5-(1,3-thiazol-2-yl)pyrrolidine-2-carboxylic acid;
rel-(2S,4R,5R)-4-(5-ethyl-1,2,4-oxadiazol-3-yl)-2-isobutyl-1-(3-methoxy-4-tert-butylbenzoyl)-5-(1,3-thiazol-2-yl)pyrrolidine-2-carboxylic acid;
rel-(2S,4R,5R)-4-(5-cyclopropyl-1,2,4-oxadiazol-3-yl)-2-isobutyl-1-(3-methoxy-4-tert-butylbenzoyl)-5-(1,3-thiazol-2-yl)pyrrolidine-2-carboxylic acid;
rel-(2S,4S,5R)-2-isobutyl-1-(3-methoxy-4-tert-butylbenzoyl)-4-(5-methyl-1,2,4-thiadiazol-3-yl)-5-(1,3-thiazol-2-yl)pyrrolidine-2-carboxylic acid;
rel-(2S,4S,5R)-1-(4-tert-butylbenzoyl)-2-isobutyl-4-(5-methyl-1,2,4-thiadiazol-3-yl)-5-(1,3-thiazol-2-yl)pyrrolidine-2-carboxylic acid;
rel-(2S,4S,5R)-1-(3-methoxy-4-tert-butylbenzoyl)-2-methyl-4-(3-methyl-1,2,4-oxadiazol-5-yl)-5-(1,3-thiazol-2-yl)pyrrolidine-2-carboxylic acid;
rel-(2S,4R,5R)-1-(3-bromo-4-tert-butylbenzoyl)-4-(3-methyl-1,2,4-oxadiazol-5-yl)-2-isobutyl-5-pyridin-2-ylpyrrolidine-2-carboxylic acid;
rel-(2S,4R,5R)-2-isobutyl-1-(3-methoxy-4-tert-butylbenzoyl)-4-(3-methyl-isoxaxol-5-yl)-5-(1,3-thiazol-2-yl)pyrrolidine-2-carboxylic acid;
rel-(2S,4R,5R)-2-isobutyl-1-(3-methyl-4-tert-butylbenzoyl)-4-(3-methyl-1,2,4-oxadiazol-5-yl)-5-(1,3-thiazol-2-yl)pyrrolidine-2-carboxylic acid;
rel-(2S,4S,5R)-2-isobutyl-1-(3-methoxy-4-tert-butylbenzoyl)-4-(3-methyl-1,2,4-oxadiazol-5-yl)-5-(1,3-thiazol-4-yl)pyrrolidine-2-carboxylic acid;
rel-(2S,4R,5R)-2-isobutyl-1-(3-methoxy-4-tert-butylbenzoyl)-4-(3-methyl-1,2,4-oxadiazol-5-yl)-5-(1,3-thiazol-4-yl)pyrrolidine-2-carboxylic acid;
and salts, solvates and esters, and individual enantiomers thereof.

9. A pharmaceutical formulation comprising a compound of Formula (I) as claimed in claim 1 in conjunction with a pharmaceutically acceptable diluent or carrier therefor.

10. A method of treating or preventing HCV infection which comprises administering to a subject in need thereof, an effective amount of a compound as claimed in claim 1.

11. A method as claimed in claim 10 in which the compound is administered in an oral dosage form.

12. (canceled)

13. (canceled)

14. (canceled)

15. A process for the preparation of compounds of Formula (I) as claimed in claim 1, comprising
converting the moiety W of a compound of Formula (II) 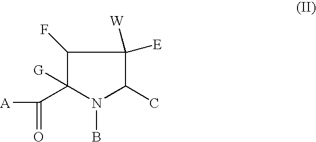
which A, B, C, E, F and G are as defined above for Formula (I); W represents W represents —CN, —CO₂H, —CO₂R⁹, —COR¹⁰, —C(O)NR⁶R⁷, or —C(O)Hal; and R⁹ represents C₁₋₆alkyl, or arylalkyl; and R¹⁰ represents C₁₋₆alkyl; into the moiety D of formula (I).
